(19) United States
(12) Patent Application Publication
(10) Pub. No.: US 2011/0144505 A1
(43) Pub. Date: Jun. 16, 2011

(54) OPTICAL DEVICE AND METHOD FOR SHAPE AND GRADIENT DETECTION AND/OR MEASUREMENT AND ASSOCIATED DEVICE

(76) Inventors: Masaki Yamamoto, Miyagi (JP); Toshihide Tsuru, Miyagi (JP)

(21) Appl. No.: 13/058,809

(22) PCT Filed: Aug. 20, 2009
(86) PCT No.: PCT/JP2009/003995

§ 371 Date: Feb. 11, 2011

(30) Foreign Application Priority Data
Aug. 28, 2008 (JP) 2008 211895

(51) Int. Cl. A61B 6/00; G01B 11/24

(52) U.S. Cl. 600/476; 356/369; 356/601

(57) ABSTRACT

Provided are: an optical device for shape and gradient detection and/or measurement which has a simple structure, is robust to external disturbance, and enables accurate measurement of the gradient angle of an object surface, including a human body; a method for optical shape and gradient detection and/or measurement; and a circularly polarized light illumination device. The optical device for shape and gradient detection and/or measurement uses the optical reflection characteristics of the surface of an object to detect and/or measure the surface shape or gradient of an observed object, and is provided with an illumination device and a polarized light image detection device. The illumination device makes the incident light, which surrounds the periphery of the object and is essentially a known perfect polarized light, fall uniformly. The polarized light image detection device detects a polarized light ellipse of the perfectly polarized light component of a light beam group, which is specularly reflected by the object surface and radiated at a particular azimuth angle. The optical device measures the gradient angle with respect to the radiated light beam of the reflection surface in a step 1 in which the orientation of the incident plane is detected from the observed azimuth angle value of the polarized light ellipse for the reflecting surface of the object which forms an incident point for each reflected and radiated light beam, and a step 2 in which the incident angle is detected from the ellipticity logic value of the polarized light ellipse. The method for optical shape and gradient detection and/or measurement is carried out using the same operation.

### TECHNICAL FIELD

The present invention relates to an optical device for shape and gradient detection and/or measurement, and to a method for extracting object information. The present invention particularly relates to an optical device for shape and gradient detection and/or measurement of an observation sample and to a method for extracting object information, the device and method being advantageous in shape-measuring microscopes, biological microscopes, shape-measuring telescopes, medical diagnostic devices, mammography devices, gradient sensors, and the like. The present invention also relates to a circular polarized light illumination device and method, and particularly relates to a circular polarized light illumination device for observation samples advantageous in shape-measuring cameras, biological microscopes, shape-measuring telescopes, devices for measuring the inner surface shape of cylinders or the like, devices for measuring aspheric shapes, mammography devices, gradient sensors, and the like.

### BACKGROUND ART

In the case that the form of a three-dimensional biological sample or another sample is to be measured using microscopy, the sample is illuminated with suitable illumination light, and an image is magnified and projected by a micro-optic system. In such a case, the projected surface is a CCD detector or another two-dimensional surface, and information related to shape in the thickness direction of the sample is usually lost. In particular, a confocal microscope has been used as a microscope for obtaining a plurality of two-dimensional cross-sectional images in the thickness direction and reconstructing a three-dimensional image using techniques capable of accentuating two-dimensionality (Hajimete Demo Dekiru Kyoushouten Kenbikyou Katsuyou Protocol [The First-Try Successful Protocol for Use in Confocal Microscopes], by Kuniaki Takata, Yodosha, December 2003, ISBN: 9784897064130 (Non-patent Document 1), International Publication WO 2004/036284 Pamphlet (Patent Document 1)). However, with this method, it is essential to sequentially capture two-dimensional images at a plurality of depths of the sample in chronological fashion, and the assumption is that the sample will not change form within the observation time period.

Also, efforts are being made to increase speed by using a method in which a special configuration is employed to scan the intensity distribution of the two-dimensional image at high speed or to perform synchronous detection using an array detector. However, this not only increases the size of the device, but also leads to significantly increased costs needed to achieve stable operation because of the more complex imaging conditions and environment.

On the other hand, interferometric methods are in use as methods capable of precisely measuring the shape of an object surface and changes in the shape. However, these methods commonly involve dividing an optical path to form observation light and reference light, and controlling the difference in the optical paths to generate interference fringes and measure the length of the optical path. Therefore, a special environment must be prepared as a countermeasure to vibrations, temperature fluctuations, and the like because the measurement values are affected by disturbances in the optical paths; and such measures cannot be applied in an ordinary environment. Also, since the shape is calculated from the distance in the optical path direction, the shape is expressed in the form of a topographical map shown by contour lines. Therefore, the surface shape, and more particularly the surface gradient, cannot be directly measured. Furthermore, moire topography and other fringe projection methods are used in actual practice to determine the shape of the surface of the human body, and measurement precision is low at about 1 mm. Sensitivity is high in hologram-based interferometric methods, but the procedures are laborious.

A measurement method has been developed for application in robotics in which the shape of an object is recognized using the fact that polarization is generated at the polarizing angle of reflection of the surface of a transparent object, i.e., the reflected light has a greater s-polarized component than a p-polarized component when the object shape is illuminated with unpolarized light (Non-patent Document 2: Recovery of Surface Orientation From Diffuse Polarization, G. Atkinson and E. R. Hancock, IEEE Transaction of Image Processing, Vol. 15, No. 6, June 2006). Polarized light measurement for shape recognition belongs to the field of polarimetry for measuring partially polarized light, which is carried out in robotics applications under that assumption that natural light (unpolarized light) is used for illumination with consideration given to practical utility. Circularly polarized light was used in the initial development carried out by Koshikawa (Non-patent Document 3: A Polarimetric Approach to Shape Understanding of Glossy Objects, K. Koshikawa, Proc. Int. Joint Conf. Art. Intell., pp. 493-495 (1979); Patent Document 2: Japanese Patent Publication (Kokoku) No. 61-17281 “Method for Detecting Direction of Glossy Surface”). However, the measurement method is a polarimetric method that involves partially polarized light, and although measurement sensitivity can fundamentally be obtained with a transparent body, sensitivity cannot be obtained with a metal surface. Therefore, subsequent development was limited to simple measurements for measuring the degree of polarization under illumination with unpolarized light.

Although these methods are capable of reproducing the shape of a transparent body, the precision of the measured angle is on the level of several degrees. With metal materials, the difference in reflection intensity is low because the reflectance of the p-polarized component at a polarization angle merely assumes a minimum value without reaching zero, and application is fundamentally impossible.

Polarized light refers to light in which the electric and magnetic fields oscillate with polarization in a specific direction. Classified by the manner in which polarization changes over time, polarized light is generally elliptically polarized light, but there are also linearly polarized light and circularly polarized light. Light is an electromagnetic wave, and the electromagnetic field is a transverse wave that oscillates perpendicular the travelling direction. In linearly polarized light, the direction of oscillation of the electric field (and magnetic field) is constant, and the plane of oscillation of linearly polarized light refers to the direction of the electric field. Circular polarized light describes a circle in accompaniment with the propagation of oscillations of the electric field (and magnetic field), and is right circularly polarized light or left circularly polarized light, depending on the direction of rotation. Elliptically polarized light is the most common polarization state expressed by the primary coupling of linearly polarized light and circularly polarized light, and the oscillation of the electric field (and magnetic field) describes an ellipse in relation to time. Elliptically polarized light is right elliptically polarized light or left elliptically polarized light. Light (electromagnetic waves) perpendicular to the plane on which the electric field component is incident is referred to as an s-wave (σ-light, which is perpendicular to the incident plane), and light (electromagnetic wave) parallel to the plane on which the electric field component is incident is referred to as a p-wave (π-light, which is parallel to the incident plane). Light that is clockwise facing the travelling direction is referred to as left circularly polarized light (leftward rotation as viewed from the perspective of the receiver of the light), and light that is counterclockwise facing the travelling direction is referred to as right circularly polarized light (rightward rotation as viewed from the perspective of the receiver of the light). In particular, polarized light in which there is only one type of change of polarization over time is referred to as perfectly polarized light. The sum of perfectly polarized light and unpolarized light devoid of polarization is referred to as partially polarized light.

Another method for measuring polarized light capable of high measurement precision in contrast to polarimetry is ellipsometry. Polarimetry measures partially polarized light containing an unpolarized component that accompanies light scattering and the like, whereas ellipsometry has high measurement precision because the shape of the polarized light ellipse, which indicates the polarization state of the perfectly polarized light, is used as the target of measurement in order to handle reflection from a surface that is sufficiently smooth to not produce scattering. Methods for measuring and analyzing polarized light can be found in “Henko Sokutei to Henko Kaisekiho” (Measurement and Analysis of Polarized Light) by Masaki Yamamoto; “Hikari Kogaku Handbook” (Handbook of Optical Engineering) by Teruji Kose, et al., Asakura Shoten, 1986, pp. 411-427 (Non-patent Document 4); and “Jiku Taisho Henko Bimu” (Axisymmetric Polarized Light Beams) by Yuichi Kozawa and Shunichi Sato, Kogaku, Vol. 35, No. 12 (2006), pp. 9-18 (Non-patent Document 5).

Various interferometric methods, moire topography, and other fringe projection methods, as well as confocal microscopy are widely used in actual practice as methods for optically measuring the three-dimensional-shape of an object. These optical measurement methods essentially involve measuring distances to obtain a shape in the form of a topographical map shown by contour lines. Measurement methods that do not depend on distance measurement are under development in recognition research in the field of robotics in relation to three-dimensional shapes. Such research uses “polarization” of scattered light from an object surface under illumination with unpolarized light, and there has been success in reconstructing shapes (Non-patent Document 6). Reference can also be found in a patent specification (Patent Document 3: Japanese Laid-open Patent Application No. 11-211433). The measurement principles for these shape recognition applications is polarimetry, that is, a gradient measurement method that make use of the fact that dependence on the gradient angle for the degree of polarization, which indicates “polarization” of scattering from the surface of snow, approaches a maximum value of 1 at the polarization angle of water. Although applications are currently limited to shape recognition because the measurement accuracy of the degree of polarization is only several percent, there are indications that the “gradient” can be directly obtained by using polarized light to reconstruct the shape of an object in real time.

Shape recognition research in relation to a glossy transparent scattering body was conceived by Koshikawa for robotics applications in 1979 (Non-patent Document 3 and Patent Document 2: Japanese Patent Publication (Kokoku) No. 61-17281), and although illumination with circularly polarized light was used in experimentation for verifying basic principles, partially polarized light was measured using scattering samples. Therefore, illumination with unpolarized light was used exclusively thereafter, and development did not progress in the direction of precisely measuring shapes. However, development of a polarized camera (Non-patent Document 7) has made progress in the detection of polarized light images required for real time measurement in robotics applications, and applications for extracting various object shapes have also been developed.

On the other hand, ellipsometry is known as a method for making precision measurements using polarized light. Ellipsometry precisely measures the optical properties of a sample or the thickness of a thin film on the basis of changes in the polarization state when linearly polarized light is used as a probe and is directed diagonally onto and reflected from a flat sample. In the field of ellipsometry, the principal angle of incidence method (Non-patent Document 4) is a known method used for precisely measuring the dependency of the polarized light reflection characteristics on the angle of incidence, and is a technique for standard purposes such as measuring the optical characteristics of a sample. The measurement objects are limited to flat samples.

The inventors are well-versed in optical characteristics related to the incident angle dependency on the polarization state after reflection because of inventions (Patent Document 4: Japanese Patent Publication (Kokoku) No. 52-46825; Patent Document 5: Japanese Patent Publication (Kokoku) No. 60-41732; and Patent Document 6: Japanese Patent Publication (Kokoku) No. 2-16458) and applications of polarized light analysis based on the principal angle of incidence method.

### PRIOR ART DOCUMENTS

### Patent Documents

Patent Document 1: International Publication No. 2004/036284 Pamphlet (WO2004/036284, A)
Patent Document 2: Japanese Patent Publication (Kokoku) No. 61-17281
Patent Document 3: Japanese Laid-open Patent Application (Kokoku) No. 11-211433
Patent Document 4: Japanese Patent Publication (Kokoku) No. 52-46825
Patent Document 5: Japanese Patent Publication (Kokoku) No. 60-41732
Patent Document 6: Japanese Patent Publication (Kokoku) No. 2-16458
Patent Document 7: Japanese Patent Application (Tokugan) No. 2008-211895

### Non-Patent Documents

Non-patent Document 1: (Hajimete Demo Dekiru Kyoushouten Kenbikyou Katsuyou Protocol [The First-Try Successful Protocol for Use in Confocal Microscopes], by Kuniaki Takata, Yodosha, December 2003, ISBN: 9784897064130
Non-patent Document 2: G. Atkinson and E. R. Hancock, “Recovery of Surface Orientation From Diffuse Polarization”, IEEE Transaction of Image Processing, Vol. 15, No. 6, pp. 1653-1664, June (2006)
Non-patent Document 3: K. Koshikawa, “A Polarimetric Approach to Shape Understanding of Glossy Objects”, Proc. Int. Joint Conf. Art. Intell., pp. 493-495 (1979)
Non-patent Document 4: M. Yamamoto “Henko Sokutei to Henko Kaisekiho” (Measurement and Analysis of Polarized Light); T. Kose, et al., “Hikari Kogaku Handbook” (Handbook of Optical Engineering) Asakura Shoten, 1986, pp. 411-427
Non-patent Document 5: Y. Kozawa and S. Sato, “Jiku Taisho Henko Bimu” (Axisymmetric Polarized Light Beams), Kogaku, Vol. 35, No. 12 (2006), pp. 9-18
Non-patent Document 6: S. Kawakami “Sekisogata Photonic Kessho no Sangyoteki Shoyo” (Industrial Applications for Layered Photonic Crystals) Ouyou Butsuri (Applied Physics), 77, 508-514 (2008)
Non-patent Document 7: “Seihansya ni yoru Buttai Hyomen no Keisha Ellipsometry—Seimitsu Jitsu Jikan Keijo Keisoku e no Kihon Gainen” (Gradient Ellipsometry of Object Surfaces by Specular Reflection—Basic Concepts for Precise Real Time Shape Measurement) Kogaku, Vol. 38, No. 4 (2009) pp. 204-212
Non-patent Document 8: K. Kinoshita and M. Yamamoto, “Principal Angle-of-Incidence Ellipsometry”, Surf. Sci. 56, 64-75 (1976)

### DISCLOSURE OF THE INVENTION

### Problems to be Solved by the Invention

Conventional interferometry and other methods for measuring the three-dimensional shape of an object are geometrical triangulation methods in which the change in the length, i.e., the change in distance of the optical path is precisely measured. For example, the L determination error is also a gradient error because also computed is the observed value in a position set at a distance equal to a predetermined distance L on an observed object in order to calculate the gradient. Also, the observed optical path is readily affected by external disturbances while light is propagating.

An object of the present invention is to provide an optical device for shape and gradient detection and/or measurement that can withstand external disturbances in a simple manner and precisely detect and/or measure the gradient angle of the surface of an object including the human body; and to provide a method for detecting and/or measuring optical shapes and gradients. Another object is to provide a circular-polarized-light illumination device and method used in gradient/shape measurement methods for measuring the shape of the objects and that make it possible to assure measurement precision.

### Means for Solving the Problems

The present inventors, as a result of thoroughgoing research, perfected the present invention having succeeded in finding that in a fundamental principle of ellipsometry, in which the shape of a polarized light ellipse of a perfectly polarized component of reflected light varies because the change in amplitude and phase differs due to the p-component and the s-component of the electric vector of the light when perfectly polarized light having an already-known polarized light ellipse shape is reflected at a material surface, it is possible to use the fact that (1) “the reference for changes in the azimuth angle of the polarized light ellipse is the orientation of the incident plane,” and (2) “the change in ellipticity of the polarized light ellipse is a monotone function with respect to the incident angle” to measure the polarized light ellipse of reflected light, whereby the surface of a substance as the sample, and the vicinal faces, i.e., the gradients of the tangent plane reflecting the incident light can be theoretically determined by a first step for acquiring the orientation of the incident plane from the value of the observed azimuth angle of the polarized light ellipse, and a second step for theoretically calculating the angle of incidence from the measured value of ellipticity of the polarized light ellipse, and three-dimensional shapes can be reproduced by smoothly connecting gradients of the vicinal faces thus determined because the surface of the observed substance can be made to be continuous within the observation field of view.

The present invention provides the following aspects.

(1) An optical device for shape and gradient detection and/or measurement that detects and/or measures a shape and gradient of a surface of an observed object using the reflectance optical characteristics of the surface of the object, the optical device for shape and gradient detection and/or measurement characterized in comprising an illumination device for causing light surrounding a periphery of the object to be uniformly incident, the light being in a polarized state which includes a substantially already-known perfectly polarized state; and a polarized light image detection device for detecting a polarized light ellipse of a polarized light component, which includes a perfectly polarized component of a group of light beams specularly reflected by the object surface and emitted at a specific azimuth angle, wherein a gradient angle with respect to the radiated light beam of the reflection surface is measured by a step 1 in which the orientation of the incident plane is detected from the observed azimuth angle of the polarized light ellipse for the refection surface of the object that forms an incident point for each reflected and radiated light beam, and a step 2 in which the incident angle is detected from the ellipticity value of the polarized light ellipse, which includes the theoretical ellipticity value of the polarized light ellipse.

(2) The optical device for shape and gradient detection and/or measurement optical device for shape and gradient detection and/or measurement according to (1) described above, characterized in that the illumination device for causing light surrounding the periphery of the object to be uniformly incident, the light being in a polarized state which includes a substantially already-known perfectly polarized state, illuminates circular polarized light, which includes the perfectly circular polarized light.

(3) The optical device for shape and gradient detection and/or measurement according to (1) or (2) described above, characterized in that step 1, in which the orientation of the incident plane is detected from the observed azimuth angle of the polarized light ellipse, (1) detects the orientation of the incident plane from the observed azimuth angle of the polarized light ellipse, which includes the observed azimuth angle theoretical value of the polarized light ellipse, or (2) causes the right circularly polarized light and left circularly polarized light to be incident in a switching fashion in an illumination device for causing light surrounding the periphery of the object to be uniformly incident, the light being in a polarized state which includes a substantially already-known perfectly polarized state, whereby the incident plane orientation is identified by making use of the fact that the observed azimuth angle of the reflected polarized light ellipse, which includes the theoretical value of the observed azimuth angle of the reflection polarized light ellipse, is switched in symmetrical fashion to the incident plane regardless of the reflection optical characteristics of the surface of the object.

(4) The optical device for shape and gradient detection and/or measurement according to any of (1) to (3) described above, characterized in that the illumination device for causing light surrounding the periphery of the object to be uniformly incident, the light being in a polarized state which includes a substantially already-known perfectly polarized state, includes spatially specified incident light beams as a reference origin of measurement and is capable of specifying the optical characteristics of the reflection surface from the observed value of the polarized light ellipse at a reflection point specified by the polarized light image detection device.

(5) The optical device for shape and gradient detection and/or measurement according to any of (1) to (4) described above, characterized in that the polarized light image detection device for detecting the polarized light ellipse of a group of light beams reflected by the object surface and emitted at a specific azimuth angle comprises a mechanism capable of extracting an azimuth angle range of the group of light beams having essentially the same polarized light ellipse.

(6) The optical device for shape and gradient detection and/or measurement according to any of (1) to (5) described above, characterized in that the polarized light image detection device for detecting polarized light ellipses of a group of light beams reflected at the object surface and emitted at a specific azimuth angle has a structure for spatially dividing the reflected light into a plurality of at least three or more groups, assigning a plurality of detectors that can detect specific and mutually different polarized light ellipses, and simultaneously detecting in parallel the polarized light ellipses.

(7) The optical device for shape and gradient detection and/or measurement according to any of (1) to (6), characterized in comprising a crossed linearly polarized light image detection unit for causing reflected light to be divided by a polarized light beam splitter into a p-component that travels directly forward and a reflected s-polarized light component, causing each of the components to be formed into an image on a two-dimensional detector by an imaging lens, and for drawing out an object image as a crossed polarized light image output.

(8) The optical device for shape and gradient detection and/or measurement according to any of (1) to (7), characterized in that the polarized light image detection device for detecting polarized light ellipses of a group of light beams reflected at the object surface and emitted at a specific azimuth angle has a mechanism for specifying a light beam position on the object surface by obtaining a reduced projection image of the object.

(9) The optical device for shape and gradient detection and/or measurement according to any of (1) to (7), characterized in that the polarized light image detection device for detecting polarized light ellipses of a group of light beams reflected at the object surface and emitted at a specific azimuth angle has a mechanism for specifying a light beam position on the object surface by obtaining a magnified projection image of the object.

(10) The optical device for shape and gradient detection and/or measurement according to any of (1) to (7), characterized in that the polarized light image detection device for detecting polarized light ellipses of a group of light beams reflected at the object surface and emitted at a specific azimuth angle has a mechanism for specifying a light beam position on the object surface by providing a collimator.

(11) The optical device for shape and gradient detection and/or measurement according to any of (1) to (7), characterized in that the polarized light image detection device for detecting polarized light ellipses of a group of light beams reflected at the object surface and emitted at a specific azimuth angle has a mechanism for specifying a light beam position on the object surface by arranging the device essentially at infinite distance.

(12) The optical device for shape and gradient detection and/or measurement according to any of (1) to (7), characterized in that the polarized light image detection device for detecting polarized light ellipses of a group of light beams reflected at the object surface and emitted at a specific azimuth angle has a mechanism for specifying a light beam position on the object surface by providing a pinhole.

(13) The optical device for shape and gradient detection and/or measurement according to any of (1) to (12), characterized in being a medical diagnostic device including mammography for detecting and identifying a specific change in a surface gradient angle caused by a variety of pathological abnormalities including malignant tumors, an object of detection and/or measurement being a human body or a portion of a human body including a breast.

(14) The optical device for shape and gradient detection and/or measurement according to any of (1) to (13), characterized in that dynamic characteristics are extracted by imparting deformation caused by a predetermined stress by a dynamic process including a change in orientation of the observed object, which includes a patient, and detecting and/or measuring changes in the gradient angle before and after deformation.

(15) The optical device for shape and gradient detection and/or measurement according to any of (1) to (14), characterized in that a change in the optical characteristics of a reflection surface is detected and/or measured using the illumination light as white light and the surface of an observed object, including skin, as a substantially reflective surface, taking into account that the depth of penetration from such a surface changes with the wavelength.

(16) A method for optical shape and gradient detection and/or measurement to detect and/or measure a shape and a gradient of a surface of an observed object using the reflectance optical characteristics of the surface of the object, the method for optical shape and gradient detection and/or measurement characterized in comprising: using an illumination device to cause light surrounding a periphery of the object to be uniformly incident, the light being in a polarized state which includes a substantially already-known perfectly polarized state; using a polarized light image detection device to detect a polarized light ellipse of a polarized light component, which includes a perfectly polarized component of a group of light beams specularly reflected by the object surface and emitted at a specific azimuth angle; measuring a gradient angle with respect to the radiated light beam of the reflection surface by detecting the orientation of the incident plane from the observed azimuth angle of the polarized light ellipse for the refection surface of the object that forms an incident point for each of the reflected and radiated light beams, and detecting the incident angle from the ellipticity value of the polarized light ellipse, which includes the theoretical ellipticity value of the polarized light ellipse; the NA of the optical system of the detection device being set to the maximum value or to a function value in relation to the measurement precision of the polarization state; and extracting object information using the fact that the measured gradient angle smoothly varies on the object surface.

(17) The method for optical shape and gradient detection and/or measurement according to (16), characterized in that a specific change in the surface gradient angle caused by a variety of pathological abnormalities, including malignant tumors, is detected and identified, the object of detection and/or measurement being a human body or a portion of a human body including a breast.

(18) The method for optical shape and gradient detection and/or measurement according to (16) or (17), characterized in that a predetermined deformation is imparted by a process that includes changing an orientation of the observed body, which includes a patient, and detecting and/or measuring a change in the gradient angle before and after deformation.

(19) The method for optical shape and gradient detection and/or measurement according to any (16) to (18), characterized in that a change in the optical characteristics of a reflection surface is detected and/or measured using the illumination light as white light and the surface of an observed object, including skin, as a substantially reflective surface, taking into account that the depth of penetration from such a surface changes with the wavelength.

(20) A method for detecting and/or measuring a shape and gradient, characterized in comprising an optical device for shape and gradient detection and/or measurement, used to detect and/or measure a shape and gradient of a surface of an observed object using reflectance optical characteristics of the surface of the object, having: an illumination device for causing light surrounding a periphery of the object to be uniformly incident, the light being in a polarized state which includes a substantially already-known perfectly polarized state; and a polarized light image detection device for detecting a polarized light ellipse of a polarized light component, which includes a perfectly polarized component of a group of light beams specularly reflected by the object surface and emitted at a specific azimuth angle; measuring the gradient angle in relation to light beams radiated from the reflection surface by detecting: the azimuth angle of the incident plane, i.e., the azimuth angle of the normal of the tangent plane, from the azimuth angle of the polarized light ellipse for the reflection surface, i.e., the vicinal face, of the object that forms an incident point for each of the reflected and radiated light beams; and the reflection angle, i.e., the incident angle from the ellipticity value of the polarized light ellipse; and carrying out an integration operation for smoothly connecting the vicinal faces that form the tangent plane.

(21) The method for optical shape and gradient detection and/or measurement according to (20), characterized in comprising directly measuring a reflection angle formed with an axis that is an observation direction, and a polarization angle of a projection component on the plane perpendicular to the axis that is the observation direction, for the normal of the tangent plane at the reflection point of the observed object surface, using incident angle dependency of a variation in the polarized light ellipse formed with a single reflection.

(22) The method for optical shape and gradient detection and/or measurement according to (20) or (21), characterized in comprising establishing a partial derivative coefficient at the coordinates of the axis component that is the observation direction as the gradient of the tangent plane at the reflection point on the surface of the observed object.

(23) The method for optical shape and gradient detection and/or measurement according to any of (20) to (22), characterized in comprising measuring a slope of the normal of the tangent plane at the reflection point on the surface of the observed object; calculating the partial derivative coefficient of the shape and gradient at the reflection point on the object, measuring temporal changes and/or spatial changes in the partial derivative coefficient; and extracting characteristics of the shape and/or characteristics of the gradient by directly using measured values that have been obtained.

(24) The method for optical shape and gradient detection and/or measurement according to any of (20) to (23), characterized in comprising measuring the gradient of the tangent plane and the shape of the observed object by ellipsometry using the complex amplitude reflectivity ratio calculated using an optical model that expresses optical properties of the observed sample, and the values Ψ, Δ obtained from the ellipticity angle of the reflected polarized light ellipse and from the azimuth angle of the major axis.

As a result of measurement errors and carrying out an analysis of the cause of such errors in the process of researching and developing a measurement method for a novel invention in which the optical characteristics of a sample are known and the geometric orientation of the sample surface is unknown, that is to say, an invention of a gradient and shape measurement method for measuring the shape and gradient of an object, in which circularly polarized light is made incident on the gradient surface constituting the object surface, and the gradient plane and a three-dimensional gradient angle of the gradient plane are formed using the polarized light characteristics of reflected light beams reflected in an specified observation direction, it was found that measurement precision can be assured by adopting a novel configuration for the specification of a circularly polarized light illumination device. In other words, the circularly polarized light device of the present invention is capable of achieving ellipsometric precision of <1% in precision shape measurement by three-dimensional gradient ellipsometry proposed by the inventors. This precision considerably improves on conventional polarized light measurement by several percent.

The inventors found that precision shape measurement by three-dimensional gradient ellipsometry can be applied to shape and gradient measurement of the surface of an object including the inner surface, to which conventional optical shape measurement cannot be applied. In particular, it was found that precision optical measurement can be applied to cases in which optical methods cannot be applied for the inner surface of a cylindrical object, the inner surface of a cylindrical object having one sealed off, and the like.

In the precision shape and gradient measurement by three-dimensional gradient ellipsometry proposed by the present inventors, imperfections are eliminated in circular polarized illumination, which is the source of measurement errors, and it possible to obtain a configuration for achieving a measurement precision of 1% to 0.1% in the promising field of ellipsometry. It is also possible to carry out precision optical measurements of gradients and shapes of an object surface, including the inner surface.

Therefore, the present invention furthermore provides the following aspects.

(25) A circularly polarized light illumination device used in shape and gradient measurement methods for measuring the shape and gradient of an object, the circularly polarized light illumination device characterized in that: the shape and gradient of the object are measured by making circularly polarized light incident on a gradient plane constituting the object surface, including the inner surface, and using the polarized light characteristics of reflected light beams specularly reflected in a specified observation direction, to form the gradient plane and a three-dimensional gradient angle of the gradient plane, wherein the circularly polarized light illumination device comprises a light source device; and the light source device is a light source device having illumination sections with circular shapes, rectangular shapes, or a combination thereof in polyhedral shapes that include a flat surface or a curved surface directly facing the object, wherein the sections include concave surfaces surrounding an outer surface of the object or convex surfaces facing an inner surface of an object; circularly polarized light including essentially perfect circularly polarized light can be irradiated toward the object via the sections; and a group of circularly polarized light beams made incident on the object surface is made to include all incident light beam components that can be specularly reflected in the observation direction in accordance with the law of reflection.

(26) The circularly polarized light illumination device according to (25), characterized in that the light source device having the illumination sections includes, in the stated order, a light source, optical elements for directing light to the sections, and circular polarizers; and is provided with a function enabling emitting of circularly polarized light, including perfect circularly polarized light having a predetermined degree of polarization, from the sections as incident angle light beam flux in a predetermined angle range.

(27) The circularly polarized light illumination device according to (25) or (26), characterized in that the light source device having the illumination sections is capable of illuminating the object with circularly polarized light beam flux in which the degree of polarization is essentially 99% or higher.

(28) The circularly polarized light illumination device according to any of (25) to (27), characterized in that illumination sections of the light source device form polyhedral sections having any regular polygonal shape or a combination thereof inscribed in a circle.

(29) The circularly polarized light illumination device according to any of (25) to (28), characterized in that the light source device has optical fiber elements arranged at predetermined angles and causes light to be perpendicularly incident on the illumination sections.

(30) The circularly polarized light illumination device according to any of (25) to (29), characterized in that the light source device having the illumination sections includes at least a substantially planar light source in which point light sources are arrayed, and/or a surface-emitting light source, and circular polarizers in the stated order.

(31) The circularly polarized light illumination device according to any of (25) to (30), characterized in that the light source device includes a light source mechanism for generating light flux that diverges from at least a single point and a rotating ellipsoidal reflection mirror; the divergence point and the position of the object are arranged in alignment with the focal point of the rotating ellipsoidal reflection mirror; and light is made to be perpendicularly incident on the illumination sections by causing the illumination light beams to converge on the object by reflection.

(32) The circularly polarized light illumination device according to any of (25) to (30), characterized in that the light source device includes a light source mechanism for generating at least parallel illumination light flux and a rotating parabolic mirror; the position of the object is arranged in alignment with the focal point of the rotating parabolic mirror; and light is made to be perpendicularly incident on the illumination sections by causing the illumination light beams to converge on the object by reflection.

(33) The circularly polarized light illumination device according to any of (25) to (32), characterized in comprising an illumination angle origin reference within the illumination sections of the light source device.

(34) The circularly polarized light illumination device according to any of (25) to (33), characterized in comprising a function for temporally or spatially selecting a circularly polarized light state of the illumination light flux using right circularly polarized light or left circularly polarized light.

(35) A circularly polarized light illumination method used in shape and gradient measurement methods for measuring the shape and gradient of an object in which circularly polarized light is made to be incident on a gradient plane constituting the object surface, including the inner surface, and the polarized light characteristics of reflected light beams specularly reflected in a specified observation direction are used to form the gradient plane and a three-dimensional gradient angle of the gradient plane, the circularly polarized light illumination method characterized in comprising: using a light source device having illumination sections with circular shapes, rectangular shapes, or a combination thereof in polyhedral shapes that include a flat surface or a curved surface directly facing the object, wherein the sections include concave surfaces surrounding the outer surface of the object or convex surfaces facing the inner surface of an object; irradiating circularly polarized light including essentially perfect circularly polarized light toward the object via the sections; and causing a group of circularly polarized light beams made incident on the object surface to include all incident light beam components that can be specularly reflected in the observation direction in accordance with the law of reflection.

### Effect of the Invention

In accordance with the present invention, a two-dimensional polarized light image of an object recorded by a microscope, a telescope, a projection device, or another image formation device can be analyzed, and the gradient angle (0° to 90°) of the surface constituting the object can be detected and/or measured with a precision of 0.01° to 0.001°. It is possible to obtain a device for detecting and/or measuring three-dimensional shapes and gradients, in which the three-dimensional shape of an object can be reconstructed using the measured gradient angles by making use of the fact that the object surfaces are smoothly connected together. Also, the device for this purpose does not require a complex mechanism, and is a simple device for detecting and/or measuring three-dimensional shapes and gradients.

In the present invention, the gradient angle of a reflection surface is observed using the reflection of polarized light. Change in the polarization state due to reflection is a phenomenon occurs only once in a measurement section. Excluding this reflection phenomenon, change does not occur in the polarization state during the propagation of light. Since the incident polarized light and the output polarized light after reflection both pass through air, liquid, or another isotropic uniform medium, change does not occur in the polarization state during propagation of light. Therefore, the most important feature is the point that [polarized light] is not affected by the observation environment and the observation distance.

In the present invention, the gradient of an object can be read directly. Various applications are possible by simple image processing because local variations in the gradient can also be precisely observed without contact. In particular, since the environment is irrelevant, a novel device for detection and measurement can be provided and used in dynamic measurement of nanosized samples under a microscope, medical diagnoses in humans such as extraction of local depressions in a mammary surface due to a malignant tumor, measurement applications by satellite image, and in a wide range of other applications.

In accordance with the present invention, it is possible to obtain a circular polarized light illumination device that is capable of advantageous use in three-dimensional shape and gradient measurement devices that can analyze the two-dimensional polarized light image of a sample recorded by a polarized light camera, and precisely measure the three-dimensional gradient and shape of a surface, including the inner surface of a sample. In particular, it possible to obtain a configuration for achieving a measurement precision of 1% to 0.1% in the promising field of ellipsometry.

Other objects, characteristics, advantages, and aspects of the present invention will be apparent to one skilled in the art from the description given below. However, it should be understood that the following description and the description of the present specification, which includes specific examples and the like, merely show preferred modes of the present invention and are given only by way of explanation. It will be clearly apparent to one skilled in the art from the information given in the following description and other portions of the present specification that various changes and/or improvements (or modifications) are possible within the intention and scope of the present invention as disclosed in the present specification. All patent references and other references cited in the present specification are cited for descriptive purposes, and the contents of the references shall be construed as being included in the disclosure of the present specification as part of the present specification.

### BRIEF DESCRIPTION OF THE DRAWINGS

FIG. 1 shows the state in which light rays (bold lines) incident on a spherical sample are reflected at each reflection point and reflected in the observation orientation, wherein the observation orientation is the z-axis direction, the reflected light rays are parallel to the z-axis according to the law of reflection in the case of reflection in the xy plane, and the sample is assumed to be transparent;

FIG. 2 schematically shows the observation plane (a circle) of a spherical sample and the polarization state at the point of observation inside a circle, which is the plane of observation of the spherical sample, wherein the polarization state is indicated by the ellipses in the drawing, the orientation of the major axis of the ellipses is orthogonal to the incident plane, light passes through a state of linearly polarized light having ellipticity ∈=0 in the vicinity of the polarization angle φ=56° for a medium having a refractive index n=1.5, which is the angle formed by the normal line to the tangent plane with the z-axis as the observation orientation, the inner region (the region in which the angle of incidence is less than the polarization angle of a specific medium) of the linearly polarized light is therefore indicated by circular shading, the shaded region is left polarized light, the peripheral area of the sample observation plane outside of the shaded region is right polarized light, and the sample is transparent, corresponding to the case in which the sample is a transparent cell or the like;

FIG. 3 schematically shows the observation plane of a spherical sample corresponding to FIG. 2 for a case in which there is absorption in the sample, and the polarization state at the point of observation inside a circle, which is the plane of observation of the spherical sample, wherein the polarization state is indicated by the ellipses in the drawing, and the azimuth angle of the ellipses is believed to be rotated a predetermined angle, corresponding to the case in which the sample is aluminum or another absorbing body or the like;

FIG. 4 shows the results of calculating the relationship between the angle of incidence and tan Ψ cos Δ and tan Ψ sin Δ of the polarization state for the case in which the sample is glass having a refractive index of n=1.5 in air, which corresponds to the case in which the sample is a transparent cell or the like;

FIG. 5 shows the results of calculating the relationship between the angle of incidence and the intensity reflectance of the p-s polarized light components for the case in which the sample is glass having a refractive index of n=1.5 in air, which corresponds to the case in which the sample is a transparent cell or the like;

FIG. 6 shows the results of calculating the relationship between the incident angle and the complex amplitude reflectance ratio Rp/Rs for the case in which the sample is an absorbing body, and shows a complex plane plot at a wavelength of 405 nm (the wavelength of a blue light-emitting diode) obtained on the assumption that an oxidized aluminum surface is used as the sample, wherein the complex refractive index is 0.6 to 5.04i due to absorption, corresponding to the case in which the sample is a metal or the like;

FIG. 7 shows the results of calculating the relationship between the incident angle and Ψ and Δ of the complex amplitude reflectance ratio Rp/Rs for the case in which the sample is an absorbing body, wherein the results are obtained for a wavelength of 405 nm (the wavelength of a blue light-emitting diode) on the assumption that an oxidized aluminum surface is used as the sample, and the complex refractive index is 0.6 to 5.04i due to absorption, corresponding to the case in which the sample is a metal or the like;

FIG. 8 shows the results of calculating the relationship between the incident angle and the intensity reflectance of the p-s polarized light components for the case in which the sample is aluminum (having an oxidized aluminum surface) in air, wherein the complex refractive index is 0.6 to 5.04i due to absorption, corresponding to the case in which the sample is a metal or the like, at a wavelength of 405 nm (the wavelength of a blue light-emitting diode);

FIG. 9 shows the configuration of a shape-measuring telescope, which is one of the shape-measuring optical devices of the present invention, and shows the device in the most simple and basic configuration;

FIG. 10 shows the configuration of a shape-measuring microscope, which is one of the shape-measuring optical devices of the present invention, and shows the device in the most simple and basic configuration;

FIG. 11 shows the configuration of a shape-measuring optical device of the present invention, and shows the device in a most simple and basic configuration;

FIG. 12 shows a configuration example of a shape-measuring optical device of the present invention;

FIG. 13 shows another configuration example of the shape-measuring optical device of the present invention;

FIG. 14 shows a configuration example of a mammography device, which is a shape-measuring optical device of the present invention;

FIG. 15 shows another configuration example of the mammography device, which is a shape-measuring optical device of the present invention;

FIG. 16 shows a configuration of an orthogonal unit used in the present invention;

FIG. 17 schematically shows a configuration example of the shape-measuring optical device of the present invention;

FIG. 18 schematically shows yet another configuration example of the shape-measuring optical device of the present invention;

FIG. 19 is diagram for describing the case in which concepts of ellipsometry defined in a two-dimensional plane are expanded to specular reflection on a surface including the inner surface of a three-dimensional object, wherein a “bright” specularly reflected normal zero-order light component that satisfies the law of reflection is present at an arbitrary reflection point in the surface viewed from the z direction when the object surface is uniformly illuminated by circularly polarized light and specularly reflected light is observed from the z direction, the incident plane is defined as the plane that includes the incident light beam and the normal line of the refection surface, the normal vector perpendicular to the reflection surface is invariably included in the incident plane, the reflection angle (=incident angle) is equal to the angle formed by the normal vector with the z-axis, and the normal vector is determined when the azimuth angle of the incident plane and the incident angle can be determined for any light beam travelling in the z direction;

FIG. 20 shows a reflected polarized light ellipse observed from the z direction under illumination with circularly polarized light for the case (a) in which the reflection is from a dielectric sample and the case (b) in which the reflection is from a metal sample;

FIG. 21 shows a conversion table of the incident angle cosine and the observed ellipticity angle with right circular polarized light incidence;

FIG. 22 shows a device used in experimentation of the gradient and slope measurement method for measuring the three-dimensional gradient angle of the gradient plane and the shape of an object that forms the gradient plane by causing circular polarized light to be incident on the gradient plane constituting the object surface and using the polarized light characteristics of the reflected light beams specularly reflected in a specified observation direction;

FIG. 23 shows the results of observing a prismoid and a hemisphere on the left and right using the device of FIG. 19, wherein a) is the observed ellipticity angle, b) is the observed azimuth angle, and c) is a photograph of the samples;

FIG. 24 shows the variation in intensity of transmitted light as a solid line for the case in which a polarizer and an analyzer are arranged on straight line, the transmission axis of the polarizer is fixed at an azimuth angle of 0°, and the orientation of the transmission axis of the analyzer is θ, wherein the variation in intensity is indicated by the broken line in accordance with the logarithmic scale on the right vertical axis;

FIG. 25 shows Malus' law for polarizers of various extinction rates in terms of changes in the azimuth angle near the extinction position of an observed intensity I;

FIG. 26 fundamentally shows the incident angle dependency of the phase angle of a retarder that uses birefringence, and for such a case, shows that the allowable angle range is limited with dependency on the required precision;

FIG. 27 shows an example in which the relationship between the incident angle and the phase angle of the retarder for average refractive indexes of 1.5, 1.4, and 1.0;

FIG. 28 shows that the incident angle or the output angle of light beams in relation to the polarizer must be kept within a predetermined allowed angle range in order to generate perfectly circular polarized light with predetermined precision, and that this requirement can be satisfied when a regular polygon inscribed within a circle indicating the allowed angle is used as an element;

FIG. 29 shows an example of illumination partitions compactly configured by laminating a circular polarizer to a surface-luminous light source;

FIG. 30 shows an example of the case of a configuration having an illumination angle origin reference inside the illumination partition of a light source device;

FIG. 31 shows configuration examples of the illumination region formed by the illumination partition and regular polygons facing the measurement object in a light source device;

FIG. 32 shows an example of a configuration of a mode in the light source device in which a fiber light source has been combined with a configuration in which the illumination region is formed by an illumination partition and a regular octahedron facing the measurement object;

FIG. 33 shows a specific example of the circular polarized light illumination device of the present invention;

FIG. 34 shows another specific example of the circular polarized light illumination device of the present invention;

FIG. 35 shows specific example of inner surface shape observation in accordance with the present invention;

FIG. 36 shows another specific example of inner surface shape observation in accordance with the present invention;

FIG. 37 shows a specific example in accordance with the present invention for observing the shape of an inner surface in which one end has been sealed off;

FIG. 38 shows another specific example in accordance with the present invention for observing the shape of an inner surface in which one end has been sealed off;

FIG. 39 shows another specific example in accordance with the present invention for observing the shape of an inner surface for an example in which the shape of the inner surface of a sample forms a paraboloid of revolution; and

FIG. 40 shows another specific example in accordance with the present invention for observing the shape of an inner surface for an example in which the shape of the inner surface of a sample forms a paraboloid of revolution.

### BEST MODE FOR CARRYING OUT THE INVENTION

The present invention relates to shape and gradient detection and/or shape and gradient measurement, and particularly relates to an optical device capable of three-dimensional-shape measurement and to a method for extracting object information including three-dimensional information. In particular, the present invention relates to an optical device for detecting and/or measuring the shape and gradient of an observed sample and method for extracting the object information, the device and method being advantageous in shape-measuring microscopes, biological microscopes, shape-measuring telescopes, medical diagnostic devices, mammography devices, gradient sensors, and the like.

In the present invention, a technique is provided for reconstructing three-dimensional shapes or otherwise detecting and/or measuring shapes in which the polarization state in the reflected light varies because the change in amplitude and phase differ due to the p-component (the component of the direction in which the electric vector is parallel to the incident plane) and the s-component (the component of the direction in which the electric vector is perpendicular to the incident plane) of the electric vector of the light in the case that polarized light, which is light in which the electric field and the magnetic field oscillates in only one specific direction, is reflected at a material surface; and it is possible to use that fact that (1) “the orientation of the incident plane is used as a reference for change in the state of the polarized light,” and the change in the state of the polarized light is (2) “a monotone function with respect to the incident angle,” in order to measure the polarization state of the reflected light, whereby the incident angle and the orientation of the incident plane can be calculated from the measured values, the surface of a substance as the sample, and the vicinal faces, i.e., the gradients of the tangent plane reflecting the incident light can be determined, and the surface of the observed substance can be reproduced by smoothly connecting gradients of the vicinal faces thus determined because the surface of the observed substance can be made to be continuous within the observation field of view.

The present invention provides a technique capable detecting the shape and gradient and/or measuring the shape and gradient of a sample by uniformly illuminating polarized light having a controlled, already-known polarization (e.g., right circular polarized light) from the periphery onto a sample substance (e.g., transparent, spherical cells) having a smooth surface (boundary), and observing sample as a polarized light image from a spatially fixed observation direction

In a first aspect of the present invention, light beams reflected in the observation direction are each composed of a gradient-plane component specularly reflected at the gradient plane (tangent plane) that forms the surface (boundary) of a sample. A vicinal-face component is generated as a result of the reflection that satisfies the law of reflection at the vicinal face. When circular polarized light is incident, the light reflected at the surface of a transparent body becomes elliptically polarized light, the major axis of the ellipse is constantly parallel to the gradient plane (tangent plane), and the ellipticity angle of the ellipse in the elliptically polarized light is in a simple linear relationship with the incident angle. Therefore, the azimuth angle of the incident plane (normal of the tangent plane) can be determined from the orientation of the major axis and the reflection angle (equal to the incident angle by the law of reflection) can be determined from the ellipticity, by measuring the polarization state (differential ellipticity and orientation of the major axis of the ellipse). As a result, it is possible to measure the gradient angle of the reflection vicinal face in relation to the observation direction. Since the surface of a spherical cell is continuous within the observation field of view, a three-dimensional shape can be reproduced using the data to smoothly connect the gradients of vicinal faces thus determined.

Thus, the present invention is a technique that makes it possible to determine the three-dimensional shape of a sample, including the coordinates of the optical axis direction, using the reflection characteristics of the polarized light.

The present invention provides a technique for extracting shape characteristics and/or gradient characteristics by using the fact that change in the polarization state that occurs due to reflection of polarized light at the surface of an observed object is dependent on the incident angle; calculating the angle formed by the axis in the observation direction and the normal of the tangent plane at the point of reflection at the surface of the observed object and the polarization angle of the component projected onto the plane perpendicular to the axis in the observation direction; obtaining the partial derivative coefficient at the reflection point of the object from the slope of the measured normal line; and making use the measured values of the temporal and spatial changes in the partial derivative coefficient. The present invention provides a technique for constructing a three-dimensional shape by integrating the measured partial derivative coefficient in the entire region of the observed surface. The present invention also provides a technique for detecting and/or measuring by extracting the physical optical characteristics of the reflection surface in view of the fact that the geometric shape does not depend on the observation wavelength.

The present invention also provides a simple method that can be generally used for detecting shapes/gradients, and/or measuring and analyzing shapes/gradients in which certain properties and a special configuration are used in combination, the certain properties being the two properties shared by all substances in relation to light, i.e., (a) the complex amplitude reflectance ratio ρ is −1 at an incident angle φ of 0 and a radian of 0°, and is 1 when φ=π/2 and radians=90°, and (b) a complex quantity ρ varies monotonically from −1 to 1 on a complex plane when the incident angle φ varies from 0 to π/2 and invariably passes through an imaginary axis (Δ=±π/2) at a midway point in which the real part of ρ is 0; and the special configuration being a combination of configurations in which a sample is uniformly illuminated with circular polarized light from the periphery of the sample, and the state of the specularly reflected polarized light is observed from a spatially fixed direction, whereby the incident angle (=reflection angle) and the slope of the incident plane at the reflection point are measured from the shape of the reflected elliptic polarized light observed at a predetermined reflection point on the cross-sectional coordinate of a sample, and the shape of the sample is reconstructed by sequentially and smoothly connecting the measured reflection surfaces of the reflection points between measurement points in a sample cross section.

The present invention provides an optical device for shape and gradient detection and/or measurement and a method for extracting object information, the device and method being used for implementing the technique described above.

(Principles of the Present Invention)

The polarization measurement used in the present invention is related to ellipsometry, which is a conventional method for precisely analyzing polarization. Ellipsometry is long-known as a method for precisely measuring the thickness of a thin film sample, precisely measuring the optical properties of the surface of a sample, or otherwise measuring the optical properties of a sample using the properties of polarization, which is fundamental characteristic of light (electromagnetic waves in general) reflected at the surface of an object.

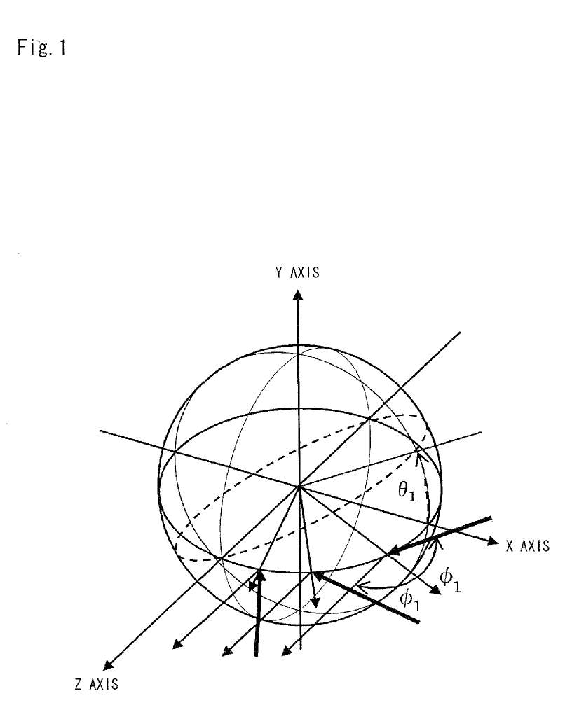

On the other hand, the principles for measuring the gradient angle of the surface of an object according to the present invention provide a new concept referred to as “gradient ellipsometry,” in which ellipsometry is used for precisely measuring geometric shapes, and has never been previously used prior art. Perfectly polarized light in a specific polarization state (e.g., right circular polarized light) is uniformly illuminated from the periphery onto a sample object (e.g., a transparent spherical cell) having a smooth surface or boundary for the purpose of measuring the shape of a sample, as shown in FIG. 1. The sample is observed as a polarized light image from a spatially fixed observation direction. When the observation direction is the z direction, light beams reflected in the z direction are each composed of a specularly reflected vicinal face component at the vicinal face (tangent plane) that forms the surface (boundary) of a sample. This “vicinal face component” is produced as a “result of reflection that satisfies the law of reflection at a vicinal face.” Here, with actual reflection at the surface of an object, the reflected light is generally partially polarized light in the case that the object has a surface roughness of a magnitude that cannot be ignored in relation to the wavelength of the light. However, all partially polarized light is described to be the sum of the perfectly polarized light component and the unpolarized light component, and in ellipsometry, already-known perfectly polarized light is made incident and the state of the polarized light of the reflected perfectly polarized light component is the target of measurement. The unpolarized light component is measured as needed as the degree of polarization.

Considered first is the treatment of perfectly polarized light in standard ellipsometry.

The reflection of a vicinal face can be defined by the slope of the normal of the tangent plane because the observation direction is fixed.

For example, this can be expressed as the angle φ₁ formed by the z-axis, which is the observation direction and the rotational angle θ₁ from the x-axis using the z-axis as the axis of rotation, wherein θ₁ is equal to the angle of deviation of the component projected onto the x-y plane of the normal of the tangent plane.

Using reflection within an x-y plane having an angle of deviation of 0° as an example in the spherical sample illustrated in FIG. 1, the law of reflection is satisfied in the case that incident light beams are incident in the manner indicated by the bold lines at their respective reflection points, and the reflected light beams are parallel to the z-axis. It is apparent from FIG. 1 that the slopes φ₁ and θ₁ of the normal of the tangent plane are equal to the incident angle of the light beams and the angle of deviation of the incident plane, respectively.

The reflection characteristics can be described in terms of the complex amplitude reflectance because the amplitude and phase of light generally changes with the reflection of the light at the surface of an object. When consideration is given to the polarization of light, the complex amplitude reflectance assumes different values in the p component, which is the component of the polarization within the incident plane (defined by the plane containing the normal of the reflection surface and the incident light beams) and the s component perpendicular to the incident plane (parallel to the surface).

[symbol]

[Formula 1]

rₚ is the complex amplitude reflectance of the p component

rₛ is the complex amplitude reflectance of the s component

When the incident light is expressed in the preceding manner, the amplitude and phase of the p component and the s component of the electric vector of light vary in a different manner when the polarized light, which is light having polarization, is reflected by the surface of an object. Therefore, the state of the polarized light changes in the reflected light and generally becomes elliptically polarized light.

Elliptically polarized light can be expressed as two real variables. When the two variables are the azimuth angle of the major axis and the ellipticity of the ellipse, “the reference of variation in the azimuth angle of the major axis is the p-direction, and matches the orientation of the incident plane that includes the normal of the tangent plane.” Variation in ellipticity is “a monotone function in relation to the incident angle.” The gradient ellipsometry of the present invention uses these two properties to determine the gradient of a vicinal face at the reflection point of light using a step 1 in which the polarized light ellipse of the reflected perfectly polarized light component is measured and the orientation of the incident plane is determined from the measured value of the azimuth angle of the ellipse major axis, and a step 2 in which the theoretical value of the incident angle dependency is used and calculated from the measured value of ellipticity of the ellipse.

In other words, in the present invention, perfectly polarized light having a controlled, already-known polarization (e.g., right circular polarized light) is uniformly illuminated from the periphery onto a sample object (e.g., a transparent spherical cell) having a smooth surface (boundary) in order to detect the shape and gradient and/or measure the shape and gradient of the sample. The sample is observed as a polarized light image from a spatially fixed observation direction.

In this configuration, the light beams reflected in the observation direction are each composed of a vicinal face component specularly reflected at the vicinal face (tangent plane) that forms the surface (boundary) of the sample, as shown in FIG. 1. The vicinal face component is generated as a result of reflection that satisfies the law of reflection at the vicinal face.

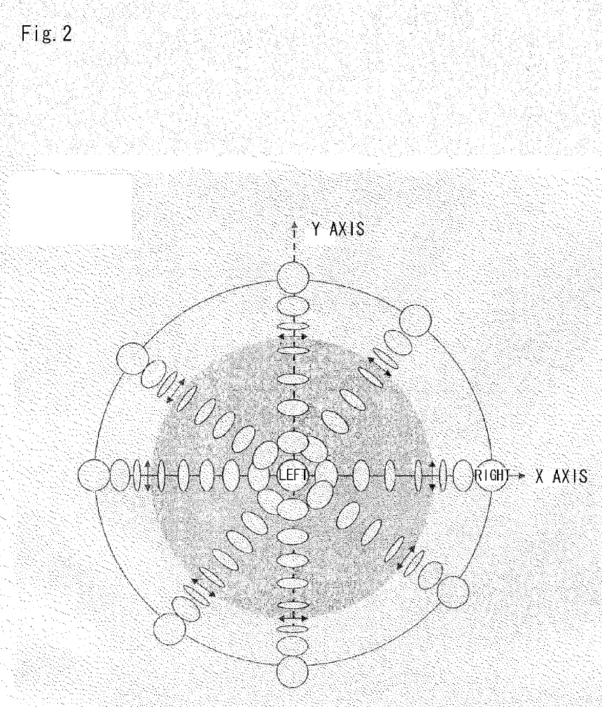

The specific case of illumination with circularly polarized light will be considered. Unless otherwise noted, the polarized light is perfectly polarized light with a single fixed shape of a polarized light ellipsis. A group elliptically polarized light beams such as that shown in FIG. 2 is formed when the polarized light ellipses of the light reflected at the surface of a transparent body are calculated with the specific condition that circular polarized light is incident. The term “left” in the center of FIG. 2 indicates that left circular polarized light is reflected and the term “right” at the periphery indicates that right circular polarized light is reflect. With perpendicular incidence at φ=0°, the rotational direction of the polarized light is reversed because the traveling direction of the light is reversed due to reflection, and the incident right circular polarized light is reflected as left circular polarized light. With grazing-incidence at φ=90°, the polarization state of the incident light does not vary from the continuity of physical phenomenon and is reflected in an unchanged polarization state. These facts correspond to boundary conditions related to the incident angle φ and are derived from the spatial geometric properties. Therefore, the facts hold true with transparent bodies as well as absorbing bodies and do not depend on the substance of the sample.

In the case that right or left circular polarized light is incident, the incident angle changed from the continuity of physical phenomenon, whereby the state of reflected polarized light changes for all substances in a continuous fashion from left (right) circular polarized light to right (left) circular polarized light, and invariably passes through a state of linear polarization at an intermediate point. Therefore, the angle monotonically increases from −45° to +45° in terms of ellipticity angle of the ellipse. In other words, with circular polarized light incidence, the range of variation in the incident angle matches the variable range of the polarization state, and maximum sensitivity is assured. A negative ellipticity angle indicates left polarized light and a positive ellipticity angle indicates right polarized light.

The group of polarized light ellipses of FIG. 2 schematically show that the major axes of the ellipses are constantly parallel to the vicinal face (tangent plane) and that the ellipticity angle varies with the incident angle because the later-described amplitude reflection coefficient is a real number in a transparent body. Therefore, the azimuth angle of the incident plane (normal of the tangent plane) can be determined from the orientation of the major axis and the reflection angle (equal to the incident angle in accordance with the law of reflection) can be determined from the ellipticity by calculating the polarization state (the direction of the major axis and the ellipticity of the ellipse). In this manner, the gradient angle of the reflection vicinal face in relation to the observation direction can be precisely measured.

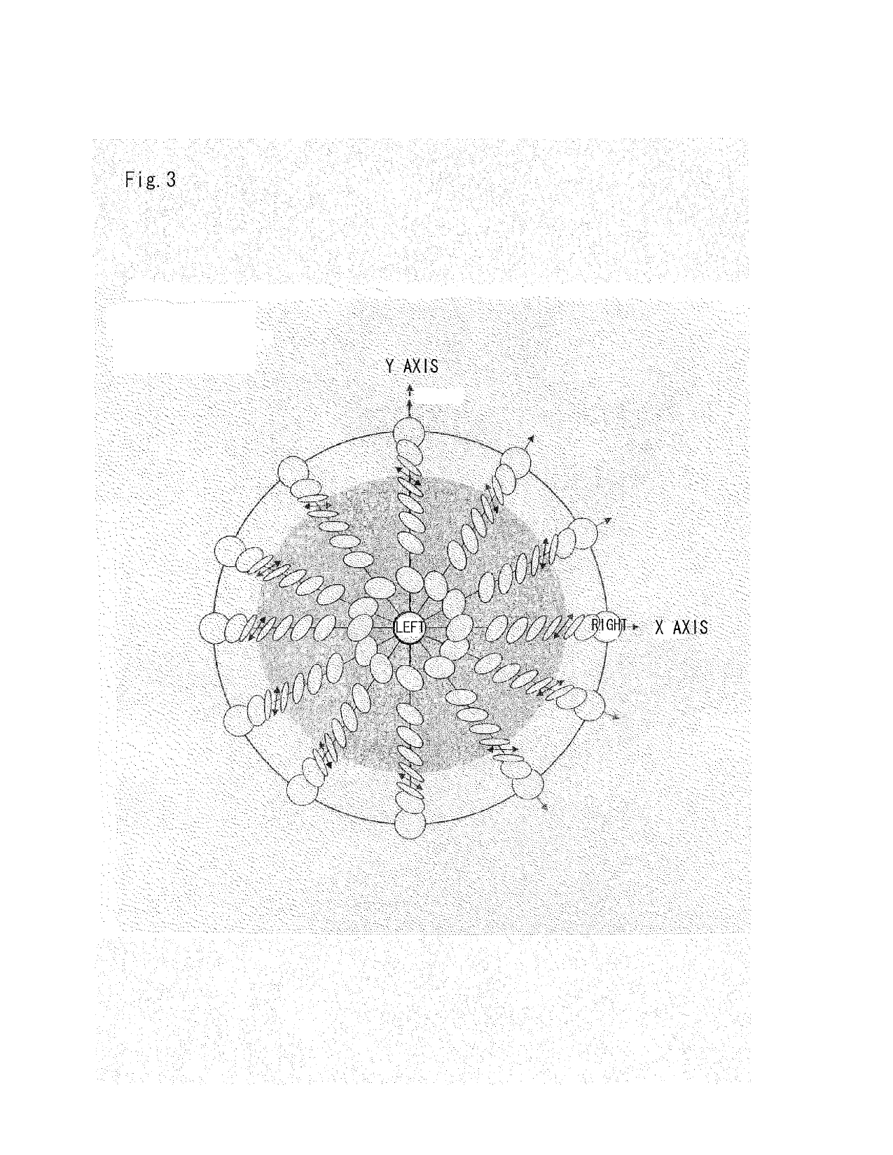

In the description of the present specification below, variation in the polarization state due to reflection will be described with computational examples as necessary for the cases of a transparent spherical cell and an absorbing metallic luster sphere. The law of reflection of FIG. 1 holds true even with an absorptive sample. However, the incident angle dependency of the polarization state varies from FIG. 2 in the case of an absorptive sample, and the azimuth angle of the ellipse is rotated 45°, as shown in FIG. 3, with a sphere whose surface is covered with Al, for example. This rotation systematically occurs by a predetermined distance for all reflection. Therefore, a fixed offset in angle of deviation is produced about the z-axis when the gradient angle of the vicinal face is calculated. Analytic theory of ellipsometry can be applied in order to ascertain the variation in the polarization state depending on the material of the reflection surface. The surface is continuous within the observation field of view for typically-shaped samples as well as spherical samples, so a three-dimensional shape can be reproduced by smoothly connecting the gradients of the vicinal face thus determined.

The principles of measuring surface gradients described in the present invention hold true for electromagnetic waves of all wavelengths. Also, the light that is used may be white light, including light from the UV, visible, and infrared light regions to the microwave region and the like, and may be laser or another monochromatic light. The object surface is considered to be smooth to the extent that specular reflection occurs, but the reflectivity is sufficient as long as the reflected light can be detected by a detector, and in terms of the human body or the like, the light may be infrared to the microwave region. In other words, the precision of shape measurement can be improved by carrying out measurements in conditions in which a scatter component is not generated from the surface and the reflected light is perfectly polarized light, and such conditions can be obtained by using longer wavelengths in comparison with the roughness of the object surface.

The object is placed at the origin of a right hand coordinate system (x, y, z), wherein z is the observation direction of the reflected light. The point of observation is considered to be essentially z=∞ because observation is carried out at a sufficient distance away in comparison with the size of the object. Observation is carried out on the x-y plane positioned at a sufficient distance away and the image formation relationship of a predetermined magnification (e.g., known magnification) is maintained. The reflected light beams are parallel to the z-axis, and the projection component (x₁, y₁) of the x-y plane at the point of reflection is known from the coordinates of the reflected light beams in the x-y plane.

The coordinate z₁ of the z-axis component in the depth direction of the object at the reflection point can be determined in order to measure the smooth surface of an object. In ordinary microscopic observation, the coordinate z₁ cannot be established. With gradient ellipsometry of the present invention, the partial derivative coefficient at z₁ can be established as the gradient of the tangent plane of the reflection point (x₁, y₁, z₁) using the reflection characteristics of the polarized light.

Generally, in image formation systems for microscopic measurement and the like, observation can be carried out in a state in which the surface of a substance is sufficiently smooth by selecting the magnification and observation direction. Therefore, z₁ can be sequentially established by an integrating operation for smoothly connecting vicinal faces that constitute the tangent planes.

The measurement data of gradient ellipsometry of the present invention can be variously used as precision data for gradients and temporal changes in gradients. Furthermore, in the case that a three-dimensional shape of a sample is to be reconstructed from gradient data, it is possible to use as the reconstruction algorithm a three-dimensional measurement algorithm for transparent body shapes used in the field of robotics. It is possible to make use of research and development that uses a measurement of degrees of polarization of the reflection of a transparent object under unpolarized light illumination, and more particularly it is possible to use research related algorithms for reconstructing an object shape from the normal of the tangent plane of a transparent object (D. Miyazaki, M. Saito, Y. Sato, K. Ikeuchi, “Determining surface orientations of transparent objects based on polarization degrees in visible and infrared wavelengths,” J. Opt. Soc. Am. A, 19(4), pp. 687-694, 2002; D. Miyazaki, R. T. Tan, K. Hara, K. Ikeuchi, “Polarization-based Inverse Rendering from a Single View,” Proc. IEEE Intl. Conf. Computer Vision, 2003. pp. 982-987, 2003; Daisuke Miyazaki, Katsushi Ikeuchi, “Inverse Polarization Raytracing: Estimating Surface Shape of Transparent Objects,” in Proceedings of International Conference on Computer Vision and Pattern Recognition, San Diego, Calif. USA, 2005.06; D. Miyazaki, K. Ikeuchi, “A Method to Estimate Surface Shape of Transparent Objects by Using Polarization Raytracing Method,” The Transactions of the Institute of Electronics, Information and Communication Engineers D-II, Vol. J88-D-II, No. 8, pp. 1432-1439, 2005.08).

The coordinates for starting connections are arbitrary, and connections can be made, e.g., starting from the center of an observation screen and widen out to the periphery. In other words, in relation to the z coordinate in the center of the screen, the shape can be determined as long as the average value can be determined.

The observation surface of a spherical sample forms a circle, and the polarization state of the light at the observation point inside the circle forms the state shown in FIG. 2, and the major axis of the ellipse is orthogonal to the incident plane. The ellipticity ∈ varies in continuous fashion as a function of incident angle from right circular polarized light at s=1 with grazing reflection at φ=90° to left circular polarized light at ∈=−1 with reflection at perpendicular incidence at φ=0°.

At a polarization angle (Brewster's angle φ_{B}=tan⁻¹ n) of a medium having a refractive index of n=1.5 in the vicinity of φ=56°, ellipticity passes through linearly polarized light at ∈=0. At the center of FIG. 2, a left-polarized light group is observed in a region with an incident angle of less than φ_{B} (inner side of linearly polarized light; indicated by the shaded circle inside the circle of the contour line of the sample of FIG. 2), and a right polarized light group is observed in the peripheral portion of the sample outside of the shading shown in FIG. 2. The incident angle φ₁ can be measured from the ellipticity ∈₁, and the polarization angle θ₁ of the incident plane can be measured from the azimuth angle of the ellipse.

FIG. 2 illustrates the case of a spherical sample. In the case that the sample is another common shape, the two-dimensional distribution of the observed ellipses shown in FIG. 2 varies so that an analogy can be readily made. However, in this case as well, the shape of the observed ellipses and the gradient of the vicinal faces of the reflection points thereof have a 1:1 correspondence, variation in the observed elliptical polarized light is also continuous because the surface of the sample is smooth, and general applicability is not lost.

The law of reflection of FIG. 1 holds true even when the sample is absorbing body.

However, the incident angle dependency of the polarization state varies from FIG. 2 in the case of an absorptive sample, and the azimuth angle of the ellipse is rotated 45°, as shown in FIG. 3, with a sphere whose surface is covered with Al, for example. This rotation systematically occurs by a predetermined distance for all reflection. Therefore, a fixed offset in angle of deviation is produced about the z-axis when the gradient angle of the vicinal face is calculated. Analytic theory of ellipsometry can be applied in order to ascertain the variation in the polarization state depending on the material of the reflection surface. Hence, the surface shape can be reconstructed using the same method.

Variation in the polarization state due to reflection can be formulated in the following manner by the complex refractive index of the substance.

The observed polarization state of reflection can be expressed by the ratio of p-s components of the complex amplitude reflectance at the reflection point of the sample (“Henko Sokutei to Henko Kaisekiho” (Measurement and Analysis of Polarized Light) by Masaki Yamamoto; Hikari Kogaku Handbook” (Handbook of Optical Engineering) by Teruji Kose, et al., Asakura Shoten, 1986, pp. 411-427 (Non-patent Document 4).

$$\frac{{r}_{p}}{{r}_{s}} ≡ ρ = tan ψ \cdot exp ( i Δ ) \vert \qquad [ Formula 2 ]$$

The complex variable ρ is an ellipsometric (ellipsometry) parameter (“Hikari Kougaku Handbook” (Handbook of Optical Engineering) by Teruji Kose, et al., Asakura Shoten, 1986, pp. 411-427 (Non-patent Document 4), see formula (2.5.38) in particular, and is expressed as a complex amplitude reflectance ratio ρ in Non-patent Document 2), and the actually measured real variables Ψ and Δ correspond to the expression of the complex variable ρ in polar coordinates.

The light polarization state is expressed by Jones' vector having a horizontal component Ex and a vertical component Ey of the electric vector of light.

$$[ \begin{matrix}{E}_{x} \\ {E}_{y}\end{matrix} ] \vert \qquad [ Formula 3 ]$$

When Jones' computations are used, variation in the polarization state can be expressed in the following manner.

Right circularly polarized light

$$\frac{1}{\sqrt{2}} [ \begin{matrix}- i \\ 1\end{matrix} ] \vert \qquad [ Formula 4 ]$$

is incident on the tangent plane of the slopes φ and θ of the normal line, and is specularly reflected at the surface of the sample.

Assuming that

[symbol]

[Formula 5]

rₚ|_{φ=φ}_{₁} is the complex amplitude reflectance of the p component, and

rₛ|_{φ=φ}_{₁} is the complex amplitude reflectance of the s component

at the time the incident angle φ=φ₁ in the reflection from the sample, the reflected light

$$[ \begin{matrix}{E}_{ox} \\ {E}_{oy}\end{matrix} ] \qquad [ Formula 6 ]$$

can be expressed in the following manner, assuming that the intensity normalization coefficient

$$\frac{1}{\sqrt{2}} \qquad [ Formula 7 ]$$

can be ignored.

$$[ \begin{matrix}{E}_{ox} \\ {E}_{oy}\end{matrix} ] = {T}_{{θ}_{1}} {R}_{{φ}_{1}} {T}_{- {θ}_{1}} [ \begin{matrix}- i \\ 1\end{matrix} ] \qquad [ Formula 8 ]$$

Here,

$${T}_{θ} = [ \begin{matrix}cos θ & - sin θ \\ sin θ & cos θ\end{matrix} ] \qquad [ Formula 9 ]$$

is a rotator matrix at an angle θ that accompanies the rotation of the coordinate system, and

$${R}_{{φ}_{1}} = {r}_{s} {\vert}_{φ = {φ}_{1}} \cdot ⌈ \begin{matrix}{ρ}_{1} & 0 \\ 0 & 1\end{matrix} ⌉ \qquad [ Formula 10 ]$$

is a Jones' Matrix that expresses the reflection of polarized light at an incident angle φ₁ on a vertical sample surface with a horizontal incident plane. The ellipsometric parameters (see formula (2.5.38) of Non-patent Document 2 noted above) can be expressed as

$${ρ}_{1} ≡ {\frac{{r}_{p}}{{r}_{s}} \vert}_{φ = {φ}_{2}} = tan {ψ}_{1} \cdot exp ( {iΔ}_{1} ) \qquad [ Formula 11 ]$$

Circular polarized light does not vary as

T_{-θ} [Formula 12]

and is therefore

$$[ \begin{matrix}{E}_{ox} \\ {E}_{oy}\end{matrix} ] = {T}_{- {θ}_{1}} [ \begin{matrix}- {iρ}_{1} \\ 1\end{matrix} ] \qquad [ Formula 13 ]$$

In the case of a transparent sample, ρ₁ is

ρ₁=±tan ψ₁ [Formula 14]

a real number, the slope of the ellipse equates to θ₁. Step 1 is a simple direct reading. In step 2, the ellipticity equates to ρ₁, and the incident angle can be determined from the theoretically calculated value of the ratio of later-described Fresnel amplitude reflection coefficients using the refractive index of the sample.

In cases in which the sample is a metal, ρ₁ is generally a complex number. Here, ellipsometric techniques will be employed while noting that the reference for theoretical calculation is the p-direction, which is the direction of the incident plane (see Measurement and Analysis of Polarized Light) by Masaki Yamamoto; “Hikari Kogaku Handbook” (Handbook of Optical Engineering) by Teruji Kose, et al., Asakura Shoten, 1986, pp. 420 (Non-patent Document 4), and particularly formula (2.5.36) disclosed therein). The ratio of Jones' vector components,

$${ξ}_{o} = \frac{{E}_{oy}}{{E}_{ox}} = \frac{i}{{ρ}_{1}} = \frac{i}{tan {ψ}_{1} \cdot exp ( {iΔ}_{1} )} \qquad [ Formula 15 ]$$

is calculated using the p-direction of the reflected elliptical polarized light is used as a reference, Ψ₁, Δ₁ are calculated in accordance with the theoretical formula of the ratio of later-described Fresnel amplitude reflection coefficients using the complex refractive index of the sample, and the azimuth angle of the major axis of the polarized light ellipse to be observed and the theoretical value of ellipticity are calculated. These are monotonic functions in relation to the incident angle.

Since the ellipticity angle of the ellipse and azimuth angle of the major axis are linked in a simple relationship with Ψ₁, Δ₁, mutual conversion is simple.

Thus, the incident angle that determines the theoretical ellipticity equating to the measured ellipticity is uniquely determined by step 2 in the case of an absorptive sample. The azimuth angle of the incident plane of step 1 is determined from the theoretical value of the azimuth angle of the major axis of the polarized light ellipse in the incident angle. The measurement precision is 0.01° to 0.001° with Ψ, Δ using ordinary ellipsometric techniques in which the polarization state of the perfectly polarized light is the target of measurement. The measured Ψ₁, Δ₁ are both functions of the incident angle φ₁ to the sample, and the measurement precision of the incident angle can also reach an equivalent 0.01° to 0.001° as shown next.

Actually, in ellipsometry, a principal angle of incidence method is known and used for measuring the principal angle of incidence Φ_{P} defined when Δ=±π/2, and the principal azimuth angle ψ_{P} defined as ψ at the principal angle of incidence, in similar fashion to the method for directly measuring Ψ, Δ of a sample (K. Kinosita and M. Yamamoto, Principal Angle of Incidence Ellipsometry, Surface Sci., 56, 64-75 (1976); Masaki Yamamoto, “A Note on Ellipsometric Measurements on Silicon Surfaces,” Oyo Butsuri, 50, 777-781 (1981)), and the principal angle of incidence can be established with a precision of 0.01° to 0.001° (M. Yamamoto and 0. S. Heavens, A Vacuum Automatic Ellipsometer for Principal Angle of Incidence Measurement, Surface Sci., 96, 202-216 (1980)). These incident angle measurement sensitivities are 1/6000 to 1/60000 when converted to the gradient angle of the sample surface, and correspond to a sensitivity to changes in the concavo-convex gradient in units of 1 μm and 0.1 μm in terms of the difference in height at the two ends of a width of 6 mm.

Described next are the calculations for the complex amplitude reflectance ratio.

The incident angle dependency of the reflected polarized light can be known by calculating complex amplitude reflectance ratio ρ using a suitable optical model that expresses the optical properties of the sample. A bulk sample can be described in a simple ratio of Fresnel amplitude reflection coefficients, and the same formula can be used for a transparent body and an absorptive body.

$$tan {ψ}_{1} \cdot exp ( {iΔ}_{1} ) = - \frac{cos ( {φ}_{1} + {φ}_{1}^{′} )}{cos ( {φ}_{1} - {φ}_{1}^{′} )} \qquad [ Formula 16 ]$$

However,

φ′ [Formula 17]

is the complex refractive angle and is bound by Snell's law

nₐ sin φ=ñ sin φ′| [Formula 18]

and the complex refractive angle of the bulk form is a function of

ñ=n−ik. [Formula 19]

Also,

nₐ [Formula 20]

is the refractive index of a medium, in the visible light region, is equal to 1 in air and about 1.33 in water.

$$tan {ψ}_{1} \cdot exp ( {iΔ}_{1} ) = - \frac{cos ( {φ}_{1} + {φ}_{1}^{′} )}{cos ( {φ}_{1} - {φ}_{1}^{′} )} \qquad [ Formula 21 ]$$

The function above is a simple function in relation to the incident angle and can be readily calibrated for each substance.

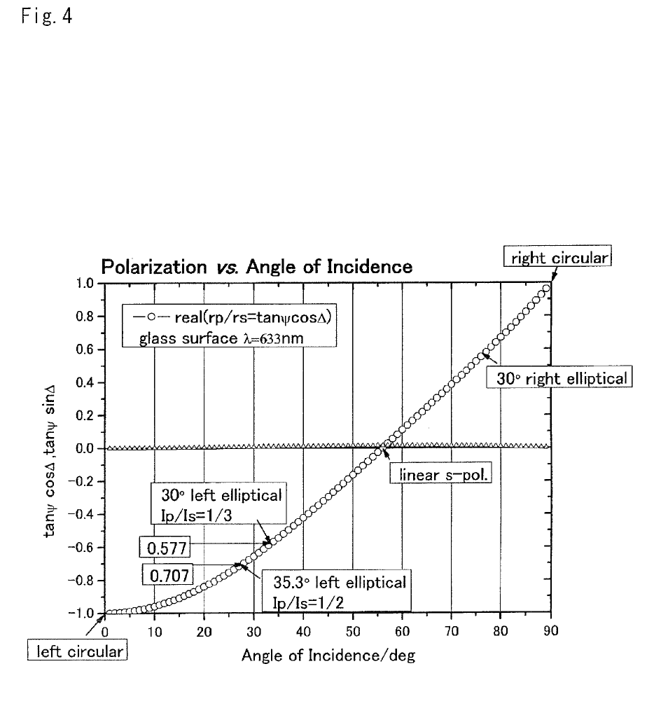

In FIG. 4, a calculation example is shown for the case of glass having a refractive index of n=1.5 in air in order to describe variation in relation to a transparent sample. With a transparent substance, the imaginary number part of ρ is constantly 0 because the extinction coefficient k=0 in a transparent substance. The real number part monotonic varies from −1 to 1 as shown in the diagram when the incident angle φ increases from 0° to 90°.

When illuminated with right circularly polarized light, the polarization state changes from left circularly polarized light to right circularly polarized light, and passes through linearly polarized light at an intermediate point of the polarization angle. Therefore, in terms of the ellipticity angle of the ellipse, the increase is monotonic from −45° to +45°. A negative ellipticity angle indicates left polarized light and a positive ellipticity angle indicates right polarized light.

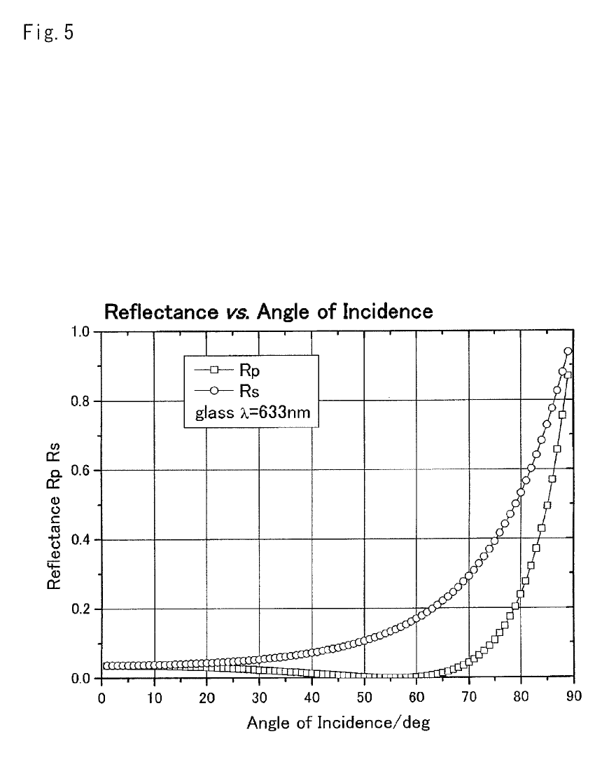

FIG. 5 shows the incident angle dependency of the intensity reflectance of the p-s polarized light components in the sample shown in FIG. 4.

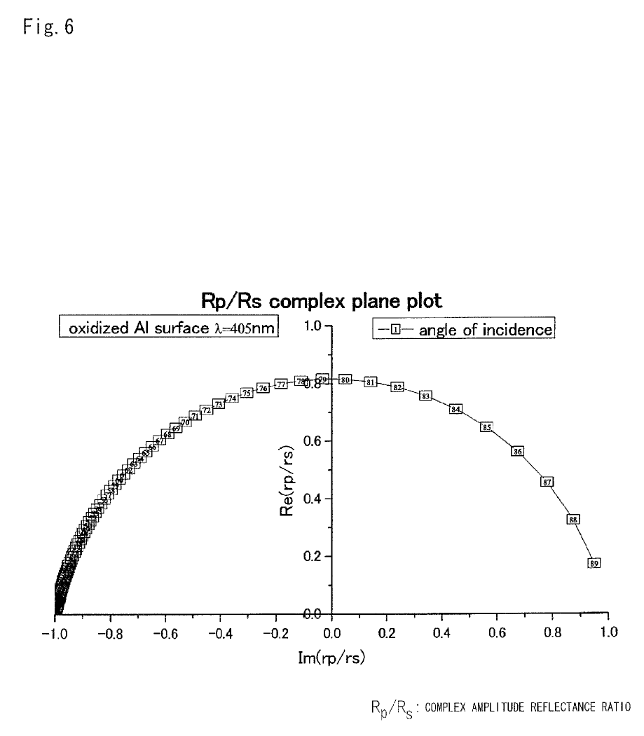

FIG. 6 shows the incident angle dependency of ρ of an Al sample at a wavelength of 405 nm in a complex planar display as an example of the incident angle dependency in an absorptive sample. Since the sample is absorptive, the complex refractive index is

ñ=n−ik [Formula 22]

0.6-5.04i.

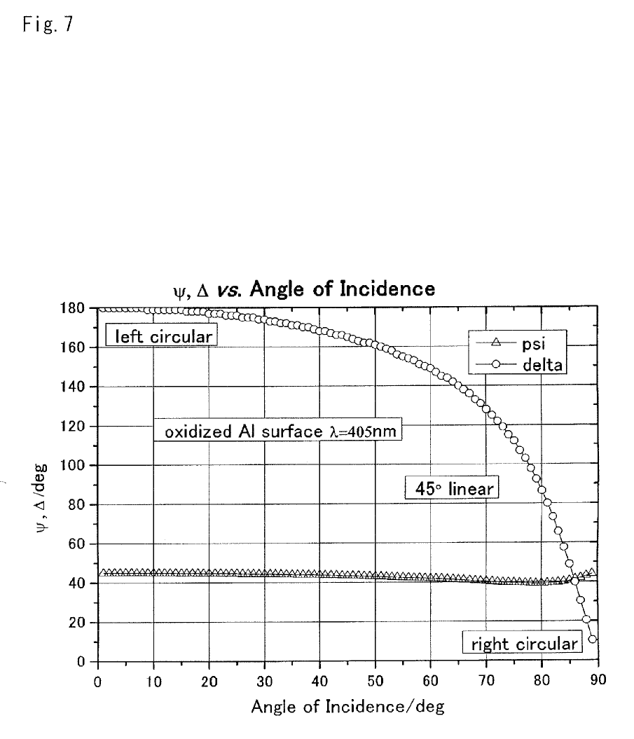

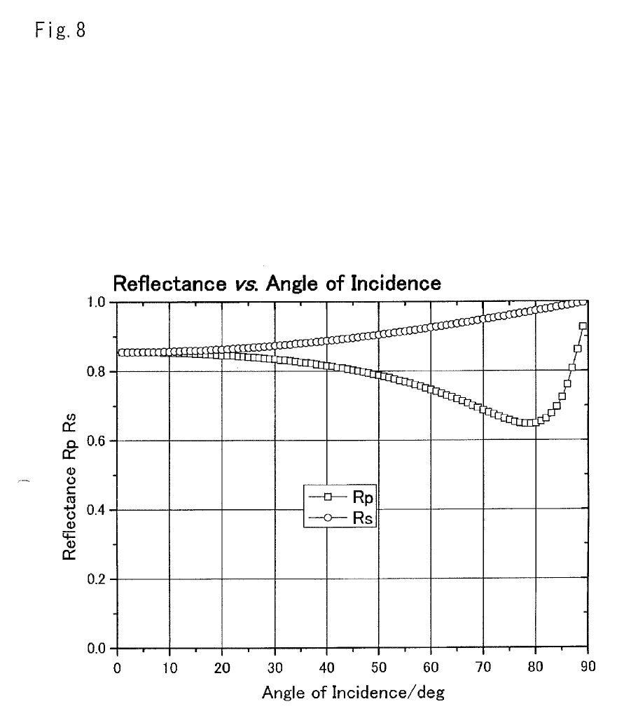

The monotonic variation from −1 to 1 is the same as for a transparent body when the incident angle φ increases from 0° to 90°, as shown in the diagram. However, the imaginary number part does not reach 0 because Al is absorptive, and the variation midway is considerably different. In the case of Al, Ψ and Δ behave in a characteristic manner, as shown in FIG. 7, with variation along a circle having a radius of 1. The reflectivity of both p- and s-polarized light is high and Ψ is substantially constant between 40° and 45°, which can be seen from the change in intensity reflectance shown in FIG. 8. The maximum value of Ψ may be considered to be 45° because of a shared properties of substances, which is that the reflectance of s-polarized light is greater than the reflectance of p-polarized light.

Therefore, Δ undergoes a large variation from 180° (corresponding to −1 on the complex plane of FIG. 6) to 0° (corresponding to +1 on the complex plane of FIG. 6) with metallic bodies or the like that are highly absorptive, as shown in FIG. 7. The following points are the exactly same for a transparent body, that is, when right circularly polarized light is illuminated, the polarization state varies from left circularly polarized light to right circularly polarized light as shown in the boxes of FIG. 7, and passes through linearly polarized light at the principal incident angle of Δ=±90° (on the real axis of FIG. 6). However, all observed polarized light is inclined from the incident plane by an angle equal to the azimuth angle equal to T. In the example of FIG. 7, is constantly inclined substantially 45°, and the ellipse at an intermediate point and the linearly polarized light uniformly inclined 45° (an inclined circle is still a circle), as shown in FIG. 3.

$$\begin{matrix}tan {ψ}_{1} \cdot exp ( {iΔ}_{1} ) = - \frac{cos ( {φ}_{1} + {φ}_{1}^{′} )}{cos ( {φ}_{1} - {φ}_{1}^{′} )} & [ Formula 23 ] \\ {n}_{a} sin φ = \tilde{n} sin {φ}^{′} \vert & [ Formula 24 ]\end{matrix}$$

The formulas above express the case in which the surface of a sample substance is considered to be in bulk form.

The method of the present invention is also effective in cases in which the surface is covered with, e.g., an oxide film, a cell membrane, or the like, in the case that the surface of the sample is not in bulk form. In this case, the reflection surface can be optically modeled to calculate the complex variable ρ and can be correlated with the variation in the observed polarization state. The methodology implemented in conventional ellipsometry can be used without modification in the analysis and calculation portion. In other words, as long as light is reflected and variation in the polarization state of the perfectly polarized light component can be observed, ellipsometric techniques can be used regardless of how the observation values are used and the information that is extracted.

A description of ellipsometric techniques can be found in, e.g., H. G. Tompkins and W. A. McGahan, Spectroscopic Ellipsometry and Reflectometry: A User's Guide, John Wiley & Sons, New York, 1999; H. G. Tompkins, A User's Guide to Ellipsometry, Academic Press, San Diego, 1993; R. M. A. Azzam and N. M. Bashara, Ellipsometry and Polarized Light, North Holland Press, Amsterdam, 1977, Second Edition, 1987; R. M. A. Azzam, Selected Papers on Ellipsometry, SPIE Milestone Series MS27, 1991; Spectroscopic Ellipsometry, A. C. Boccara, C. Pickering, J. Rivory, eds., Elsevier Publishing, Amsterdam, 1993; and “Bunko Eripusometori” (Spectroscopic Ellipsometry) by Hiroyuki Fujiwara, Maruzen, (2003) (ISBN 978-4-621-07253-0). The cited literature and content thereof are included in the disclosure of the present specification.

In this manner, it is possible to directly and precisely measure the angle φ₁ formed by with the z-axis, which is the observation direction, and the polarization angle ƒ₁ of the projection component to the x-y plane, in relation to the normal of the tangent plane at the reflection point (x₁, y₁, z₁) of the observed object surface from the incident angle dependency of the variation in the polarization state that occurs with an single reflection. The slope of the measured normal line gives the partial derivative coefficient at the reflection point (x₁, y₁, z₁) of the object. In accordance with the gradient ellipsometry of the present invention, the shape characteristics and/or the gradient characteristics can be extracted by directly using the measured values of the temporal and spatial changes of the partial derivative coefficient.

It is readily apparent that the construction of a three-dimensional shape can be achieved by integrating the measured partial derivative coefficient in the region of the entire observation plane. In view of the fact that a geometric shape does not depend on the observation wavelength, and the physical optical characteristics of the reflection surface can be extracted and measured. It is also possible to apply research known and implemented in the field of robotics applications (e.g., D. Miyazaki, M. Saito, Y. Sato, K. Ikeuchi, “Determining surface orientations of transparent objects based on polarization degrees in visible and infrared wavelengths,” J. Opt. Soc. Am. A, 19(4), pp. 687-694, 2002).

It was described above that the orientation of the incident plane can be detected from the theoretical value of observed azimuth angle of the polarized light ellipse as a step 1, in which the orientation of the incident plane is detected from the observed azimuth angle of the polarized light ellipse of the perfectly polarized light of the group of light beams reflected at the object surface and emitted at a specific azimuth angle. For example, it is possible to use a device that can cause right circularly polarized light or left circularly polarized light to be incident in a switching fashion, as described below. In other words, an illumination device for causing light in a substantially already-known state of perfect polarization to be uniformly incident around the periphery of an object may be one in which the orientation of the incident plane is detected by causing right circularly polarized light and left circularly polarized light to be incident in a switching fashion and using the fact that the theoretical value of the observed azimuth angle of the reflected polarized light ellipse switches in symmetric fashion to the incident plane regardless of the reflection optical characteristics of the surface of the object.

When the present invention is used with a sample whose surface is covered by an oxide film, a cell membrane, or the like, it is possible to readily detect, e.g., variation in the thickness of a film or foreign matter deposited on the surface. Therefore, a possible application is a monitor device or the like for detecting defective shapes and foreign matter in a manufacturing line.

In the present invention, there is provided an optical device for shape and gradient detection and/or measurement to detect and/or measure the shape and gradient of a surface of an observed object using the reflection optical characteristics of the surface of an object. The optical device for shape and gradient detection and/or measurement, comprises an illumination device for causing light surrounding the periphery of the object to be uniformly incident, the light being in a polarized state which includes a substantially already-known perfectly polarized state (e.g., light in a state of perfect polarization); and a polarized light image detection device for detecting the polarization state (e.g., a polarized light ellipse of a perfectly polarized light component of a group of light beams) of a group of light beams specularly reflected by the object surface and emitted at a specific azimuth angle, wherein a gradient angle with respect to the radiated light beam of the reflection surface is measured by step 1 in which the orientation of the incident plane is detected from the observed azimuth angle of the polarized light ellipse for the refection surface of the object that forms an incident point for each reflected and radiated light beam, and step 2 in which the incident angle is detected from the ellipticity value of the polarized light ellipse, which includes the theoretical ellipticity value of the polarized light ellipse. More specifically, the gradient angle with respect to the light beams emitted from the reflection surface can be measured by detecting the orientation of the incident plane from the azimuth angle of the polarized light ellipse for the refection surface of the object that forms an incident point for each reflected and radiated light beam, and by detecting the incident angle from the ellipticity value of the polarized light ellipse. In lieu of step 1 in which the orientation of the incident plane is detected from the theoretical value of the observed azimuth angle of the polarized light ellipse, it is possible to use an illumination device for causing light in a substantially already-known state of perfect polarization to be uniformly incident around the periphery of an object, wherein the orientation of the incident plane is detected by causing right circularly polarized light and left circularly polarized light to be incident in a switching fashion and using the fact that the theoretical value of the observed azimuth angle of the reflected polarized light ellipse switches in symmetric fashion to the incident plane regardless of the reflection optical characteristics of the surface of the object. The device of the present invention can be configured as a reduction optical system including a telescope, a camera, or the like; or as a magnifying optical system for micro-measurement or the like.

In the an optical device for shape and gradient detection and/or measurement, the polarized light image detection device for detecting the polarization state of a group of light beams specularly reflected by the object surface and emitted at a specific azimuth angle may have a mechanism for specifying light beam positions on the object surface by, e.g., obtaining a reduced projection image of an object or a magnified projection image of an object, or may have a mechanism for specifying light beam positions of the object surface by providing a collimator and/or a pinhole. The polarized light image detection device for detecting the polarization state of a group of light beams specularly reflected by the object surface and emitted at a specific azimuth angle may have a mechanism for specifying light beam positions on the object surface by arranging the device essentially at infinite distance.

The light source may be a left or right circularly polarized light panel, a left/right circularly polarized light switching panel, a panel of light-emitting diodes provided with a polarization film, or another type of panel, and may be suitably selected from among known light sources in the corresponding field of optical microscopes, confocal microscopes, fluorescent microscopes, polarization microscopes, and the like; and the light source is not particularly limited as long as the desired object can be achieved. Examples include white light sources and laser light sources that emit coherent light. Typical light sources include halogen lamps, xenon lamps, deuterium lamps, globar lamps, helium-neon (He—Ne) lamps, YAB lasers, light-emitting diodes (LED), semiconductor lasers, high pressure mercury lamps, metal halide lamps, high pressure sodium lamps, and other HID lamps (high-intensity discharge lamps). The light source may be a two-way light source that uses a white light source and a 408-nm violet laser diode or another short-wavelength laser light source, or a plurality of light sources that includes a reference origin of the incident plane azimuth angle and incident angle, in which the incident light beams are known. The light source may also be a single light source.

The light source may be configured to cause light from an above-noted luminous source to pass through a ¼ wave plate and a linear polarization plate, or may include a configuration that irradiates light from an above-noted luminous source that passes through an arrangement that surrounds the observed object and has a ¼ wavelength film layered onto a linear polarization film.

The light emitted from the light source may be inputted to an incident light optical system via an optical fiber as needed. In such an incident light optical system, it is possible to provide a light intensity stabilizer, a light density filter, or the like. The light in a substantially already-known polarization state may be obtained by passing the light emitted from the light source through a polarizer disposed in the incident light optical system. It is also possible to use a wave plate to convert linearly polarized light to circularly polarized light and/or convert circularly polarized light to linearly polarized light, or to rotate the polarized light axis of the linearly polarized light. The incident light beams, i.e., the illumination on the sample is uniformly irradiated from the periphery of the sample. The illumination device constituting the incident light optical system is capable of making polarized light essentially uniformly incident about the periphery of an object as the sample. The incident light optical system is controlled by a control system that operates in coordination with a computer and is capable of making light essentially uniformly incident about the periphery of an object as the sample. For example, in the case of a laser light source that produces a point light source constituting the reference origin of the incident angle and the azimuth angle of the incident plane and in which the incident light beams are already known, it is also possible to include an X-Y scanning optical system which is controlled by a control system that operates in coordination with a computer and which divides the observation field of view into a suitable number of pixels and carries out scanning.

The light beams reflected at the object surface can be detected as a polarized light image by a polarized light image detection device, e.g., a polarized light imaging camera, or the like, and can be introduced to a detection optical system provided with analyzers in order to detect the polarization state. The reflected light received by the detection optical system provided with analyzers passes through a spectroscope that may include a monochromator tuned to the optical band of the light source, and is thereafter fed to a photodetector and detected by a photoelement. A spectroscope is capable of analyzing the spectrum of the received light. In other words, the reception of light can be detected while the detection wavelength is varied. Optical fiber can be used for directing light to a predetermined apparatus. An advantage is obtained in that the movable components of a device and/or a movable device can be configured to move freely and independently of each other by using optical fiber.

The light can be converted to an electric signal using an optical sensor provided with a photoelement or the like. Examples of the optical sensor include a photodiode, diode array, charge-coupled device (CCD) image sensor, and CMOS image sensor, and may be combined with a photomultiplier (PMT).

The detection optical system in the incident light optical system and the polarized light image detection device in the above-noted illumination device may be provided as needed with wave plates; compensators; photoelastic modulators or other modulators; mirrors, slits, filters, and lenses (e.g., a condenser lens, or the like) for directing light beams; transparent plates; polychromators; and the like. The analyzers may be configured using polarizers. A monochromator may also be disposed in the incident light optical system. The polarizers of the incident light optical system and/or the analyzers of the detection optical system may be movable by drive units. The analyzers may be arranged so as to be capable of being rotated by a drive device under the control of a later-described computer system so as to analyze the polarization state. The wave plates may also be movable by drive units. It is also possible to provide drive units that include a rotation mechanism in the stage for the observed sample; and the entire incident light optical system and/or the entire detection optical system can be made movable by the drive units. The present invention can be advantageously used in a non-scanning scheme.

In the case of an analog image signal, the signal may be converted to digital by a converter as required. The signal is sent to a computer system and handed off to computation means to calculate the incident angle and the orientation of the incident plane. The gradient plane of the observed object from which the incident light is reflected and which is the surface of the object as the sample, i.e., the gradient of the tangent plane is determined and the shape of the object including the three-dimensional shape is reproduced. The reproduced shape can be made viewable and/or recognizable using a display device and/or an output device constituting the computer system. The computer system is provided with a data storage device and computation device, e.g., a hard disk drive and a CPU, and may also have a CD, MO, DVD, or another reading and/or writing device.

The drive units of the stage of the observed sample may be mutually independent and freely movable on the x-y-z axes under the electronic control of the stage controller of the computer system. An auto-stage may also be used advantageously. In the case that rotating polarizers are used, it is possible to set the continuous rotation of the polarizers by the electronic control of the computer system, and in the case that rotating analyzers are used, it is possible to set the continuous rotation of the analyzers by the electronic control of the computer system. In the case that wave plates are provided and are to be moved, such may be carried out by electronic control of the computer system, and there are cases in which such is preferred. The drive units are moved by electronically controlled stepping motors, and the operating data together with the position information, and the like may be collected by the computer system. Similarly, the control devices of the computer system may operate a monochromator to establish the synchronizing wavelength, and may operate a light source to control light flux or the like. The recording of position information, information about the polarization state including the wavelength and information about the shape of elliptically polarized light, information about the measurement position, and other collected data may be controlled and fed to a computation device (e.g., CPU) of the computer system as required.

The present computer system is provided with a predetermined data processing program, uses the collected data in accordance with an arbitrary suitable program, and reconstitutes measured images. Examples of the processing program include those having a function for comparing and calibrating measurement data under the incident light in a polarized state and measurement data under incident light in an unpolarized state; and those for carrying out a function for collecting data until sufficient data has been accumulated to characterize the data, a function for constructing a shape including a three-dimensional shape from the collected data, a function for displaying and/or providing output to a display device and/or an output device, a function for carrying out analysis using an optical model based on optical theory, and other functions. In the case of analysis using an optical model based on optical theory, it is also possible to include a process for comparing measured data and data obtained by optical modeling, and carrying out analysis using a regression analysis algorithm.

The case in which circularly polarized light is used as the polarization state of the incident light has been described to this point, but the illumination light may fundamentally be a known polarization state, and it is also possible to use a configuration that makes use of elliptically polarized light or linearly polarized light. Generally, it is possible to fabricate a linear polarizer for any wavelength region. It is also possible to used recently reported axisymmetric polarized light beams (“Jiku Taisho Henko Bimu” (Axisymmetric Polarized Light Beams) by Yuichi Kozawa and Shunichi Sato, Kogaku, Vol. 35, No. 12 (2006), pp. 9-18 (Non-patent Document 5) as the illumination light. The axisymmetric polarized light beams can be readily obtained using photonic crystal optical elements. For example, a concentric or radial polarized light beam can be obtained by using a concentric photonic crystal polarizer in place of an output mirror of a laser generator. It is possible to set the reflectivity to a level appropriate for laser oscillation because the reflectivity can be readily adjusted in the case that a photonic crystal is used, and high-durability performance can be obtained because the constituent materials are inorganic. In relation to an axisymmetric photonic crystal, it is also possible to include a laser resonator half mirror for oscillating a concentric or radial polarized light beam; a polarizer having a concentric or radial transmission axis; a ½ wave plate integrated element for converting linearly polarized light into concentrically/radially polarized light; or the like.

The technique of the present invention may include a method and device for simultaneously determining the optical properties using ellipsometry. For example, an illumination device in the optical device for shape and gradient detection and/or measurement of the present invention includes a spatially specified incident light beam as a measurement reference origin, and is capable of specifying the optical properties of the reflection surface from the observed values of a polarized light ellipse at a reflection point specified by the polarized light image detection device. With such a configuration, it is possible to vary the color during illumination, form holes, and ellipsometrically measure a single point in a polarized light image.

The gradient ellipsometry of the present invention includes measurement and application using unpolarized illumination used in robotics applications. The fundamentals of gradient ellipsometry of the present invention can be described to include a partial polarization state using a Stokes parameter and a Mueller matrix. When reflection at the sample surface includes a scattering process, the reflected light becomes partially polarized light. Changes in the polarization state due to reflection are generalized, and a Stokes parameter and a Mueller matrix capable of expressing unpolarized and partially polarized light are used.

The formula that corresponds to

$$[ \begin{matrix}{E}_{ox} \\ {E}_{oy}\end{matrix} ] = {T}_{{θ}_{1}} {R}_{{φ}_{1}} {T}_{- {θ}_{1}} [ \begin{matrix}- i \\ 1\end{matrix} ] \qquad [ Formula 25 ]$$

is

$$[ \begin{matrix}{S}_{0} \\ {S}_{1} \\ {S}_{2} \\ {S}_{3}\end{matrix} ] = {T}_{{θ}_{1}} {R}_{{φ}_{1}} {T}_{- {θ}_{1}} [ \begin{matrix}1 \\ 0 \\ 0 \\ 1\end{matrix} ] \qquad [ Formula 26 ]$$

for the case of right circularly polarized light using a normalized Stokes parameter in which intensity is normalized to 1 in Mueller calculation.

Here, the rotator matrix of the angle θ that accompanies the rotation of the coordinate system is

$${T}_{θ} = [ \begin{matrix}1 & 0 & 0 & 0 \\ 0 & cos 2 θ & - sin 2 θ & 0 \\ 0 & sin 2 θ & cos 2 θ & 0 \\ 0 & 0 & 0 & 1\end{matrix} ] \qquad [ Formula 27 ]$$

and the following is the Mueller matrix that expresses reflection of polarized light at the incident angle φ₁ at the perpendicular sample surface to which the incident plane is horizontal.

$${R}_{{φ}_{1}} = [ \begin{matrix}1 & - cos 2 {ψ}_{1} & 0 & 0 \\ - cos 2 {ψ}_{1} & 1 & 0 & 0 \\ 0 & 0 & sin 2 {ψ}_{1} cos {Δ}_{1} & sin 2 {ψ}_{1} sin {Δ}_{1} \\ 0 & 0 & - sin 2 {ψ}_{1} sin {Δ}_{1} & sin 2 {ψ}_{1} cos {Δ}_{1}\end{matrix} ] \qquad [ Formula 28 ]$$

These matrices are substituted into

$$[ \begin{matrix}{S}_{0} \\ {S}_{1} \\ {S}_{2} \\ {S}_{3}\end{matrix} ] = {T}_{{θ}_{1}} {R}_{{φ}_{1}} {T}_{- {θ}_{1}} [ \begin{matrix}1 \\ 0 \\ 0 \\ 1\end{matrix} ] \qquad [ Formula 29 ]$$

to produce

$$\begin{matrix}[ \begin{matrix}{S}_{0} \\ {S}_{1} \\ {S}_{2} \\ {S}_{3}\end{matrix} ] = {T}_{{θ}_{1}} [ \begin{matrix}1 \\ - cos 2 {ψ}_{1} \\ sin 2 {ψ}_{1} sin {Δ}_{1} \\ sin 2 {ψ}_{1} cos {Δ}_{1}\end{matrix} ] \\ = [ \begin{matrix}1 \\ - cos 2 {θ}_{1} cos 2 {ψ}_{1} - sin 2 {θ}_{1} sin 2 {ψ}_{1} sin {Δ}_{1} \\ - sin 2 {θ}_{1} cos 2 {ψ}_{1} + cos 2 {θ}_{1} sin 2 {ψ}_{1} sin {Δ}_{1} \\ sin 2 {ψ}_{1} cos {Δ}_{1}\end{matrix} ]\end{matrix} \qquad [ Formula 30 ]$$

In other words, variation due to the slope θ₁ of the incident plane is not accompanied by variation in the ellipticity angle because only the S₁, S₂ coordinates rotate about S₃ as the rotating axis on a Poincaré sphere.

In the case that unpolarized light is incident, the unpolarized light is substituted in place of the incident Stokes parameter as in the following

$$[ \begin{matrix}{S}_{0} \\ {S}_{1} \\ {S}_{2} \\ {S}_{3}\end{matrix} ] = {T}_{{θ}_{1}} {R}_{{φ}_{1}} {T}_{- {θ}_{1}} [ \begin{matrix}1 \\ 0 \\ 0 \\ 1\end{matrix} ] = [ \begin{matrix}1 \\ - cos 2 {θ}_{1} cos 2 {ψ}_{1} \\ - sin 2 {θ}_{1} cos 2 {ψ}_{1} \\ 0\end{matrix} ] . \qquad [ Formula 31 ]$$

The information of the phase angle of reflection is lost with the incidence of unpolarized light, but Ψ₁ indicating variation in the intensity reflectance of the p- and s-components can be measured. Therefore, the incident angle φ₁ can be determined from the incident angle dependency of Ψ₁ in a transparent sample.

Incident angle dependency of the degree of polarization is used in shape measurement in the robotics field. The degree of polarization V of partially polarized light is defined and calculated using

$$V = \frac{\sqrt{{S}_{1}^{2} + {S}_{2}^{2} + {S}_{3}^{2}}}{{S}_{0}} = \vert cos 2 {ψ}_{1} \vert \qquad [ Formula 32 ]$$

as the ratio in relation to the partially polarized light of the perfectly polarized light component.

The degree of polarization in terms of real numbers is a measurement method that is naturally limited in application in comparison with the method of the present invention in which the information of two variables of a polarized light ellipse are measured.

The reason that the applicable range of shape measurement under unpolarized illumination is limited to transparent bodies is that the incident angle dependency of the absolute value of Ψ₁ is used and this is a limited measurement condition. Since the function is an odd function having a minimum value, there are two incident angles that give the same degree of polarization, and algorithms must be written to determine the true value (D. Miyazaki, M. Saito, Y. Sato, K. Ikeuchi, “Determining surface orientations of transparent objects based on polarization degrees in visible and infrared wavelengths,” J. Opt. Soc. Am. A, 19(4), pp. 687-694, 2002). In the present invention, such algorithms are unnecessary because a signed Ψ₁ can be determined.

In the case that absorption is high, the incident angle dependency of Ψ₁ is generally low, as shown in FIGS. 6 and 7. Therefore, shape measurement using unpolarized illumination of robotics applications cannot be used with such sample objects. Light having a wavelength for which an object is transparent is used in the case of special applications in which the method of the present invention cannot be used and the use of unpolarized illumination is desired. Light having a wavelength for which an object is transparent is light that is transmissive in the infrared region in the case that the extinction coefficient k of the imaginary part of the complex refractive index n-ik is small, e.g., when the target object is Si, or the like. Measurement using unpolarized illumination is the case of measurement using, e.g., sunlight or the like in which the light source cannot be polarized, and in accordance with the theoretical system disclosed in the present invention, it is apparent that measurement sensitivity can be improved by setting the wavelength to be detected to a wavelength for which an object is transparent. As described above, the information of Δ is lost in the use of unpolarized illumination, so incident angle dependency is measured using only the information of Ψ. Measurement is possible using only linear analyzers, but retarders are generally required to confirm that the illumination light is unpolarized light.

All substances are transparent in the soft X-ray region. Polarizers and retarders have been developed for the soft X-ray region, and detection and/or measurement of the present invention can be implemented using a radiation light source, a laser-generated plasma soft X-ray light source, or the like. In such cases, the reflectivity of the substance is very low and it is therefore preferred that a region near grazing incidence be mainly used.

In another aspect in the present invention, there is provided a method for extracting object information, i.e., a method for optical shape and gradient detection and/or measurement to detect and/or measure the shape and gradient of a surface of an observed object using the reflectance optical characteristics of the surface of the object. The method is a for optical shape and gradient detection and/or measurement to detect and/or measure the shape and gradient of a surface of an observed object using the reflectance optical characteristics of the surface of the object, and is characterized in causing light surrounding the periphery of the object to be uniformly incident, the light being from an illumination device and in a substantially already-known perfectly polarized state; detecting with a polarized light image detection device the polarization state of a group of light beams specularly reflected by the object surface and emitted at a specific azimuth angle; measuring a gradient angle with respect to the radiated light beam of the reflection surface by detecting the incident angle and the orientation of the incident plane from the ellipticity and the azimuth angle of the polarized light ellipse for the refection surface of the object that forms an incident point for each reflected and radiated light beam (e.g., including detecting the orientation of the incident plane from the azimuth angle of the polarized light ellipse, and detecting the incident angle from the ellipticity of the polarized light ellipse); and extracting object information using a technique that includes that fact that the measured gradient angle smoothly varies on the object surface. Each specific technique is the same as described above. The method may be used for detecting and identifying specific changes in the surface gradient angle caused by various pathological abnormalities, including malignant tumors, the object of detection and/or measurement being a human body or a portion of a human body including a breast. The method includes imparting predetermined deformation changes in the orientation or the like of the observed object, which includes a patient or the like, and detecting and/or measuring changes in the gradient angle before and after deformation. The method may be used for detecting and/or measuring changes in the optical characteristics of a reflection surface using illumination light as white light and in which the observed object surface including skin is essentially used as the reflection surface in which consideration is given to the fact that depth of penetration from the surface changes in accompaniment with the wavelength.

(Range of application)

<Direct Application of Surface Gradient Angle Data: Normalized Shape Data>

Application of the present invention is not limited to the applications described above. The surface gradient angle data that can be measured by gradient ellipsometry is not limited to shape measurement, it is also possible to make application to database construction or the like using object shape sampling, statistical processing, and the like. In the method of the present invention, e.g., gradient ellipsometry, the surface gradient is also recorded for measurements in which the magnification has been varied and therefore only information related to the shape is extracted.

Therefore, even if the size varies due to individual differences, the shape can be scaled.

<Other Wavelength Applications: Wavelength not Limited: White Circularly Polarized Light can be Used>

The gradient ellipsometry method of the present invention can be used for all electromagnetic waves, is particularly different from other methods that make use of interference, and is capable of using white light unchanged. Therefore, the surface region characteristics in the range of the penetration depth can be evaluated using measurement results of other wavelengths when shapes are measured using UV light or other light having wavelength in which the absorption coefficient is high and the penetration depth of light is poor. For example, the optical characteristics of the surface can be more finely detected because wavelength dispersion of the complex refractive index can be expressed as a dispersion formula having about three variables.

<Gradient Angle Measurements are not Affected by External Disturbances: the Measurement Environment Irrelevant>

Gradient angle measurements of gradient ellipsometry of the present invention are sensitive to the incident angle, are not sensitive to horizontal and vertical shift components caused by vibrations or the like from external disturbances, and are therefore suitable for precision measurements in ordinary environments.

<Suitable for Precision Gradient Measurements in Ordinary Environments; Capable of Extracting Local Deformations Using a First-Order Differential Amount of the Gradient Change>

For example, the gradient can be read directly by using [the method] in the observation of the human body or a breast and other body parts. The tissue and dynamic characteristics of a malignant tumor or the like differ from normal cells under the skin, and are not uniform. Therefore, although a diagnosis has been made by palpation by a doctor, slight concavities, convexities, and other local deformations can be readily detected by differential amounts in the data by applying the gradient ellipsometry of the present invention. In particular, it can be expected that the modality of the deformations will be clearly distinguishable from homogeneous normal cell portions by observing mammary deformations by changes or the like in the orientation of the patient. It is also possible to produce predetermined deformations by applying constant pressure to the skin using an airflow or the like, or scanning the mammary surface as required. Another method that holds promise is to discover deformation abnormalities in deeper locations by imparting even greater pressure changes using a liquid flow and making observations using a configuration in which the breast is enveloped in a fluid matched to the body temperature. It is also may be possible to select observation wavelengths to detect the characteristics of vascular tissue which has developed in the area of a malignant tumor. In optical observation of living tissue in humans and the like, light scattering measurement methods that actively make use of scattering phenomena and the coherence of illumination light are being developed. It may be possible to make new progress in research by incorporating the fact that “the optical characteristics of perfectly polarized light component of the reflected portion of polarized light has characteristic incident angle dependency” as disclosed in the present specification.

<Capable of Readily Recording Temporal Changes; Suitable for Dynamic Observation>

The gradient ellipsometry of the present invention is capable of detecting very small local deformations in an image by recording temporal changes because the direct reading of the gradient is carried out simultaneous to image acquisition. This function can develop into applications for suppressing precursory phenomena or the like by researching the dynamics of, e.g., cell division, apoptosis, and the like.

<Observation Using Sunlight or Radio Waves: Observation of Specularly Reflected Light by Parallel Illumination: Reverse Optical Path of FIG. 1>

Examples of other similar applications include detection of deformations in the surface of the earth by satellite imaging, and detection of deformations in the surface of the ocean. In this case, the illumination light is sunlight at infinite distance, and although observation is carried out with the reverse direction of the light beams of FIG. 1, the fundamentals of reflection are the same. It is also possible to make application to measurement by radio waves.

<Interface Observation of Solids, Liquids, Gases, and Combinations Thereof>

Additionally, the method is also useful in applications for observing the shape and dynamics of liquid and droplet surfaces, in which the application of other methods is difficult. This includes observation of steps and facets of a crystal growing in a melt. The measurement target can be generalized to the interface between a liquid in a gas, or a gas in a liquid. Similarly, application can be expanded to include the interface between a solid and a liquid, a liquid in another liquid having a different density, a gas in a gas, a solid in a solid, and all other substance interfaces that can produce reflection. These are situations that are difficult or impossible to measure using conventional interferometric methods and the like.

<Observation in Extreme Environments>

The gradient ellipsometry of the present invention is furthermore a remote sensing method, and is therefore thought to be useful in measurements of dynamics and measurements of shapes of regularly and irregularly shaped objects in high temperature, high pressure, and in other environments in which ordinary methods are difficult to use.

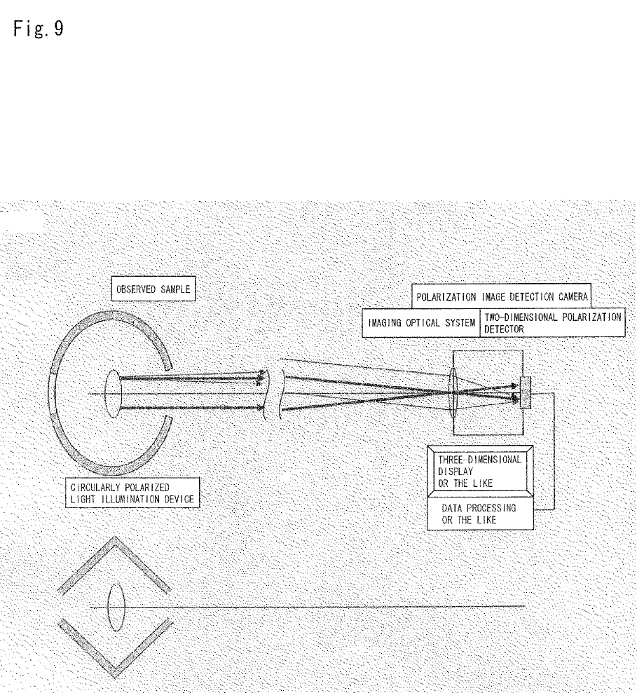

An example of the first embodiment of the present invention is a shape-measuring telescope such as that shown in FIG. 9. In relation to the shape-measuring telescope, the first example may have right or left circularly polarized light as the illumination light. The second example is the case in which left and right circularly polarized light are switched.

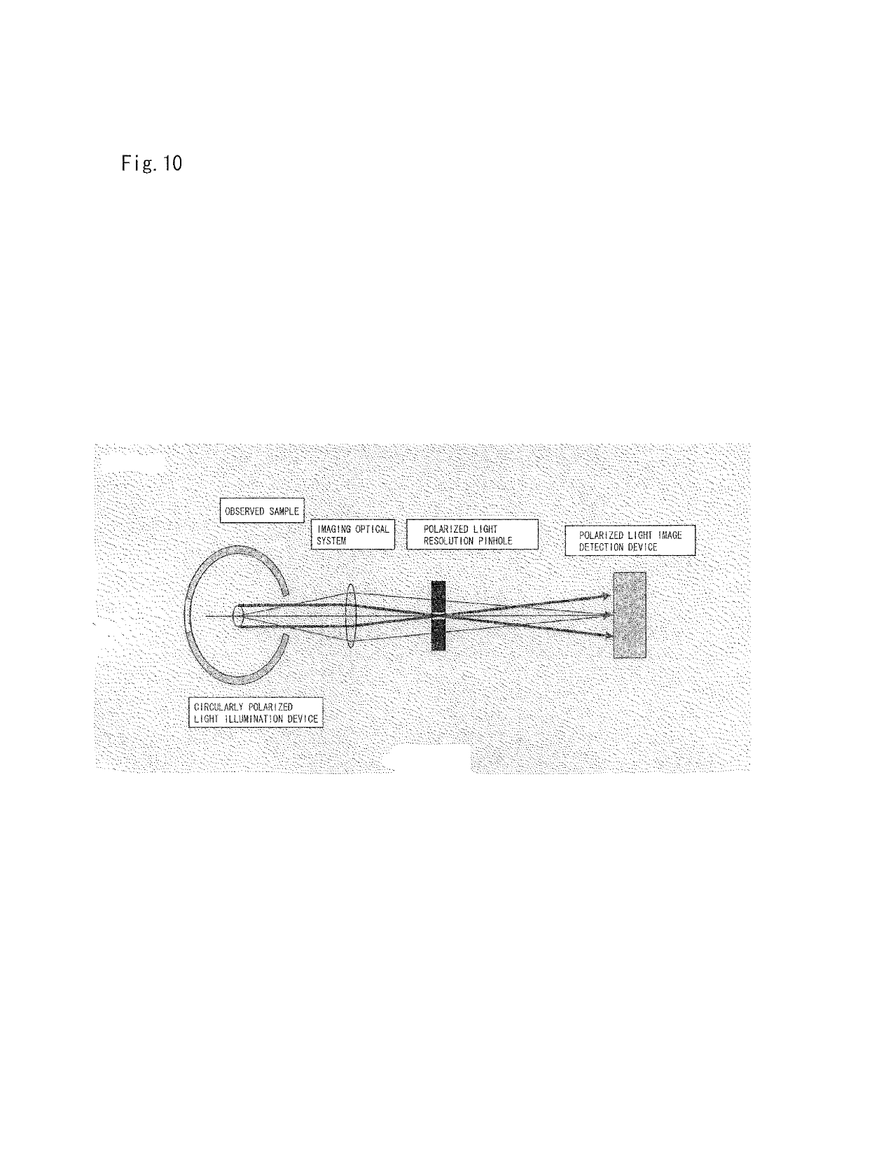

An example of the second embodiment of the present invention is a shape-measuring microscope such as that shown in FIG. 10. In relation to the shape-measuring microscope, the first example may have right or left circularly polarized light as the illumination light. The second example is the case in which left and right circularly polarized light are switched.

As described above, in the gradient ellipsometry of the present invention, a specific configuration brought together in view of two reflection polarization characteristics, which are characteristics shared by all substances: I. the complex amplitude reflectance ratio ρ is −1 when the incident angle φ is 0°, and is 1 when φ=90°, and II. ρ varies monotonically from −1 to 1 on a complex plane when the incident angle φ varies from 0° to 90° and invariably passes through an imaginary axis (Δ=±90°) at a midway point in which the real number part of ρ is 0. In other words, the periphery of the sample is uniformly illuminated with circularly polarized light, the polarization state of the specularly reflected light is observed from a spatially fixed direction, and the incident angle (reflection angle) and the slope of the incident plane at the reflection point are measured from the shape of the observed elliptically polarized light thus reflected for an arbitrary reflection point on the cross-sectional coordinates of the sample. Provided is a simple general purpose method for detecting shapes and gradients and/or measuring and analyzing shapes in which the observed data of the gradient can be directly used. The method may furthermore be used in three-dimensional shape measurement applications or the like for reconstructing the shape of a sample by smoothly connecting the reflection surfaces of the measured reflection points, between the measurement points in sequential fashion within the sample cross section; and may be used for improving the precision of known methods by using perfectly polarized illumination in robotics applications being developed solely for transparent bodies using unpolarized illumination in the three-dimensional shape and measurement applications.

Modes of the present invention are described below using specific examples of the embodiments of the present invention, and the specific modes are merely provided as reference for describing the present invention. The examples are used for describing specific details modes of the present invention and do no limit the scope of the invention disclosed in the present application and do not represent a limitation. In the present invention, it should be understood that various embodiments based on the concepts of the present specification are possible. All examples are examples that have been implemented or can be implemented using standard techniques, and such is common knowledge to those skilled in the art.

### Example 1

### Reduction Optical System Device

An example of the first embodiment of the present invention include a shape-measuring telescope, which is a reduction optical system device. FIG. 9 shows the configuration of a shape-measuring telescope in the most simple configuration. The present configuration can be applied to a reduction optical system device and may also naturally be applied to camera or the like.

The illumination device in the shape and gradient measurement optical device of the present invention is shown as a circularly polarized light illumination device in FIG. 9. The circularly polarized light illumination device can be implemented by surrounding the periphery of the sample with a circularly polarized light panel. Technical elements of the circularly polarized light panel may be composed of elements similar to a liquid crystal panel, and may include a light-emitting diode or another light source, a diffusion plate, a linear polarization film, and a retarder film, and includes configurations in which the circularly polarized light panel has uniform brightness. In practical terms, the configuration may be one in which the retarder film is applied in a predetermined orientation to the surface of a liquid crystal panel acting as the linear circularly polarized illumination to form a circularly polarized light panel. When the configuration is an illumination panel having uniform in-plane brightness using a diffusion plate, the brightness is established without relationship to the observation direction by the fundamentals of photometry, and shapes cannot be distinguished. Therefore, the illumination device can be, e.g., a box shape composed of panels. Essentially, the sample may then be placed in the box in which each surface is a liquid crystal panel.

The polarized light image detection device in the shape and gradient measurement optical device of the present invention is shown on the right side of FIG. 9. A polarized light imaging camera (Photonic Lattice, Inc. (Aoba-ku, Sendai-shi, Miyagi-ken)) provided with a two-dimensional polarized light detector can be advantageously used for polarized light image detection in FIG. 9. In this manner, polarized light image detection can be advantageously carried out by using a photonic crystal element, or by using a photonic crystal element and a charge coupled device (CCD). In the camera, the image signal can be sent to a personal computer via a USB cable and processed using suitable software. Examples of the photonic crystal element include a polarizer array (patterned polarizers) and a λ/4 wave plate array (patterned wave plates). The polarized light imaging camera may be configured using any components selected from the group comprising a collimator, a prism, a wave plate array, a polarizer array, and a CCD. A preferred configuration is one that incorporates image information in a spatially parallel fashion. In a typical configuration, the image is processed for each pixel and the image data can be machine recognized.

An example of the polarizer array is a chip in which a about a million or another predetermined number of polarizers having the same size as the pixels is closely packed into an array. For example, the polarizer array may have substantially square-shaped polarizers with slightly different transmission axis orientations closely packed into an array, and the brightness of four pixels in close proximity in the polarizer array is computed, whereby the major axis of the polarized light, the average brightness, and the strength of the polarized light component can be obtained instantaneously. Also, the polarizer array may have a configuration in which longitudinal polarizers with slightly different transmission axis orientations are arranged in the horizontal direction, or the wave plate array may have a configuration in which oblong wave plates are conversely arranged in the longitudinal direction.

Photonic crystals have a structure in which materials having different refractive indexes are arranged in a periodic fashion, and the periodic structure is multidimensional such as two dimensions or three dimensions. The period of the structure is ordinarily designed to be about half the wavelength of the light to be used; for example, if it is used in the visible light region, the photonic crystals are designed and fabricated so that the period is about 300 nm. Although the periodic structure of a photonic crystal is referred to as a “crystal,” the periodic structure of this photonic crystal is about several 100 nm, and forms a multidimensional structure in which the “photonic band” of the waveband through which light passes and the “photonic band gap” of the waveband that cuts off the passage of light are arrayed and/or layered; that is, a multilayered structure in which two dielectric bodies having a high refractive index and a low refractive index are self-shaped while maintaining fixed concavities and convexities. A typical photonic crystal is manufactured by a technique for combining bias switching with sputter-layering on a patterned concavo-convex substrate to form a regular multidimensional layer and pattern such as a three-dimensional concavo-convex pattern; for example, an auto-cloning method. The film formation material may be any of various materials, examples of which include Si, SiO₂, TiO₂, Ta₂O₅, Nb₂O₅, and rare earth oxides. Photonic crystal elements have a function for controlling the transmission, reflection, and refraction characteristics of light.

Two-dimensional distribution data of Stokes parameters are outputted from a two-dimensional polarization detector of the camera through a data processing unit, and can thereafter be processed as necessary by providing, for example, a data processing system, a display device, and a data storage device.

FIG. 9 schematically shows the state of light rays emitted from the upper side of an object forming an image in the lower portion of the camera on the right (the displacement angle from the optical axis has been accentuated). A reducing optical system such as a telescope generally has such a configuration, by which the polarization state may be viewed evenly because the aperture angle of the light flux that diverges from a single point on an object and is used for image formation is sufficiently small.

### Example 2

### Magnifying Optical System Device

An example of the second embodiment of the present invention is a shape-measuring microscope, which is a magnifying optical system device. FIG. 10 shows the configuration of a shape-measuring microscope in the simplest configuration. The present configuration can be applied to a magnifying optical system device, and may also be used in various devices without particular limitation as long as the object can be achieved.

In a magnifying optical system for microscopic measurements and the like, the aperture angle in an imaging system for light flux diverging from a single point on a sample increases as the numerical aperture (NA) of the optical system is increased. Changes in the polarization state in the light flux are thus significantly large enough that the configuration is generally fashioned as shown in FIG. 10.

When forming an image in the polarized light detection device in a magnifying optical system, the NA of the optical system must be increased in order to increase image resolution. However, since increasing the NA increases the angle at which a component reflected at a single point of a sample is taken in, this also increases the angle of incidence and reduces resolution in relation to the polarization state, which is a function of the angle of incidence. A polarized resolution pinhole is inserted and used in the case that a higher polarized resolution is desired. Thus, the light beam components indicated by bold lines can be brought out as shown in FIG. 10. Measurements are actually carried out by superimposing an image, which has a polarized resolution obtained by inserting a pinhole, onto an image having high spatial resolution obtained with the polarized resolution pinhole removed.

The function of the present pinhole can also be achieved by using a photonic crystal wave plate array combining two quarter-wave plates with mutually orthogonal anisotropic axes. For example, two types of wave plates having different anisotropic axes and that have been joined essentially without a joint boundary, i.e., a vertical polarization slit, may be used (Photonic Lattice, Inc. (Aoba-ku, Sendai-shi, Miyagi-ken)).

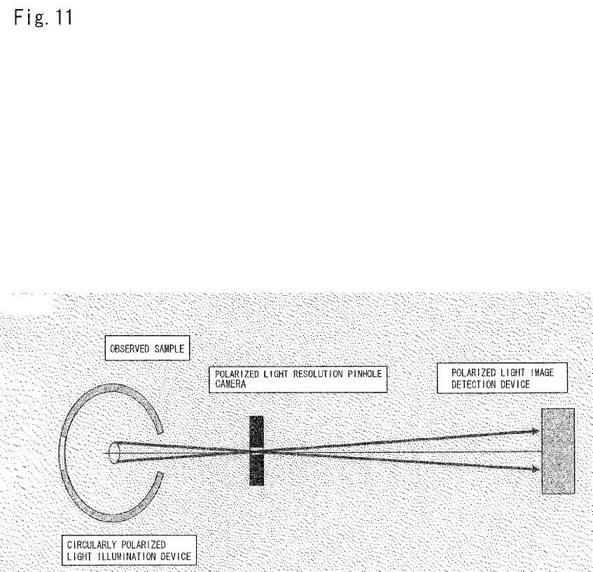

In applications in which spatial resolution of image formation is not important, the image formation optical system can be omitted. This gives an even more simplified configuration. Magnification or reduction can be freely selected by the positional relationship of the pinhole and the detector. See FIG. 11.

The present invention may be designed to enable acquiring a plurality of polarized light images by the configuration described above, and preferably is designed to enable increasing precision.

Components such as the illumination device, the polarized light image detection device, and the data processing system may be configured in the same manner as example 1.

### Example 3

### Other Shape-Measuring Optical Devices

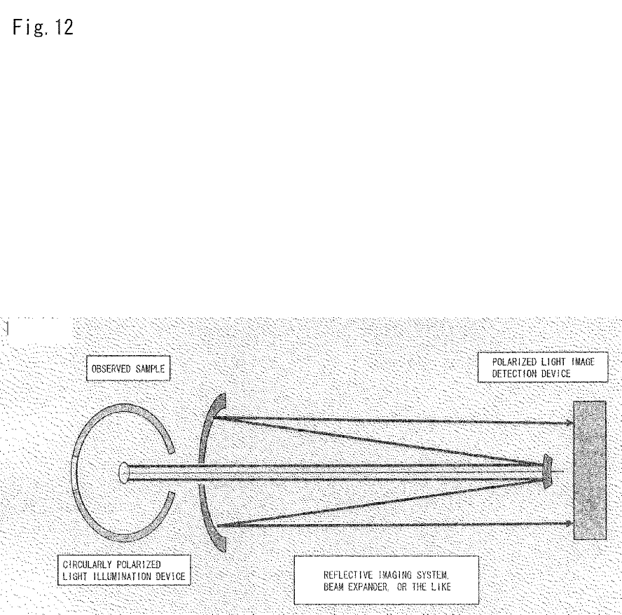

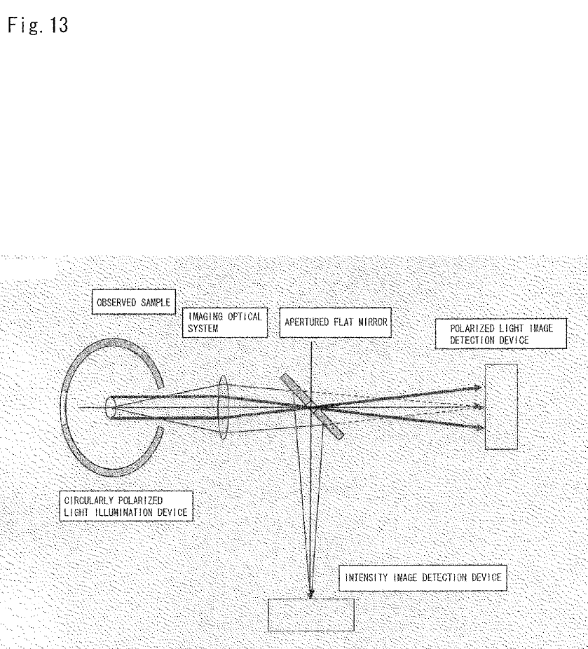

The optical device for shape and gradient measurement of the present invention may be, for example, a device such as shown in the schematic diagrams of FIG. 12 or 13. It should be understood that the depicted devices can be configured using a combination of known techniques, and that many alterations and modifications can be made. The optical system and the collimator optical system may be configured using lenses, or the optical system may be configured using mirrors in an optical system that uses white light or multi-wavelength light. In the case of a configuration such as shown in FIG. 12, variation in the phase and amplitude at the mirror surface may be measured and corrected in advance as required. A folding reflector may have an aperture in the center. In a typical configuration, the optical system between the polarized light illumination device and the polarized light image detection device may include a reflection imaging system, a beam expander, or the like.

Next, the configuration shown in FIG. 13, for example, may be used for simultaneously observing an image of high-spatial-resolution intensity and a high-resolution polarized light image without pinhole switching. In a typical configuration, the optical system between the polarized light illumination device and the polarized light image detection device may include an image formation optical system, a flat mirror with a pinhole, or the like, and may further include an intensity image detection device or the like.

### Example 4

### Mammography Device

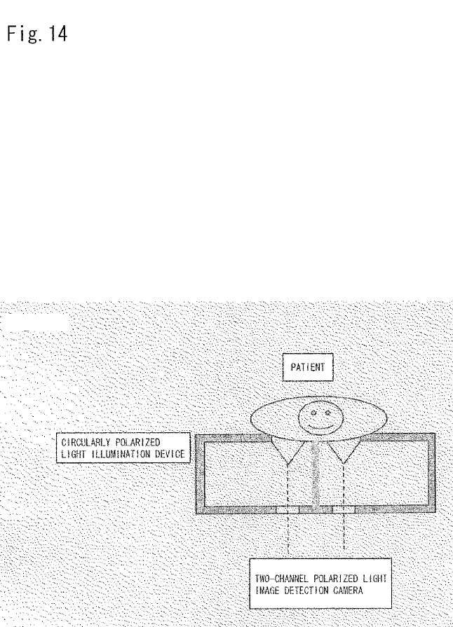

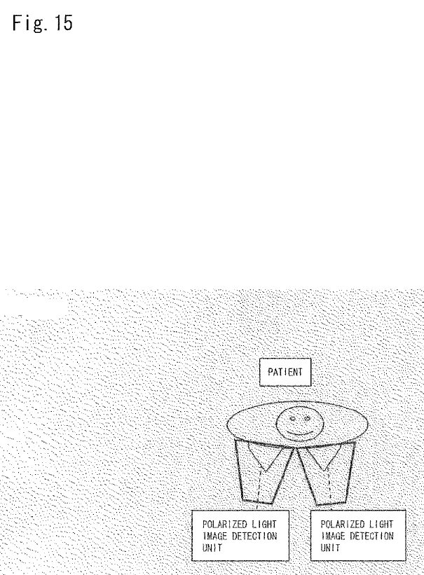

The optical device for shape and gradient detection and/or measurement of the present invention may be, for example, a device such as shown in the schematic diagrams of FIG. 14 or 15. The optical device for shape and gradient detection and/or measurement of the present invention may be configured as a medical diagnostic device that includes mammography. Such a medical diagnostic device can detect and identify specific changes in a surface gradient angle caused by various pathological abnormalities, including malignant tumors, for a human body or a portion of a human body, including a breast, as the object of detection and/or measurement.

The optical device for shape and gradient detection and/or measurement of the present invention may be characterized in that a predetermined deformation is imparted by a process that includes changes in the orientation of an observed object, including a patient, and detecting and/or measuring changes in the gradient angle before and after deformation. The predetermined deformation may also be a change in the pressure exerted on the skin by an airflow, a fluid flow, or the like. The optical device for shape and gradient measurement of the present invention includes a configuration that is characterized by detecting and/or measuring changes in the optical characteristics of a reflection surface using the illumination light as white light and the surface of an observed object, including skin, as a substantially reflection surface, considering that the depth of penetration from such a surface changes with the wavelength.

A configuration such as shown in FIG. 14 may be used in mammography applications or the like, where a scheme may be considered in which an illumination device is mounted on a bed configuration having two apertures and the patient lies face down in a predetermined position on the bed. In this case, in a configuration in which the detection area is filled with a liquid set to body temperature and the breasts are enveloped in the liquid, a considerable pressure change can be applied using a liquid flow and observed in the expectation of finding deformation abnormalities in deeper locations. A configuration such as shown in FIG. 15 may be used when carrying out an examination in a standing position is desired, such as for a mass screening, in which case, a configuration may be used in which the chest area is pressed against a cylindrical examination unit. In this case, a constant pressure can be applied to the skin using an airflow or the like, and the surface of the breast can be scanned as necessary.

Components such as the illumination device, the polarized light image detection device, and the data processing system may be configured in the same manner as example 1. An auxiliary equipment for observation with the unaided eye, which allows a doctor to readily detect very small concavities or the like, may be used in the present mammography application or the like. This detection does not necessarily require quantitative measurements, provided that a function is provided for converting concavities and convexities in the affected areas into contrasting light and dark areas.

For example, in the simplest configuration, the present invention can be used by illuminating the affected areas with right circularly polarized light, and having the doctor wear elliptical polarizers in the form of glasses configured by combining quarter-wave plates and linear polarizers that can be rotated through different orientations, to observe very small concavities and convexities in an affected area with the unaided eye as variations in intensity. The glasses may be glasses used in ophthalmological examinations in which two lenses can be interchangeably mounted, or quarter-wave plates and linear polarizer plates may be mounted instead of two lenses. The doctor may observe an affected area using, for example, a left circular polarizer azimuth-angle configuration, then rotate the azimuth angle of the quarter-wave plates and the linear polarizers as necessary to change the elliptical shape of the elliptical polarizers so as to form a near extinction state, and use a state of high contrast or the like of shape changes to make detailed observations. This also includes a configuration in which the rotation of the azimuth angle of the quarter-wave plates and linear polarizers is automated to form an image using a video camera. In such a device, the image can be magnified on a screen for observation and other image processing. Circularly polarized illumination may be applied in various configurations so as to reduce psychological stress on the patient. For example, the cylinder of FIG. 15 may be a transparent circular polarizer cylinder formed by layering a quarter-wave film on a linear polarizer film, and which illuminates an affected area with ordinary external light, or the walls of the examination room may be configured with circular polarizer panels.

### Example 5

### Ellipsometric Shape-Measuring Optical Device

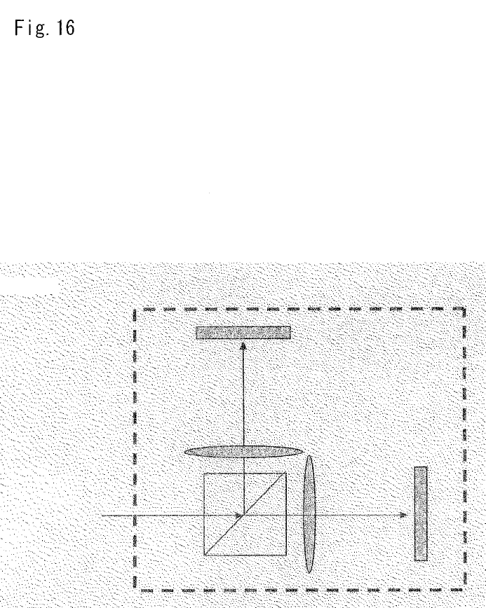

The shape-measuring optical device of the present invention may be, for example, a device such as shown in the configuration diagrams of FIGS. 16 to 18. In the present invention, conventional ellipsometric principles can be used for detecting the polarization state.

When classified by whether the polarization state to be detected does not require distinguishing the sign of the ellipticity angle, i.e., right polarization or left polarization, or requires that the measurement include the sign of the ellipticity angle, there are two configurations of polarization measurement:

(A) a configuration based on a “rotating analyzer method” for detecting the azimuth angle θ and the absolute value of the ellipticity angle of the polarization ellipse, and

(B) a configuration based on a “rotating retarder method” for detecting the azimuth angle and the ellipticity angle, including the sign (+ for right and − for left).

In the case of the “rotating analyzer method” of (A), detection is carried out by determining values S₁ and S₂, or the ratio thereof, on a plane perpendicular to an S₃ axis in which the ellipticity angle is fixed in terms of a Poincaré sphere. Since the S₃ information or the information about the rotational direction of the ellipse is not required, a phase shifter such as a quarter-wave plate is not required.

In (B), the detection optical system includes a phase shift such as a quarter-wave plate in order to identify the rotational direction of the ellipse, including S₃, and detection techniques related to a “rotating retarder method” that can determine Stokes parameters are used.

The conventional ellipsometry techniques already discussed can be used in these measurements.

In applications that do not require high-speed reading, on the other hand, the configuration preferably does not include a mechanical drive unit, even in the case of detection by the “rotating analyzer method” of (A). A configuration can be used that modulates the polarization state using polarization modulation elements that make use of polarization modulation effects such as the Faraday effect, the Kerr effect, or the Pockels effect, and determines the phase angle of the cos θ signal using a lock-in detection scheme. From the standpoint of high speed, however, an even more preferred scheme is to spatially divide reflected light into a plurality of beams, and allot a plurality of analyzers capable of detecting a specific polarization state to simultaneously detect polarization states in parallel.

For example, linear analyzers having a different azimuth angle θ for each channel of a detector having a plurality of channels may be allotted to simultaneously detect rotating retarder signals and output the phase angle (the azimuth angle of the major axis of the ellipse) of a cos θ signal obtained by signal processing as a multi-bit signal having the required number of significant digits. The number of channels is a minimum of three. Measurement precision may be improved by increasing the number of channels. The detection azimuth angles handled by the channels are arranged so as to be separated as much as possible on the S2-S3 plane of a Poincaré sphere, and at mutually equidistant intervals.

The orthogonal linearly polarized light image detection unit (orthogonal unit) of FIG. 16 is used in one channel of a two-dimensional polarization detector. A polarized light beam emitted from an object is divided by a polarizing beam splitter into a p-component that proceeds directly forward and an s-polarized component that is reflected, and the two components are formed by an image lens into an object image on a two-dimensional detector and drawn as orthogonal polarized light image outputs. The unit surrounded by broken lines is hereinbelow referred to as an “orthogonal unit.” The two polarized light images outputted from the crossed unit are positioned at symmetric points on a Poincaré sphere.

Therefore, when a polarization detection channel has been arranged at an azimuth angle of 0° of the horizontal linearly polarized light, the detected polarization state is composed of an image produced by the horizontal linearly polarized light (front channel) and a vertical linearly polarized image (back channel).

When the rotating analyzer scheme of (A) is configured with three channels, for example, the detection azimuth angles are based on an equidistant arrangement of 60° on a Poincaré sphere; for example, 0° (back 180°), 60° (back 240°), and 120° (back 300°). The azimuth angles in real space are one-half of these angles: 0° (back 90°), 30° (back 120°), and 60° (back 150°). In real space, 180° azimuth angle rotation is completely equivalent in terms of polarization state and cannot be differentiated. Therefore, azimuth angle rotation can be set in equidistant intervals of 120° at 0°, 120°, and 240° in a real space arrangement. Another potential configuration is to use linear analyzers at these azimuth angles, in which case, the signal contrast of each channel can be optimized by arranging predetermined elliptical analyzers in each channel using phase shifters such as quarter-wave plates. The number of channels is determined by the measurement precision required; for example, fifteen channels can obtain 30 units of image information and a measurement precision of 1/1000°.

In the Stokes parameter detection scheme of (B), in principle, a signal varying the phase of the polarization state can be obtained using a phase shifter such as a quarter-wave plate. In this case as well, the configuration preferably does not include a mechanical drive unit for the rotating retarder detection in order to achieve high-speed reading. In principle, the configuration of the detection system is the same as in (A), with specific analyzers arranged in a plurality of channels. The analyzers in this case are elliptical polarized light analyzers, including linear polarizers and circular polarizers, orthogonal to a specific elliptical polarization state. The optimal configuration of the arrangement can be adjusted within the distribution region so as to obtain maximum sensitivity in relation to the distribution of the polarization state to be detected on a Poincaré sphere. In this case, the number of detection channels on a Poincaré sphere is a minimum of three channels on the principle of triangulation. Each channel is positioned at a specific detection coordinate on a Poincaré sphere, and the channel output may be considered as proportional to the distance from the detection coordinate. For example, when a right circular polarizer (S3 axis=north pole) is selected as the channel, left circularly polarized light (−S3 axis=south pole) orthogonal on a Poincaré sphere is obtained in the back channel output. The same applies to elliptically polarized light analyzers, where an elliptical analyzer for left ellipses of the southern hemisphere outputs an elliptical analyzer image in which the azimuth angle is orthogonal in real space with the same ellipticity as the northern hemisphere in the back channel.

In the case that the polarization state extends across the entire Poincaré sphere, points may be taken on the orthogonal S1, S2, and S3 axes on the Poincaré sphere, and another optimal solution is a four-channel configuration in which a left circular polarizer of −S3 is added to the linear polarizers on three equidistant axes 0°, 120′, and 240° in real space as described in (A).

Although a method may be used in which the reflected light is split into a plurality of beams and conducted to channels along the optical axis of the reflected light using a partial refection mirror that does not have polarization characteristics, a method may also be used in which the light flux of the reflected light is split into a plurality of beams within its cross section. In such a case, a bundle of polarization retaining fibers may be used, for example, but a total reflecting prism may also be used and designed to function as a phase shifter. Examples of these configurations are described below.

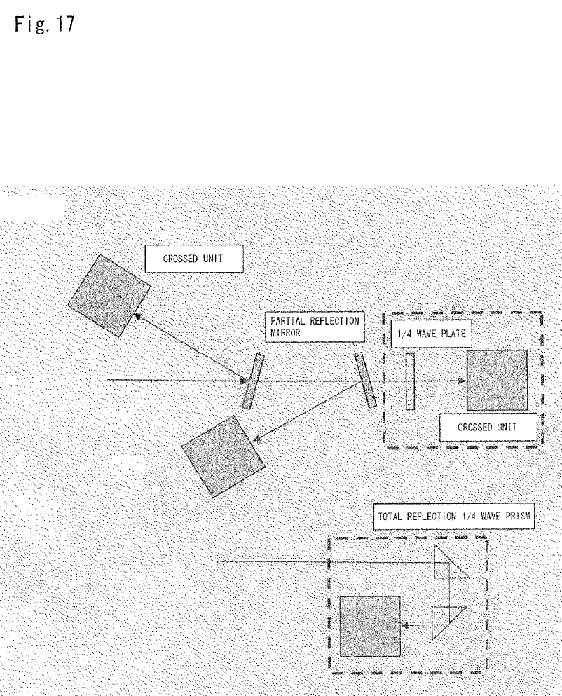

FIG. 17 shows a basic configuration for measuring the Stokes parameters of a two-dimensional image. The portion surrounded by a broken line is an orthogonal unit for detecting circularly polarized light, and comprises a quarter-wave plate and an orthogonal unit. In a configuration in which white light is used as the light source, quarter-wave prisms using total reflection phase jumps with low wavelength dependency may be used instead of a standard quarter-wave plate. A partial reflection mirror inserted in the optical axis is used at an angle of reflection or oblique incidence as close as possible to vertical incidence in order to reduce polarization characteristics. From the light source side, reflectivity is ⅓ and ½, and light is equally distributed in thirds to three orthogonal units to detect the orthogonal polarized components of (horizontal linearly polarized component S₁, vertical linearly polarized component −S₁), (+45° linearly polarized component S_{2, -45}° linearly polarized component −S₂), and (right circularly polarized light S₃, left circularly polarized light −S₃).

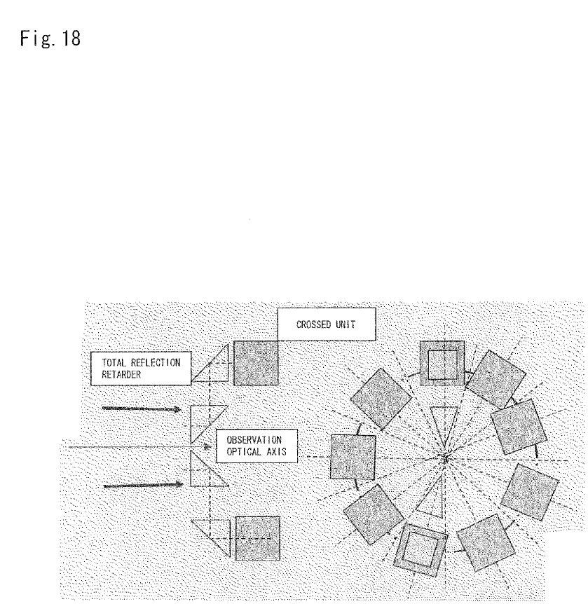

The number of channels is usually increased in order to improve the precision of measurement of the Stokes parameters. In this case, the reflected light may be split along the optical path by a partial reflection mirror, but the prism scheme is suitable for more precision splitting. The nine channels in FIG. 18 give a total of eighteen polarized light image outputs. In actual practice, combining with a partial reflection mirror to split the reflected light along the optical path gives an ample 36 image outputs in the rotating retarder scheme.

The technique of the present invention satisfies the need to assure the precision of polarized light measurements in the range of a group of light beams having essentially the same polarization state as the polarization state of the light beam reflected in the observation orientation among the reflected light flux spreading out from the reflection point. For example, if a polarization camera is far enough away, no special configuration is required, but the spatial resolution of an image is reduced. If a polarization camera is near, on the other hand, the spatial resolution of the image can be made great enough, but the measurement precision of the polarization state is reduced. In order to achieve both advantages, a device provided with a polarization resolution pinhole as shown in FIGS. 10 and 11, or a flat mirror with a pinhole such as that shown in FIG. 13, for example, is advantageous. Similarly, the resolution of measurements of angle of incidence can be increased by further reducing the NA of the detection system. Therefore, the scope of the present invention includes configurations which make use of such arrangements.

The present invention provides a circularly polarized light illumination device in which the surface of an object having a smooth surface (boundary) is uniformly irradiated with right or left circularly polarized light, and all incident light beam components that can be specularly reflected in the observation orientation in accordance with the law of reflection are included for the purpose of measuring the shape and gradient of a sample. The surface of the object target of measurement may include the inner surface of the object. In the configuration of the present invention, the circularly polarized light illumination device has a light source for supplying illumination light to illumination sections that form concave surfaces surrounding an outer surface or convex surfaces facing an inner surface of an object to be measured; and illumination sections for causing light flux that is generated by the light source device and that travels toward an object to be circularly polarized and transmitted.

The circularly polarized light illumination device provided by the present invention is characterized in being used in a shape and gradient measurement method for measuring the shape and gradient of an object by making circularly polarized light incident on a gradient plane constituting a surface of the object, including an inner surface, and using the polarization characteristics of a reflected light beam specularly reflected in a specified observation orientation to form the gradient plane and a three-dimensional gradient angle of the gradient plane. The present circularly polarized light illumination device is characterized in providing a light source device having illumination sections with circular shapes, rectangular shapes, or a combination thereof forming polyhedral shapes comprising a flat surface or a curved surface directly facing the object, for causing a group of circularly polarized light beams made incident on the surface of the object to include all incident light beam components that can be specularly reflected in the observation orientation in accordance with the law of reflection, wherein the sections comprise concave surfaces surrounding an outer surface of the object or convex surfaces facing an inner surface of the object, and essentially perfectly circularly polarized light can be irradiated toward the object through the sections.

The principle of measuring the gradient of a surface of an object using polarized light, which is within the technical scope of the present invention, belongs to a field of precision optical measurement techniques termed “three-dimensional gradient ellipsometry” as newly proposed by the inventors. The technical aspects can be found in the specification of Japanese Patent Application No. 2008-211895 (Patent Document 7), which is the basic application claiming right of priority; and in “Seihansya ni yoru Buttai Hyomen no Keisha Ellipsometry—Seimitsu Jitsu Jikan Keijo Keisoku e no Kihon Gainen” (Gradient Ellipsometry of Object Surfaces by Specular Reflection—Basic Concepts for Precise Real Time Shape Measurement), Kogaku (Japanese Journal of Optics), Vol. 38, No. 4 (2009).

Generally, ellipsometry is known as a method that is capable of making precise measurements of the thickness and refractive index of a thin film on a surface by causing polarized light to be diagonally incident on a flat (thin film) sample to precisely measure the polarization state of “specularly reflected light” that is mirror-reflected in accordance with the law of reflection. In conventional ellipsometric techniques, application is limited to flat samples. In other words, the sample surface is adjusted so that the normal of the surface is within a predetermined incident plane, and at the same time, the gradient of the sample surface within the incident plane achieves an angle of incidence that satisfies the law of reflection in relation to the optical axis of the reflected light beam and the optical axis of the incident light beam of the ellipsometer. The angle of incidence of the light at the time of measurement and the azimuth angle of the incident plane are known variables and are fixed during measurement by adjusting the gradient of the surface of the sample.

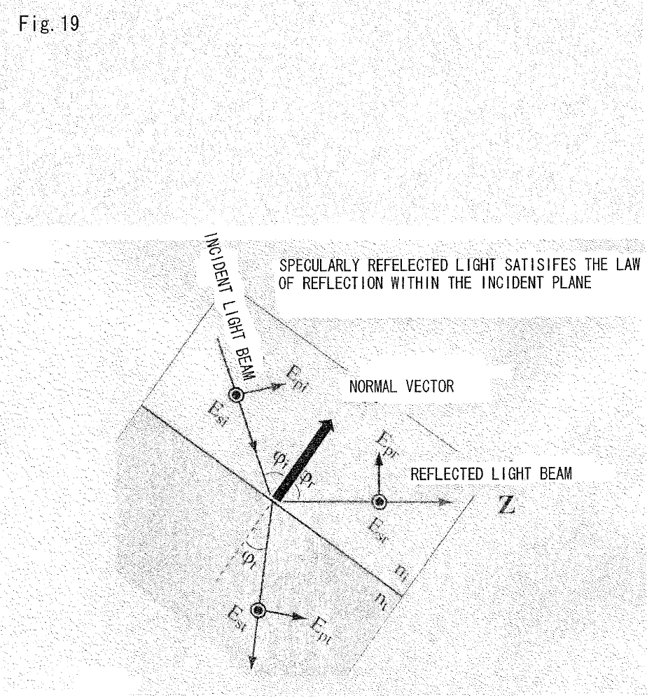

In “three-dimensional gradient ellipsometry,” on the other hand, the concept of ellipsometry conventionally defined as within the incident plane (two-dimensional plane) of a flat sample is expanded to specular reflection from a surface that includes the inner surface of a three-dimensional object. A surface of an object is uniformly irradiated with circularly polarized light, and the specularly reflected light is observed from the z direction. In this case, a “bright” specularly-reflected zero-order light beam component that satisfies the law of reflection is present at an arbitrary reflection point within the surface viewable from the z direction, as shown in FIG. 19. An arbitrary, very small reflection plane that forms a portion of the three-dimensional surface in an arbitrary orientation can be made to match the Incident plane within the surface of the page in FIG. 19 at a rotation of the azimuth angle of −θ to the z-axis as the axis of rotation and using the specularly reflected light beam traveling forward in the observation direction z as a reference. The incident plane is defined as a plane that includes the incident light beam and the normal of the reflection plane, where the incident plane invariably includes the normal vector perpendicular to the reflection plane. It is apparent from FIG. 19 that the angle of reflection (=angle of incidence) is equal to the angle formed by the normal vector and the z-axis, and the normal vector may be determined as long as the azimuth angle θ of the incident plane and the angle of incidence can be determined for arbitrary light beam traveling in the z direction. The shape can be reconstructed by integrating as long as the normal can be determined. Here, the azimuth angle θ and the angle of incidence φ of the incident plane can be determined from the ellipticity angle and the azimuth angle of a polarization ellipse observed by “three-dimensional ellipsometry.”

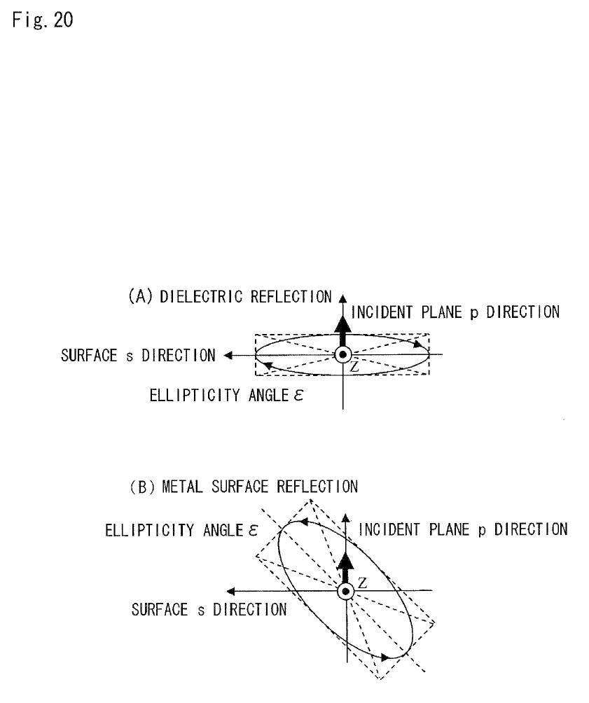

The ellipse of reflected polarized light observed from the z direction under circularly polarized illumination can be illustrated as in FIG. 20. In the case of reflection from a dielectric sample, the major axis of the ellipse tilts 90° from the p direction of the incident plane, and the minor axis of the ellipse therefore constantly matches the p direction of the incident plane (the upper drawing of FIG. 20). Hence, the azimuth angle (the azimuth angle to the z axis as the axis of rotation) of the incident plane including the normal vector can be read directly. In the case of a metal, the major axis of the ellipse constantly tilts about 45° as shown in the lower drawing of FIG. 20. The offset angle of the major axis of the ellipse is a constant determined by the optical characteristics of the reflection plane, and can be obtained by ellipsometric calculation. Therefore, the azimuth angle of the normal vector can be determined for all substances from the azimuth angle of the major axis of the ellipse.

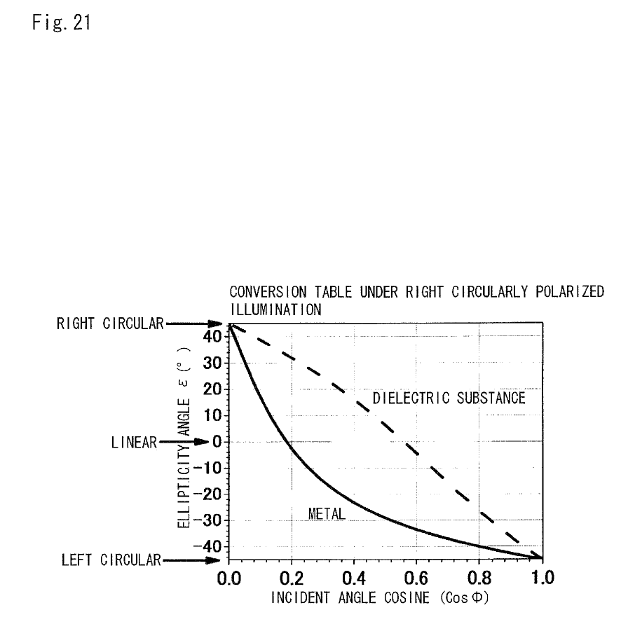

The ellipticity angle of the observed ellipse is a monotonic function of the angle of incidence of the light beam. FIG. 21 shows a conversion table for converting an ellipticity angle observed with incident right circularly polarized light to the cosine of the angle of incidence. The cosine of the angle of incidence matches the z component of the direction cosine of the unit normal vector of the reflection plane. As shown by this calculation example, most substances have a transformation curved between the solid line (metals) and the broken line (dielectric substances). The ellipse shown in FIG. 20 shows a case in which the angle of incidence φ of FIG. 21 is less than 60°, producing a right-handed ellipse for a dielectric substance and a left elliptical ellipse for a metal. Thus, the cosine of the angle of incidence can be directly read from the ellipticity angle.

The sign of the ellipticity angle is determined by the direction of rotation of the ellipse, where positive indicates right polarized light and negative indicates left polarized light. The range of angles of incidence (0° to 90°) in the conversion table matches the range of ellipticity angles (−45° to +45°). Therefore, the normal vector of the reflection plane can be precisely determined with the same angle precision from the ellipticity angle and the azimuth angle of the specularly-reflected elliptically-polarized light.

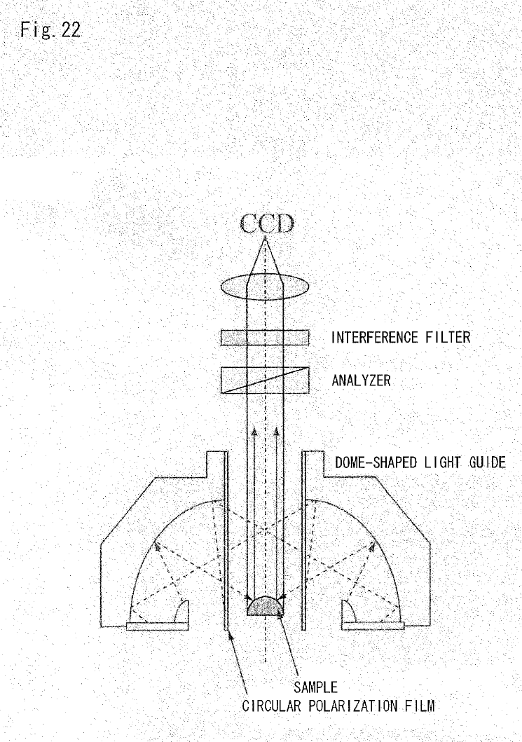

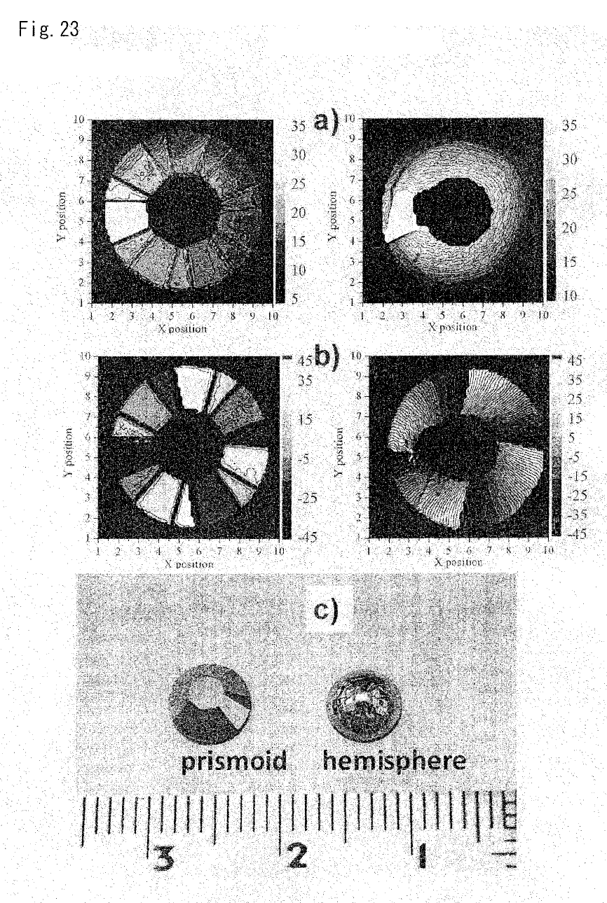

FIG. 22 shows an experimental device used in a measurement experiment carried out on the basis of the disclosure of Japanese Patent Application No. 2008-211895, which is the basic application claiming right of priority. In this device, a circularly polarized illumination device was fabricated by winding a circular polarization film cylindrically and inserting the cylindrical film into a commercially-available dome-shaped illumination device. The polarization ellipse of specularly reflected light is observed using the rotating analyzer method in combination with polarizers (analyzers) made of Polaroid sheets, a 633-nm wavelength interference filter, and a CCD detector. The left and right sides of FIG. 23 show observation results of a prismoid and a hemisphere. From the top of the drawing, a) is the observed ellipticity angles, b) is the observed azimuth angles, and c) is a photograph of the samples.

As shown in grayscale, gradient observation with sufficient resolution is demonstrated by a sample having a diameter of about 6 mm using a simple observation device. Disturbances in the polarization state in the left center portion of the observation results for the samples were caused by nonuniformities in the incident circularly polarized light at the seams of the circularly polarization films. The grayscale of the observed ellipticity of FIG. 23a) shows a range of 5° to 35° in the left diagram and a range of 10° to 35° in the right diagram. The ellipse observed using the right circularly polarized illumination device is left-handed and the ellipticity angle is in the negative region, but the angle is shown as positive because the measurement data were obtained using the rotating polarizer method in which right- and left-handed polarized light cannot be distinguished. In the hemisphere sample on the right, the region near direct incidence and the region of oblique incidence in which the peripheral part of the sample is sheer are not observed because of deficient illuminating light that can be spatially and specularly reflected.

It is apparent from this experiment that a configuration of a circularly polarized light illumination device that is advantageous for three-dimensional gradient ellipsometry requires that perfectly circularly polarized light be included to the extent possible as an incident light beam component that can be reflected as an observable specular reflection component in order to accurately transfer the gradient information of the surface of the object to the reflected polarization ellipse. The measurement sensitivity is essentially determined by the measurement sensitivity of ellipsometric measurements.

The high precision of ellipsometry can be described using Malus' law, which expresses extinction by a set of polarizers because polarized light made incident on the surface of a sample as a probe is perfectly polarized light with a single established polarization state (Non-patent Document 8: “Principal Angle-of-Incidence Ellipsometry”, K. Kinoshita and M. Yamamoto, Surf. Sci. 56, 64-75 (1976)).

Transmission intensity can be expressed as follows when a polarizer and an analyzer are arranged in a straight line, the transmission axis of the polarizers is fixed at an azimuth angle of 0°, and θ is the orientation of the transmission axis of the analyzer.

I(θ)=I₉₀+(I₀−I₉₀)cos² θ| [Formula 33]

Where

[symbol]

[Formula 34]

I₉₀ is the transmission intensity of the unpolarized component at the crossed Nicols angle of θ=90°, and I₀ is the transmission intensity at the parallel Nicols angle of θ=0°.

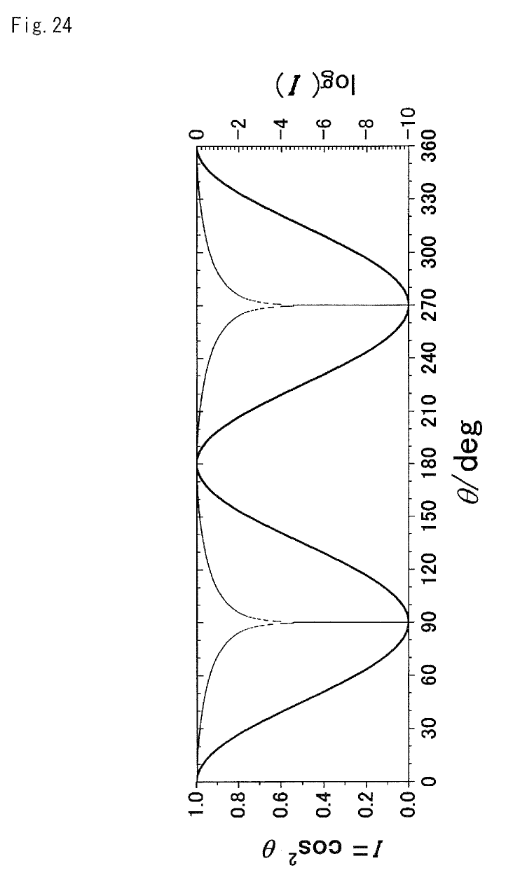

Change in this intensity follows the well-known cosine-squared rule of Malus' law, as shown by the solid line in FIG. 24. The change in intensity extends several orders of magnitude, as shown by the broken line when shown as a logarithm according to the scale on the right vertical axis. The change in intensity is abrupt near the extinction position at an azimuth angle of 90°. and extinguished using a polarization prism with a good extinction coefficient, decays to a level that can be observed with the unaided eye even when the light is laser light.

The ratio of the amplitude reflectances of the p- and s-components at the reflection plane is measured by ellipsometry.

$$\frac{{r}_{p}}{{r}_{s}} ≡ ρ = tan ψexp ( iΔ ) \qquad [ Formula 35 ]$$

An example of an extinction method known for high precision will be described using the arrangement P (polarizer)-S (sample)-C (quarter-wave plate)-A (analyzer). In this PSCA arrangement, C is fixed at an azimuth angle of 45°. It is assumed that the azimuth angles of P and A have been mutually adjusted to achieve perfect extinction. At this point, Ψ can be determined from the azimuth angle of the polarizer, and Δ can be determined from the azimuth angle of the analyzer. In other words, the intensity of the p- and s-polarized components after reflection at the surface of the sample is equal when the azimuth angle of the polarizer is tilted Ψ from the p direction, and the major axis of the reflected elliptically polarized light is an azimuth angle of 45° regardless of the value of Δ. Therefore, the ellipse at an arbitrary ellipticity angle is converted by the quarter-wave plate fixed at an azimuth angle of 45° to linearly polarized light that is tilted at the ellipticity angle from the neutral axis of the quarter-wave plate. Here, the ellipticity angle is equal to Δ/2, and Δ is determined from the azimuth angle of A at which linearly polarized light is extinguished.

As a result, the extinction method makes use of the fact that extinction by linearly polarized light is achieved and minimum intensity obtained under the condition that the azimuth angles of the polarizer and the analyzer has been adjusted correctly according to the two variables to be measured. Therefore, indicating the change in intensity near the extinction position θ=90° by the logarithmic scale in FIG. 24, the sharpness of the fall in

Intensity I [Formula 36]

establishes the determination precision of the extinction azimuth angle. The minimum transmission intensity at this extinction position is determined by the extinction performance of the polarizer.

The performance of the polarizer is expressed by the extinction rate defined as the ratio of the minimum transmission intensity to the maximum transmission intensity.

Ex=I₉₀/I₀ [Formula 37]

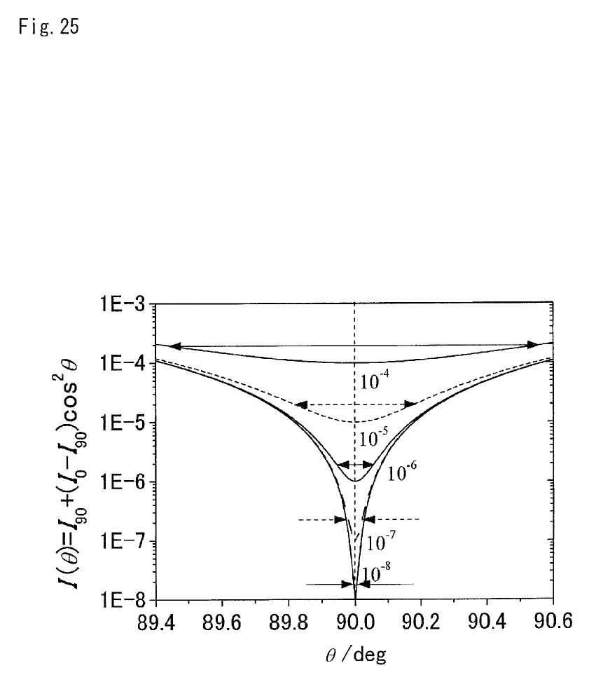

(The extinction rate may also be defined by the reciprocal of the above). FIG. 25 shows the change in the azimuth angle near the extinction position of

Intensity I [Formula 38]

observed by Malus' law using polarizers having various extinction rates.

Intensity I [Formula 39]

varies abruptly symmetrically to the extinction position. The detection sensitivity is determined by the detectable rate of change in the intensity. As shown in FIG. 25, when a change of 10% can be detected from

I₉₀| [Formula 40]

the angle widths indicated by the arrows are produced. Treating the

Extinction rate Ex [Formula 41]

as a function of the polarizer, this width is equal to

0.1\sqrt{Ex}[Formula 42]

The

Extinction rate Ex [Formula 43]

can reach 10⁻⁴ to 10⁻⁵ using a Polaroid sheet polarizer, 10⁻⁵ to 10⁻⁶ using a Glen-Thompson polarizer or other prism-type polarizer, and 10⁻⁷ to 10⁻⁸ using a prism-type polarizer and specially selecting the location and orientation for the prism type.

Light of the minimum transmission intensity

I₉₀| [Formula 45]

determining the

Extinction rate Ex [Formula 44]

in this extinction state comprises an unpolarized component, showing that in addition to the fact that the linearly polarized light generated by the polarizer, is a partially linearly polarized light that includes an unpolarized component, albeit a small amount, even were this an ideally linearly polarized light, the analyzer scatters it and a fixed unpolarized component passes through the analyzer. The sensitivity characteristics during extinction in ellipsometry equally hold true because a phase shifter operates to make even in the case of arbitrary elliptically polarized light in which the shape of the perfectly polarized light includes circularly polarized light is made linearly polarized light by the action of a phase shifter. Specifically, in an ellipsometric system, the measurement error can be expressed as the error in measurement of the

Degree of polarization V [Formula 46]

of the polarized light.

Generally, the

Degree of polarization V [Formula 47]

is calculated using the intensity

Iᵤ [Formula 48]

of the unpolarized component comprising an arbitrary partially polarized light, and the perfectly polarized component

Iₚ [Formula 49]

in the expression

V=Iₚ/(Iₚ+Iᵤ) [Formula 50]

Here, the minimum transmission intensity

I₉₀| [Formula 51]

at the time of extinction is equal to

Iᵤ [Formula 52]

and the maximum transmission intensity

I₀| [Formula 53]

is equal to

Iₚ+Iᵤ| [Formula 54]

Therefore, the extinction rate is

Ex=I₉₀/I₀=Iᵤ/(Iₚ+Iᵤ)=1−V|, [Formula 55]

and the measurement sensitivity is proportional to

0.1\sqrt{1−V} [Formula 56]

The polarization capability of a circular polarizer is determined by the polarization capability of the polarizer to be used. For the following reasons, dependency on the irradiation angle dependency occurs in the polarization state, and the polarization state varies from circularly polarized light except for vertical incidence. This is a factor in degrading the degree of polarization of light that has been formed into an image in the imaging position.

1. The permissible angle range of the polarizer is limited, and is about a maximum of ±15° using a Glen-Thompson prism (see http://www.b-halle.de/EN/Catalog/Polarizers/Glan-Thompson Polarizing Prisms.php)

2. The phase angle of the phase shifter theoretically depends on the angle.

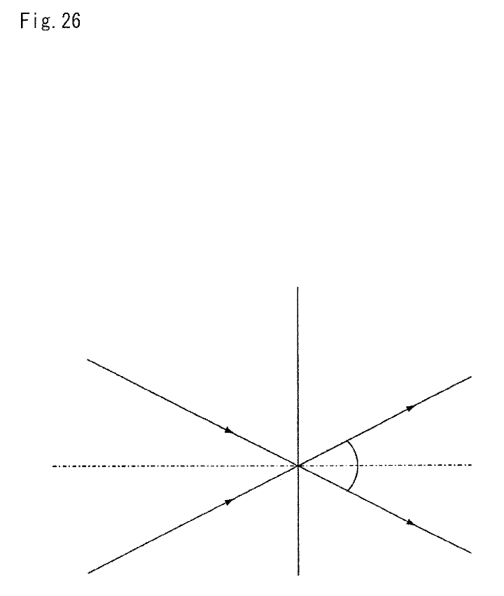

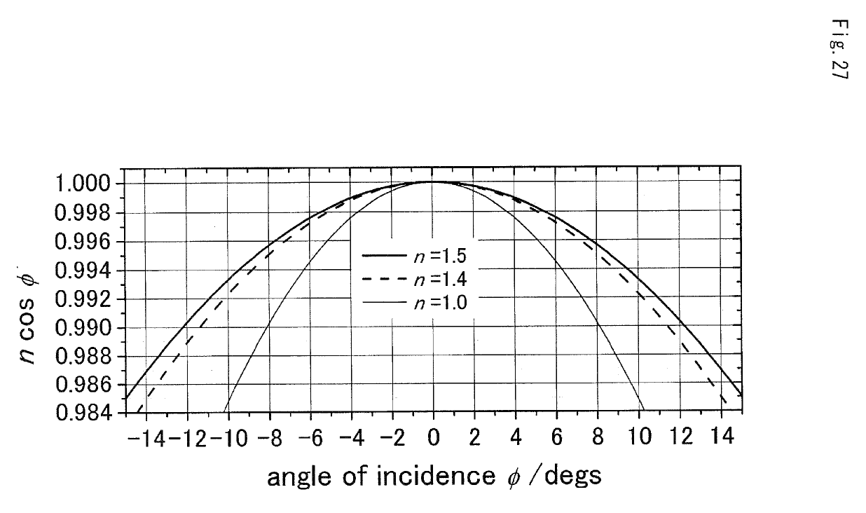

In particular, the phase angle of a phase shifter that uses birefringence theoretically depends on the angle of incidence, and the range of permissible angles is thus limited depending on the required precision, as shown in FIG. 26. FIG. 27 shows that using a light source device having illumination sections configured in accordance with the present invention so as to include a light source, optical elements for guiding light to the sections, and a circular polarizer in the stated order, can provide a function capable of emitting perfectly polarized light having a predetermined degree of polarization from the sections as a light beam flux at the angle of incidence in a predetermined range of angles. FIG. 27 shows calculation examples for average refractive indices of 1.5, 1.4, and 1.0. When a polarizer extinction rate of 10⁻⁶ is set as a reference, a variation in the phase angle of 10⁻³ radians to 10⁻⁴ radians is demanded. That is, the variation in phase angle is from 0.1% to 0.01%. Reading from FIG. 27, these phase angles mean that the contact angles from the optical axis of the phase shifters are in a range ±12° and ±4°, respectively. The data shown in FIG. 27 show that a light source device having illumination sections configured in accordance with the present invention can illuminate the object with a group of circularly polarized light flux with a degree of polarization of essentially 99% or greater.

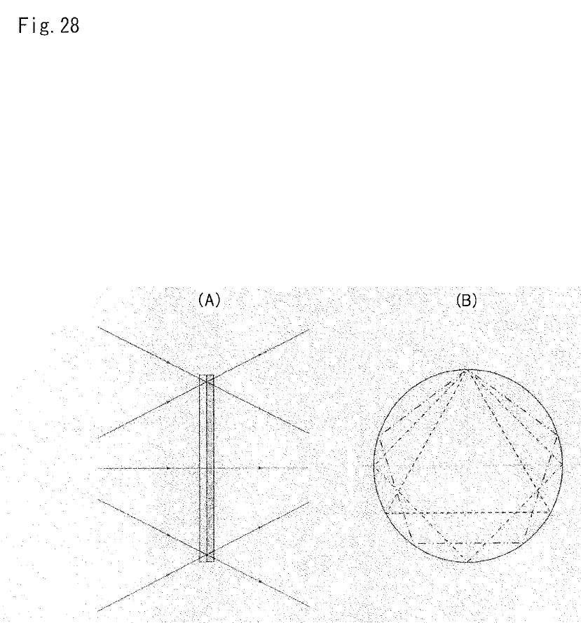

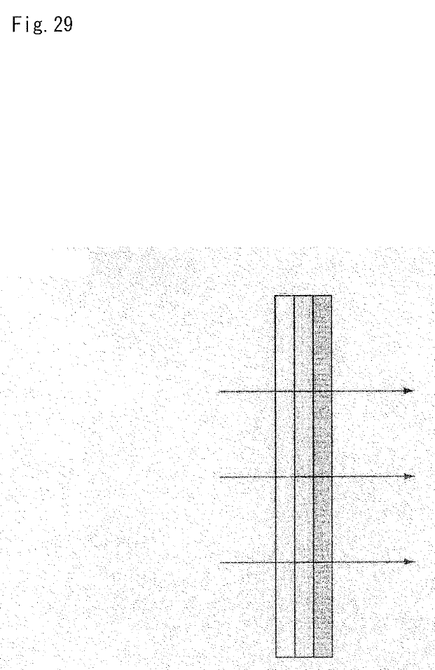

Based on these factors, the angle of incidence or the output angle of the light beams in relation to these polarizers must be kept within a predetermined permissible range of angles in order to generate perfectly circularly polarized light with a predetermined precision. Illumination sections that satisfy these conditions may constitute an illumination region as regular polygonal elements inscribed within a circle indicating the permissible angle, as shown by the example in FIG. 28. Thus, the illumination sections of the light source device may be any regular polygon inscribed within a circle, or a combination thereof forming a polyhedral section. Specifically, the illumination region must be divided in a predetermined range of angles according to the required precision. The illumination sections may be compactly configured in a simple manner by laminating circular polarizers to a surface-emitting light source as shown in FIG. 29. This configuration is particularly useful when measuring a surface comprising an inner surface of a measured object; thus, a light source device having illumination sections in accordance with the present invention may have a configuration that includes at least a light source that is essentially a planar light source composed of an array of point light sources, and/or a surface-emitting light source, and a circular polarizer in the stated order.

As a result of the above study, a circularly polarized light illumination device advantageous for three-dimensional gradient ellipsometry must satisfy the following conditions:

(1) that the device can supply all incident light beams for generating reflected light beams that are specularly reflected at the surface of an object viewable from the observation direction and that travel forward in the observation direction;

(2) that the illumination region comprise a plurality of illumination sections in order to form perfectly circularly polarized light on the surface of the object from the illumination light produced in (1) above;

(3) that the illumination sections have a function for emitting perfectly circularly polarized light of a predetermined precision in a predetermined range of angles from the sections toward the object, and that the polarization state of the propagated light not be disturbed by the optical path of the incident light beams after the circular polarizers of the sections; and

(4) that the polarization state of the elliptically polarized light generated as a result of specular reflection at the object not be disturbed in the reflection optical path.

The following requirements should be added depending on the mode of usage.

In three-dimensional gradient ellipsometry, the optical characteristics of an object are generally already known. However, adding a function for determining the optical characteristics of an object using existing ellipsometric analysis can expand the applicable range by adding a mechanism for obtaining the optical characteristics of an object.

In this case, there is an additional requirement (5) that there be an illumination angle origin reference for giving the azimuth angle and the angle of incidence of an already-known reference light beam.

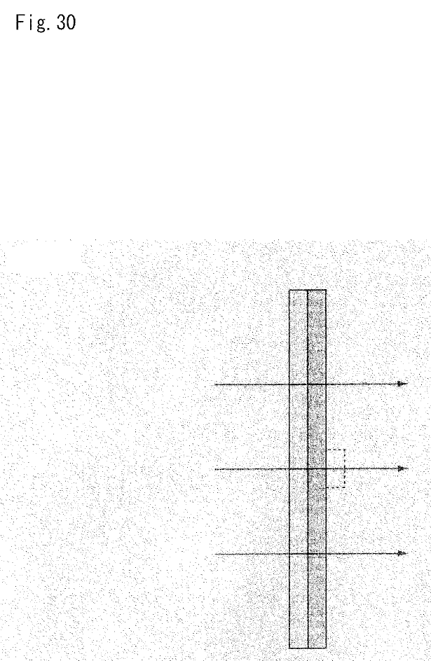

FIG. 30 shows a configuration example for this purpose. FIG. 30 shows that a device may be configured to have an illumination angle origin reference within the illumination sections of the light source device according to the present invention. The broken line portion in the drawing is an illumination angle origin reference, and a function is provided for specifying the coordinates in an image while detecting the polarized light image by varying the characteristics of the transmission wavelength or the transmission intensity from other regions.

A function is provided for temporally or spatially selecting the circular polarization state of illumination light flux using right circularly polarized light and left circularly polarized light in a mode of usage for simplifying the analysis algorithm and improving measurement precision by using the fact that the offset angle of the elliptical orientation of an absorptive body switches symmetrically to the orientation of the incident plane. In view of the above, a useful requirement is that

(6) a mechanism is provided for temporally or spatially selecting the circular polarization state of the illumination light flux using right circularly polarized light and left circularly polarized light.

Thus, according to the present invention, a circularly polarized light illumination device used in shape and gradient measurement methods for measuring the shape and gradient of an object by making circularly polarized light incident on a gradient plane constituting the surface of an object including an inner surface, and using the polarization characteristics of reflected light beams specularly reflected in a specified observation orientation to form the gradient plane and a three-dimensional gradient angle of the gradient plane, may clearly be configured so as to provide illumination sections with circular shapes, rectangular shapes, or a combination thereof forming polyhedral shapes comprising a flat surface or a curved surface directly facing the object, for causing a group of circularly polarized light beams made incident on the surface of the object to include all incident light beam components that can be specularly reflected in the observation orientation in accordance with the law of reflection, wherein the sections comprise concave surfaces surrounding an outer surface of the object or convex surfaces facing an inner surface of the object, and essentially perfectly circularly polarized light can be irradiated toward the object through the sections. It can be understood that the present invention provides a circularly polarized light illumination device characterized in comprising a light source device having such a configuration.

Optimum embodiments for satisfying the above are described below.

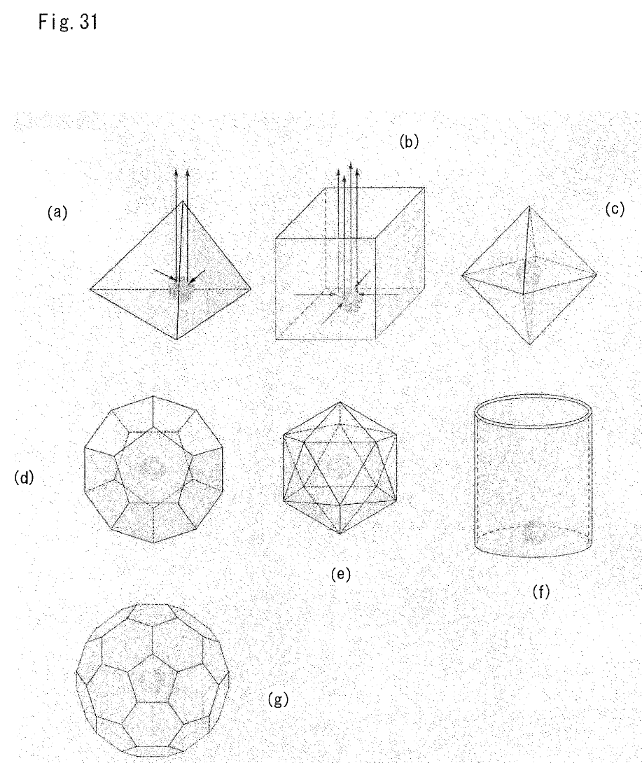

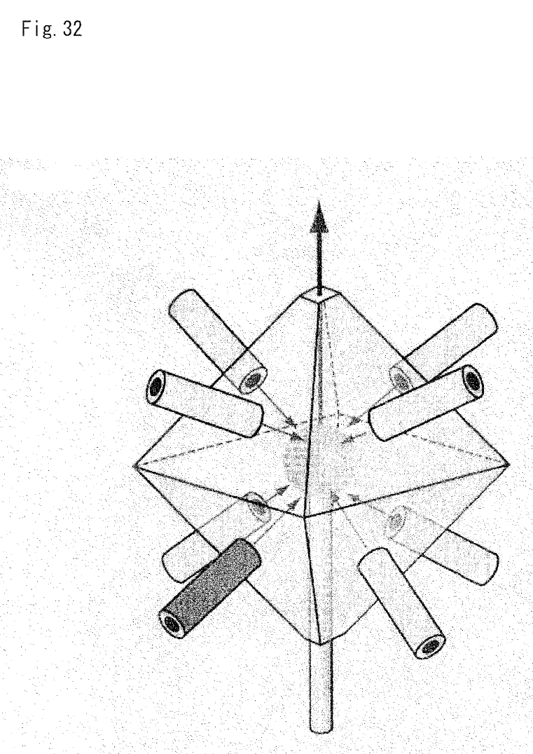

Various configuration examples can be provided of circular shapes, rectangular shapes, or a combination thereof forming polyhedral shapes comprising a flat surface or a curved surface directly facing the object to be measured. FIG. 31 shows configuration examples of illumination regions in which regular polygons form illumination sections. FIG. 31 shows cases of polygons with m number of sides, where m=4, 6, 8, 12, and 20. FIG. 31 shows cylindrical shapes made of circular polarization film, and a soccer-ball shaped polygon having a combination of regular pentahedrons and hexahedrons. In FIG. 31, the sphere in the center portion represents a sample in the case that the outer surface of a sample is being observed, and a light source in the case that the inner surface is being observed. In the case of the regular tetrahedrons and the regular hexahedrons, the arrows show the travel directions of the light beams when the sphere is the sample. When the sphere is a light source, the direction of the light beams toward the sphere is reversed to direct all of the light beams outward through the polyhedron sections. The light source of the present invention may be configured by arranging optical fiber elements at predetermined angles to make light perpendicularly incident on the illumination sections. FIG. 32 shows the case of a regular octahedron as a configuration example in which a fiber light source is combined with a polyhedron configuration. In FIG. 32, the sphere in the center portion represents a sample in the case that the outer surface is being observed.

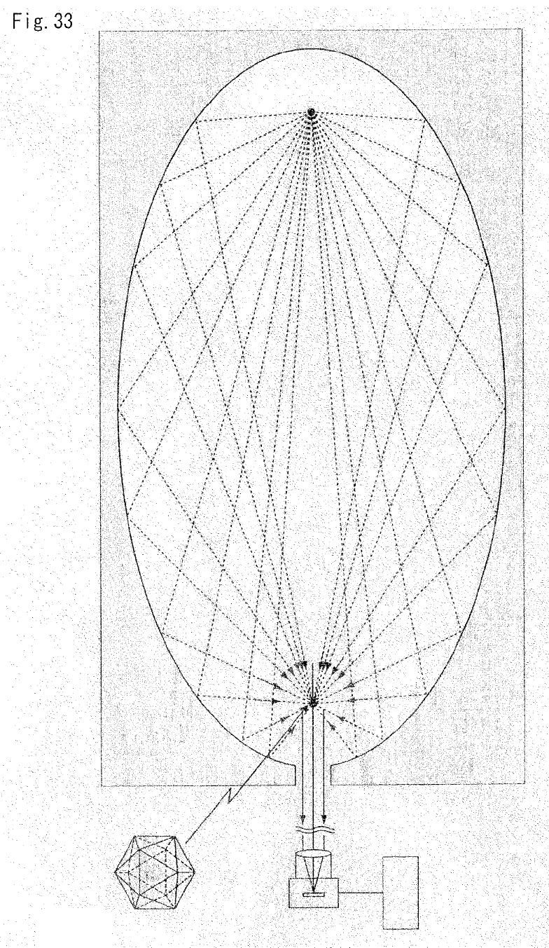

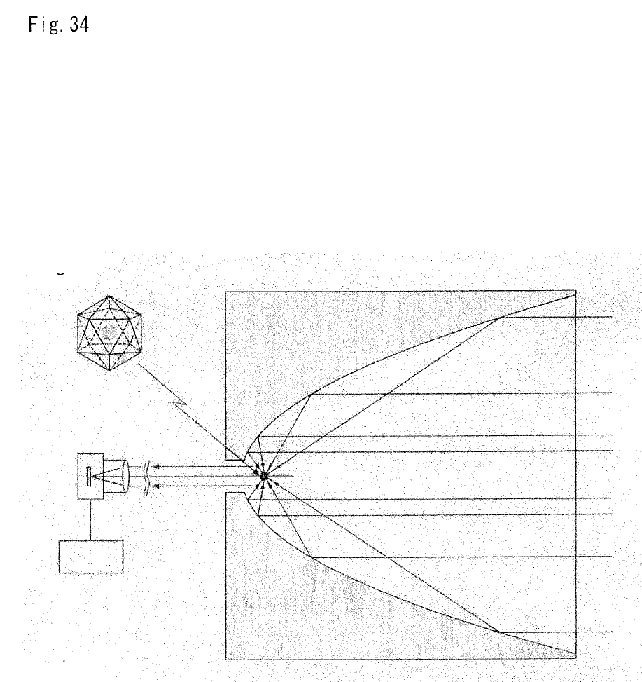

In the circularly polarized light illumination device of the present invention, the light source device may be configured to include a light source mechanism for generating light flux that diverges from at least a single point and a rotating ellipsoidal reflection mirror. The divergence point and the position of the object may be arranged in alignment with the focal point of the rotating ellipsoidal reflection mirror to make light perpendicularly incident on the illumination sections by causing the illumination light beams to converge on the object by reflection. FIG. 33 shows an advantageous example of such a configuration. The specific example of FIG. 33 shows from left to right a polarization camera, a polyhedral illumination section inside which a sample has been placed, and a circularly polarized light illumination device comprising a rotating ellipsoidal mirror and a point light source. In another circularly polarized light illumination device of the present invention, the light source device may be configured to include a light source mechanism for generating at least parallel illumination light flux and a rotating parabolic mirror. The position of the object may be arranged in alignment with the focal point of the rotating parabolic mirror to make light perpendicularly incident on the illumination sections by causing the illumination light beams to converge on the object by reflection. FIG. 34 shows an advantageous example of such a configuration. The specific example of FIG. 34 shows from left to right a polarization camera, a polyhedron illumination section inside which a sample has been placed, and a circularly polarized light illumination device comprising a light source mechanism (not shown) for generating at least parallel illumination light flux and a rotating parabolic mirror.

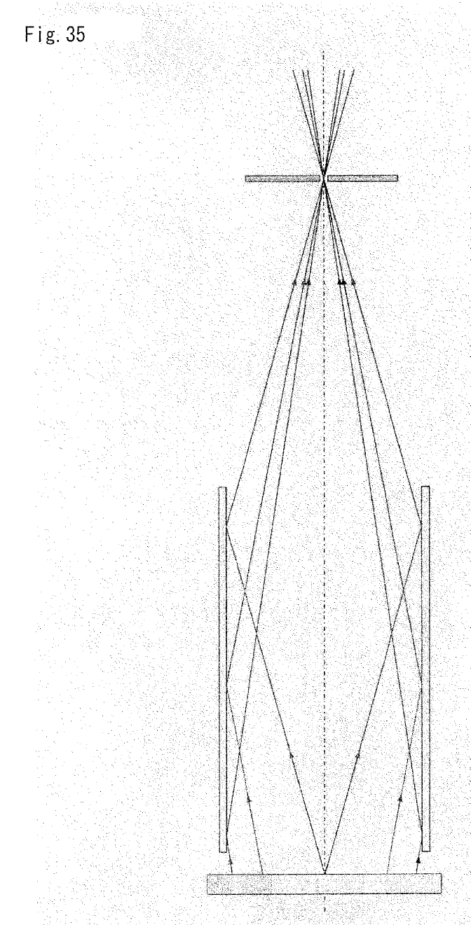

FIG. 35 shows an example for observing the shape of an inner surface. In the present example, a fixed circularly polarized panel light source and a diaphragm can be used to carry out single-process imaging using a polarization camera. The fixed circularly polarized panel light source may include at least a substantially planar light source in which point light sources are arrayed, and/or a surface-emitting light source, and a circular polarizer in the stated order.

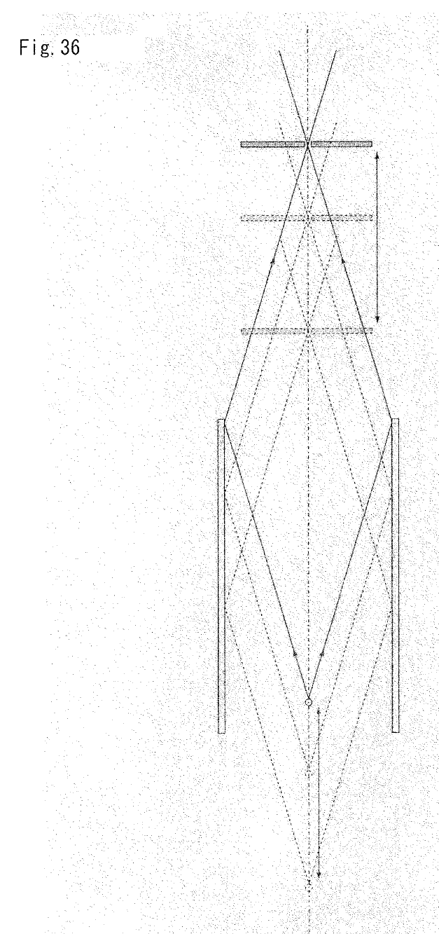

FIG. 36 shows another example for observing the shape of an inner surface. In the present example, a circularly polarized light source and a diaphragm are used to carry out repeated imaging by driving and scanning either the light source or the diaphragm rectilinearly on the axis of rotational symmetry of a sample. The circularly polarized light source may include a light source, optical elements for directing light to the sections, and a circular polarizer in the stated order, and be provided with a function capable of emitting perfectly circularly polarized light having a predetermined degree of polarization from the sections as a light beam flux at the angle of incidence in a predetermined range of angles; be capable of illuminating an object with a group of circularly polarized light beam flux in which the degree of polarization is essentially 99% or higher; have illumination sections of the light source device that form polyhedral sections having any regular polygonal shape or a combination thereof inscribed in a circle; have optical fiber elements arranged at predetermined angles to make light perpendicularly incident on the illumination sections; or include at least a substantially planar light source in which point light sources are arrayed, and/or a surface-emitting light source, and a circular polarizer in the stated order.

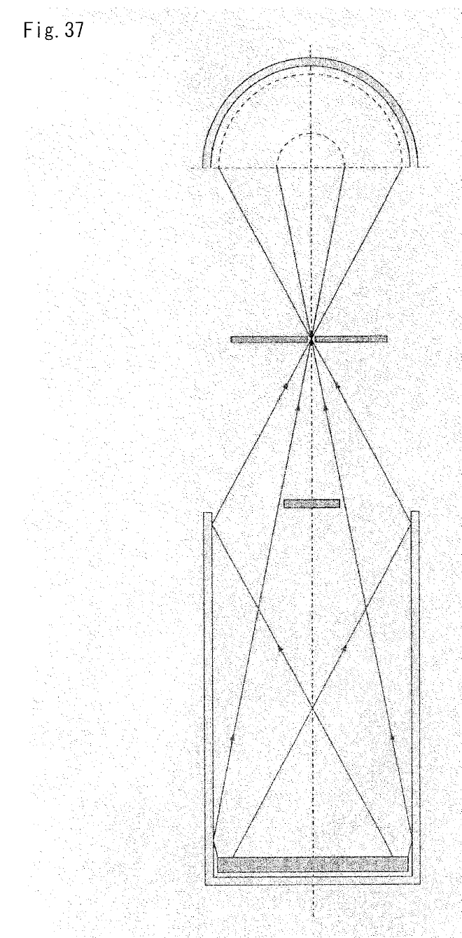

FIG. 37 shows an example for observing the shape of an inner surface in which one end has been sealed off. A circularly polarized light panel, a beam stop, and a diaphragm can be used to carry out single-process imaging using a polarization camera.

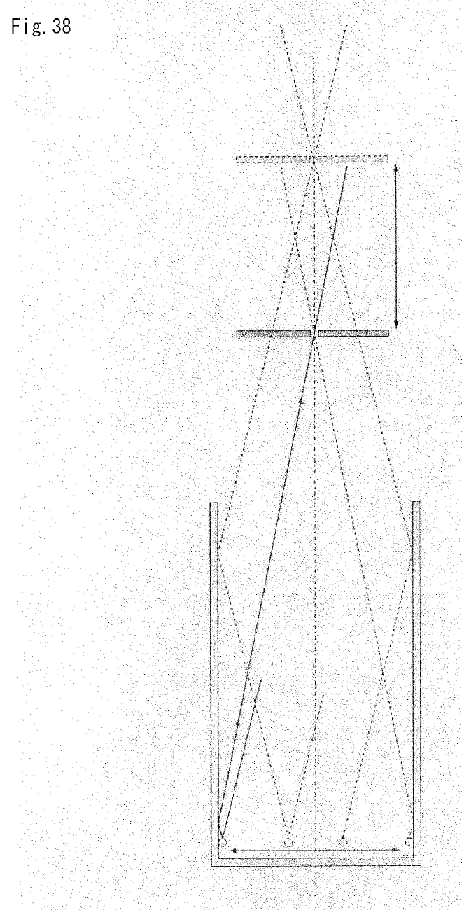

FIG. 38 shows another example for observing the shape of an inner surface in which one end has been sealed off. A circularly polarized light source and a diaphragm are used to carry out repeated imaging by driving and scanning either the light source or the diaphragm rectilinearly in a predetermined manner.

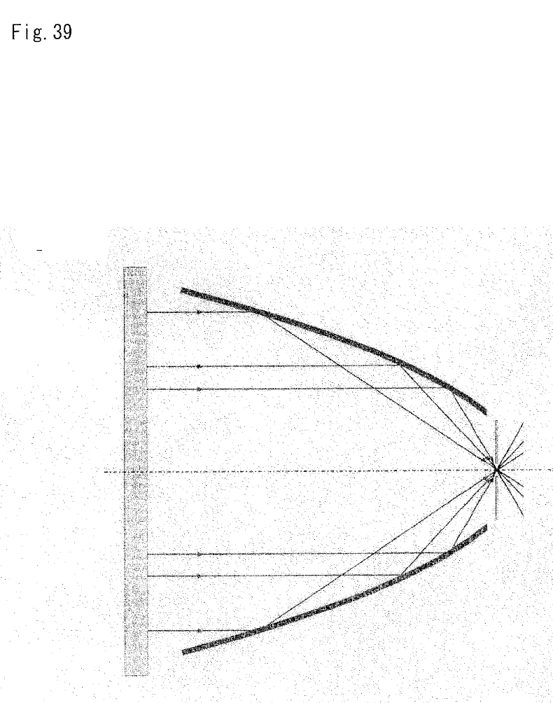

FIG. 39 shows another example for observing the shape of an inner surface for an example in which the shape of the inner surface of a sample forms a paraboloid of revolution. A light source and a diaphragm for irradiating parallel light flux composed of circularly polarized light can be used to carry out single-process imaging using a polarization camera.

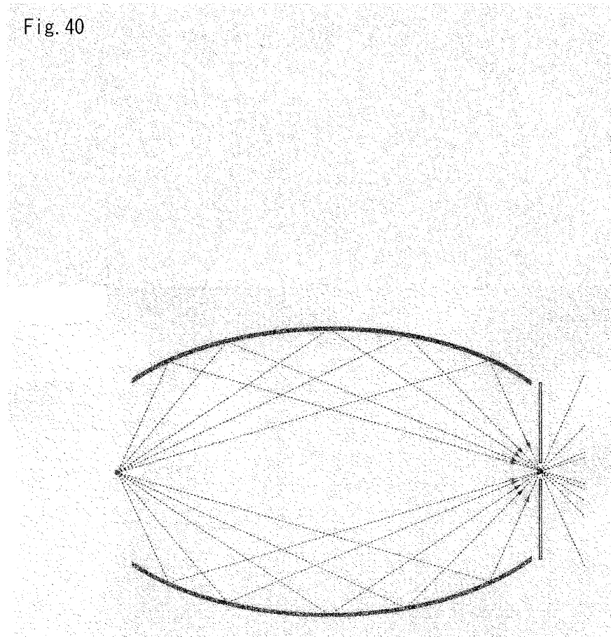

FIG. 40 shows another example for observing the shape of an inner surface for an example in which the shape of the inner surface of a sample forms a paraboloid of revolution. A circularly polarized light source and a diaphragm can be used to carry out single-process imaging using a polarization camera. As described above, the technique of the present invention can be applied to a gradient sensor. As an example of a novel technique according to the present invention, a gradient sensor comprising a reflection plane and arranged on the surface or boundary of an object for which biaxial gradient measurement is desired in real time can be expected to lead to the development of a new area of application. In this case, the sensor may comprise a single-reflection mirror provided with circularly polarized illumination, whereby the biaxial gradient can be read directly. When a rectangular prism having two reflections or a corner cube having three reflections is used, the sensor becomes a single-axis gradient sensor with a round-trip optical path, which can be used to develop remote sensing applications in which a circularly polarized laser is irradiated to measure the gradient using the state of reflected polarized light. Examples of novel applications using telescopes are applications that conventionally required measuring large buildings by triangulation, for which a biaxial gradient sensor comprising a reflection plane and circularly polarized illumination, or a single-axis gradient sensor using a corner cube-type reflection sensor mounted in a required number of locations can be used to measure a plurality of locations simultaneously in real time. This may be expected to be able to measure overall torsion deformation and other dynamic characteristics of a large structure such as a building or a bridge.

### INDUSTRIAL APPLICABILITY

The technique of the present invention can be used in a simple manner with general application for detection of a shape and a gradient and/or measurement and analysis of a shape and a gradient, such as reconstruction of the shape of a sample, by uniformly illuminating a sample with circularly polarized light from the periphery of the sample, measuring the angle of incidence (=angle of reflection) and the gradient of the incident plane at a predetermined reflection point from the state of reflected polarized light observed at the reflection point on the cross-sectional coordinate of a sample by observing the polarization state of reflected light from a spatially fixed direction, and sequentially and smoothly connecting the reflection planes of measured reflection points between measurement points in a cross section of the sample. As a result, it is possible to develop and provide devices for measuring the gradient of the surface of an object, medical diagnostic devices, mammography devices, shape-measuring microscopes, shape-measuring telescopes, monitoring devices for detecting defective shapes and foreign matter in a manufacturing line, and construction of a database of standardized shapes (a database for statistical processing by shape regardless of the size of an object) using integral values (gradients) of the shape of an object.

The circularly polarized light illumination device and circularly polarized light illuminating means of the present invention can be used to develop shape-measuring cameras, shape-measuring telescopes, shape-measuring devices, gradient sensors, and monitoring devices for detecting defective shapes and foreign matter in a manufacturing line.

It is apparent that the present invention can be implemented in applications other than those particularly described in the examples and discussion above. In view of the instructions described above, many alterations and modifications can be made to the present invention, which are therefore included within the scope of the attached claims.

1. An optical device for shape and gradient detection and/or measurement to detect and/or measure a shape and gradient of a surface of an observed object using reflectance optical characteristics of the surface of the object, the optical device for shape and gradient detection and/or measurement characterized in comprising:
an illumination device for causing light surrounding a periphery of the object to be uniformly incident, the light being in a polarized state which includes a substantially already-known perfectly polarized state; and
a polarized light image detection device for detecting a polarized light ellipse of a polarized light component, which includes a perfectly polarized component of a group of light beams specularly reflected by the object surface and emitted at a specific azimuth angle, wherein
a gradient angle with respect to the radiated light beam of the reflection surface is measured by a step 1 in which the orientation of the incident plane is detected from the observed azimuth angle of the polarized light ellipse for the refection surface of the object that forms an incident point for each reflected and radiated light beam, and a step 2 in which the incident angle is detected from the ellipticity value of the polarized light ellipse, which includes the theoretical ellipticity value of the polarized light ellipse.

2. The optical device for shape and gradient detection and/or measurement according to claim 1, characterized in that the illumination device for causing light surrounding the periphery of the object to be uniformly incident, the light being in a polarized state which includes a substantially already-known perfectly polarized state, illuminates circularly polarized light, which includes the perfect circularly polarized light.

3. The optical device for shape and gradient detection and/or measurement according to claim 1, characterized in that step 1, in which the orientation of the incident plane is detected from the observed azimuth angle of the polarized light ellipse, (1) detects the orientation of the incident plane from the observed azimuth angle of the polarized light ellipse, which includes the observed azimuth angle theoretical value of the polarized light ellipse, or (2) causes right circularly polarized light and left circularly polarized light to be incident in a switching fashion in an illumination device for causing light surrounding the periphery of the object to be uniformly incident, the light being in a polarized state which includes a substantially already-known perfectly polarized state, whereby the incident plane orientation is identified by making use of the fact that the observed azimuth angle of the reflected polarized light ellipse, which includes the theoretical value of the observed azimuth angle of the reflection polarized light ellipse, is switched in symmetrical fashion to the incident plane regardless of the reflection optical characteristics of the surface of the object.

4. The optical device for shape and gradient detection and/or measurement according to claim 1, characterized in that the illumination device for causing light surrounding the periphery of the object to be uniformly incident, the light being in a polarized state which includes a substantially already-known perfectly polarized state, includes spatially specified incident light beams as a reference origin of measurement and is capable of specifying the optical characteristics of the reflection surface from the observed value of the polarized light ellipse at a reflection point specified by the polarized light image detection device.

5. The optical device for shape and gradient detection and/or measurement according to claim 1, characterized in that the polarized light image detection device for detecting the polarized light ellipse of a group of light beams reflected by the object surface and emitted at a specific azimuth angle comprises a mechanism capable of extracting an azimuth angle range of the group of light beams having essentially the same polarized light ellipse.

6. The optical device for shape and gradient detection and/or measurement according to claim 1, characterized in that the polarized light image detection device for detecting polarized light ellipses of a group of light beams reflected at the object surface and emitted at a specific azimuth angle has a structure for spatially dividing the reflected light into a plurality of at least three or more groups, assigning a plurality of detectors that can detect specific and mutually different polarized light ellipses, and simultaneously detecting in parallel the polarized light ellipses.

7. The optical device for shape and gradient detection and/or measurement according to claim 1, characterized in comprising a crossed linearly polarized light image detection unit for causing reflected light to be divided by a polarized light beam splitter into a p-component that travels directly forward and a reflected s-polarized light component, causing each of the components to be formed into an image on a two-dimensional detector by an imaging lens, and for drawing out an object image as a crossed polarized light image output.

8. The optical device for shape and gradient detection and/or measurement according to claim 1, characterized in that the polarized light image detection device for detecting polarized light ellipses of a group of light beams reflected at the object surface and emitted at a specific azimuth angle has a mechanism for specifying a light beam position on the object surface by obtaining a reduced projection image of the object.

9. The optical device for shape and gradient detection and/or measurement according to claim 1, characterized in that the polarized light image detection device for detecting polarized light ellipses of a group of light beams reflected at the object surface and emitted at a specific azimuth angle has a mechanism for specifying a light beam position on the object surface by obtaining a magnified projection image of the object.

10. The optical device for shape and gradient detection and/or measurement according to claim 1, characterized in that the polarized light image detection device for detecting polarized light ellipses of a group of light beams reflected at the object surface and emitted at a specific azimuth angle has a mechanism for specifying a light beam position on the object surface by providing a collimator.

11. The optical device for shape and gradient detection and/or measurement according to claim 1, characterized in that the polarized light image detection device for detecting polarized light ellipses of a group of light beams reflected at the object surface and emitted at a specific azimuth angle has a mechanism for specifying a light beam position on the object surface by arranging the device essentially at infinite distance.

12. The optical device for shape and gradient detection and/or measurement according to claim 1, characterized in that the polarized light image detection device for detecting polarized light ellipses of a group of light beams reflected at the object surface and emitted at a specific azimuth angle has a mechanism for specifying a light beam position on the object surface by providing a pinhole.

13. The optical device for shape and gradient detection and/or measurement according to claim 1, characterized in being a medical diagnostic device including mammography for detecting and identifying a specific change in a surface gradient angle caused by a variety of pathological abnormalities including malignant tumors, an object of detection and/or measurement being a human body or a portion of a human body including a breast.

14. The optical device for shape and gradient detection and/or measurement according to claim 1, characterized in that dynamic characteristics are extracted by imparting deformation caused by a predetermined stress by a dynamic process including a change in orientation of the observed object, which includes a patient, and detecting and/or measuring a change in the gradient angle before and after deformation.

15. The optical device for shape and gradient detection and/or measurement according to claim 1, characterized in that a change in the optical characteristics of a reflection surface is detected and/or measured using the illumination light as white light and the surface of an observed object, including skin, as a substantially reflective surface, taking into account that the depth of penetration from such a surface changes with the wavelength.

16. A method for optical shape and gradient detection and/or measurement to detect and/or measure a shape and a gradient of a surface of an observed object using reflectance optical characteristics of the surface of the object, the method for optical shape and gradient detection and/or measurement characterized in comprising:
using an illumination device to cause light surrounding a periphery of the object to be uniformly incident, the light being in a polarized state which includes a substantially already-known perfectly polarized state;
using a polarized light image detection device to detect a polarized light ellipse of a polarized light component, which includes a perfectly polarized component of a group of light beams specularly reflected by the object surface and emitted at a specific azimuth angle;
measuring a gradient angle with respect to the radiated light beam of the reflection surface by detecting the orientation of the incident plane from the observed azimuth angle of the polarized light ellipse for the refection surface of the object that forms an incident point for each of the reflected and radiated light beams, and detecting the incident angle from the ellipticity value of the polarized light ellipse, which includes the theoretical ellipticity value of the polarized light ellipse; and
extracting object information using the fact that the measured gradient angle smoothly varies on the object surface.

17. The method for optical shape and gradient detection and/or measurement according to claim 16, characterized in that a specific change in the surface gradient angle caused by a variety of pathological abnormalities, including malignant tumors, is detected and identified, the object of detection and/or measurement being a human body or a portion of a human body including a breast.

18. The method for optical shape and gradient detection and/or measurement according to claim 16, characterized in that a predetermined deformation is imparted by a process that includes changing an orientation of the observed body, which includes a patient, and detecting and/or measuring a change in the gradient angle before and after deformation.

19. The method for optical shape and gradient detection and/or measurement according to claim 16, characterized in that a change in the optical characteristics of a reflection surface is detected and/or measured using the illumination light as white light and the surface of an observed object, including skin, as a substantially reflective surface, taking into account that depth of penetration from such a surface changes with the wavelength.

20. A method for detecting and/or measuring a shape and gradient, characterized in comprising an optical device for detecting and/or measuring a shape and gradient, used to detect and/or measure a shape and gradient of a surface of an observed object using reflectance optical characteristics of the surface of the object, having:
an illumination device for causing light surrounding a periphery of the object to be uniformly incident, the light being in a polarized state which includes a substantially already-known perfectly polarized state; and
a polarized light image detection device for detecting a polarized light ellipse of a polarized light component, which includes a perfectly polarized component of a group of light beams specularly reflected by the object surface and emitted at a specific azimuth angle;
measuring the gradient angle in relation to light beams radiated from the reflection surface by detecting: the azimuth angle of the incident plane, i.e., the azimuth angle of the normal of the tangent plane, from the azimuth angle of the polarized light ellipse for the reflection surface, i.e., the vicinal face, of the object that forms an incident point for each of the reflected and radiated light beams; and the reflection angle, i.e., the incident angle from the ellipticity value of the polarized light ellipse; and
carrying out an integration operation for smoothly connecting the vicinal faces that form the tangent plane.

21. The method for detecting and/or measuring a shape and gradient according to claim 20, characterized in comprising directly measuring a reflection angle formed with an axis that is an observation direction, and a polarization angle of a projection component on the plane perpendicular to the axis that is the observation direction, for the normal of the tangent plane at the reflection point of the observed object surface, using incident angle dependency of a variation in the polarized light ellipse formed with a single reflection.

22. The method for detecting and/or measuring a shape and gradient according to claim 20, characterized in comprising establishing a partial derivative coefficient at the coordinates of the axis component that is the observation direction as the gradient of the tangent plane at the reflection point on the surface of the observed object.

23. The method for detecting and/or measuring a shape and gradient according to claim 20, characterized in comprising measuring a slope of the normal of the tangent plane at the reflection point on the surface of the observed object; calculating the partial derivative coefficient of the shape and gradient at the reflection point on the object, measuring temporal changes and/or spatial changes in the partial derivative coefficient; and extracting characteristics of the shape and/or characteristics of the gradient by directly using measured values that have been obtained.

24. The method for optical shape and gradient detection and/or measurement according to claim 20, characterized in comprising measuring the gradient of the tangent plane and the shape of the observed object by ellipsometry using a complex amplitude reflectivity ratio calculated using an optical model that expresses optical properties of the observed sample, and the values Ψ, Δ obtained from the ellipticity angle of the reflected polarized light ellipse and from the azimuth angle of the major axis.

25. A circularly polarized light illumination device used in a shape and gradient measurement method for measuring a shape and gradient of an object, the circularly polarized light illumination device characterized in that:
the shape and gradient of the object are measured by making circularly polarized light incident on a gradient plane constituting the object surface, including the inner surface, and using the polarized light characteristics of reflected light beams specularly reflected in a specified observation direction, to form the gradient plane and a three-dimensional gradient angle of the gradient plane, wherein
the circularly polarized light illumination device comprises a light source device; and
the light source device is a light source device having illumination sections with circular shapes, rectangular shapes, or a combination thereof in polyhedral shapes that include a flat surface or a curved surface directly facing the object, wherein
the sections include concave surfaces surrounding an outer surface of the object or convex surfaces facing an inner surface of an object;
circularly polarized light including essentially perfect circularly polarized light can be irradiated toward the object via the sections; and
a group of circularly polarized light beams made incident on the object surface is made to include all incident light beam components that can be specularly reflected in the observation direction in accordance with the law of reflection.

26. The circularly polarized light illumination device according to claim 25, characterized in that the light source device having the illumination sections includes, in the stated order, a light source, optical elements for directing light to the sections, and circular polarizers; and is provided with a function enabling emitting of circularly polarized light, including perfect circularly polarized light having a predetermined degree of polarization, from the sections as incident angle light beam flux in a predetermined angle range.

27. The circularly polarized light illumination device according to claim 25, characterized in that the light source device having the illumination sections is capable of illuminating the object with circularly polarized light beam flux in which the degree of polarization is essentially 99% or higher.

28. The circularly polarized light illumination device according to claim 25, characterized in that illumination sections of the light source device form polyhedral sections having any regular polygonal shape or a combination thereof inscribed in a circle.

29. The circularly polarized light illumination device according to claim 25, characterized in that the light source device has optical fiber elements arranged at predetermined angles and causes light to be perpendicularly incident on the illumination sections.

30. The circularly polarized light illumination device according to claim 25, characterized in that the light source device having the illumination sections includes at least a substantially planar light source in which point light sources are arrayed, and/or a surface-emitting light source, and circular polarizers in the stated order.

31. The circularly polarized light illumination device according to claim 25, characterized in that the light source device includes a light source mechanism for generating light flux that diverges from at least a single point and a rotating ellipsoidal reflection mirror; the divergence point and the position of the object are arranged in alignment with the focal point of the rotating ellipsoidal reflection mirror; and light is made to be perpendicularly incident on the illumination sections by causing the illumination light beams to converge on the object by reflection.

32. The circularly polarized light illumination device according to claim 25, characterized in that the light source device includes a light source mechanism for generating at least parallel illumination light flux and a rotating parabolic mirror; the position of the object is arranged in alignment with the focal point of the rotating parabolic mirror; and light is made to be perpendicularly incident on the illumination sections by causing the illumination light beams to converge on the object by reflection.

33. The circularly polarized light illumination device according to claim 25, characterized in comprising an illumination angle origin reference within the illumination sections of the light source device.

34. The circularly polarized light illumination device according to claim 25, characterized in comprising a function for temporally or spatially selecting a circularly polarized light state of the illumination light flux using right circularly polarized light or left circularly polarized light.

35. A circularly polarized light illumination method used in shape and gradient measurement methods for measuring the shape and gradient of an object in which circularly polarized light is made to be incident on a gradient plane constituting the object surface, including the inner surface, and the polarized light characteristics of reflected light beams specularly reflected in a specified observation direction are used to form the gradient plane and a three-dimensional gradient angle of the gradient plane, the circularly polarized light illumination method characterized in comprising:
using a light source device having illumination sections with circular shapes, rectangular shapes, or a combination thereof in polyhedral shapes that include a flat surface or a curved surface directly facing the object, wherein the sections include concave surfaces surrounding the outer surface of the object or convex surfaces facing the inner surface of an object;
irradiating circularly polarized light including essentially perfect circularly polarized light toward the object via the sections; and
causing a group of circularly polarized light beams made incident on the object surface to include all incident light beam components that can be specularly reflected in the observation direction in accordance with the law of reflection.
